SEQUENCE LISTING (44 sequences)

SEQ ID NO: 1 - protein, 687 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTM

SEQ ID NO: 2 - protein, 687 aa, synthetic construct
  REGION 1..687: note = h(es)E2ectm (human) with substitutions compared to wild-typeM198V, Q398R, F425L, H473R, and A622T
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQPCPINCTHSCVDLDDKGCPAEQRASPLTSIISAVVGILLVVVLGVVFGILIKRRQQKIRKYTM

SEQ ID NO: 3 - protein, 687 aa, Pan paniscus
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQPCPINCTHSCVDLDDKGCPAEQRASPLTSIISAVVGILLVVVLGVVFGILIKRRQQKIRKYTM

SEQ ID NO: 4 - protein, 664 aa, Saimiri boliviensis
QVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLDNTTPVTGASPGGLRELQLRSLTEILKGGVWIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFVGCKKIFGSLAFLPESFDGDPASNTVPLQPEQLHVFETLEEITGYLYISAWPDSLPDLSVFQNLQVIRGRILHNGAYSLTLQGLGISWLGLRSLRELGSGLALIHHNARLCFVHTVPWDNLFRNPHQALLHTANRPEHECVGKDLACHPLCARGHCWGPGPTQCVNCSQFLRGQECVEECRVLQGLPREYVNARHCLPCHPECQPQNGSVTCSGPEADQCVACAHYKDSPFCVARCPSGVKPDLSYMPIWKFPDEEGTCQPCPINCTHSCVDLDDKGCPAEQRASPLTSIISAVVGILLVMVLGLLLGILIKRRQQKIRKYTM

SEQ ID NO: 5 - protein, 687 aa, Pan troglodytes
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEKGTCQPCPINCTHSCVDLDDKGCPAEQRASPLTSIISAVVGILLVVVLGVVFGILIKRRQQKIRKYTM

SEQ ID NO: 6 - protein, 687 aa, Rhinopithecus roxellana
MELAAWCRWGLLLALLPPGAAGTQVCTGTDMKLRLPASPETHLDMLRHLYQGCQVVQGNLELTYLPTDASLSFLQDIQEVQGYVLIAHNQVRQVPLQRLRIVRGTQLFEDNYALAVLDNGDPLNNITPVTGASPGGLRELQLRSLTEILKGGVLIQRNPQLCYQDTILWKDIFHKNNQLAFTLIDTNRSRACHPCSPVCKGSHCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKSCARVCYGLGMEHLREVRAVTSANIQEFAGCKKIFGSLAFLPESFDGDLASNTAPLQPEQLRVFETLEEITGYLYISAWPDSLPDLSILQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQPCPINCTHSCVDLDDKGCPAEQRASPLTSIISAVVGILLVVVLGVVFGILIKRRQQKIRKYTM

SEQ ID NO: 7 - protein, 687 aa, Gorilla gorilla
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPELCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSIFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTM

SEQ ID NO: 8 - protein, 687 aa, Chlorocebus sabaeus
MELAAWCSWGLLLALLPPGAAGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPACKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQPCPINCTHSCVDLDDKGCPAEQRASPLTSIISAVVGILLVVVLGVVFGILIKRRQQKIRKYTM

SEQ ID NO: 9 - protein, 687 aa, Papio anubis
MELAAWCRWGLLLALLPPGAAGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQPCPINCTHSCVDLDDKGCPAEQRASPLTSIISAVVGILLVVVLGVVFGILIKRRQQKIRKYTM

SEQ ID NO: 10 - protein, 687 aa, Macaca mulatta
MELAAWYRWGLLLALLPPGAAGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDLLNNTTPVTGASPGGLRELQLRSLTEILKGGVLIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQSCPINCTHSCVDLDDKGCPAEQRASPLTSIISAVVGILLVVVLGVVFGILIKRRQQKIRKYTM

SEQ ID NO: 11 - protein, 687 aa, Macaca mulatta
  VARIANT 517: note = Xaa can be any naturally occurring amino acid
  VARIANT 647: note = Xaa can be any naturally occurring amino acid
MELAAWYRWGLLLALLPPGAAGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCAXGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQPCPINCTHSCVDLDDKGCPAEQXASPLTSIISAVVGILLVVVLGVVFGILIKRRQQKIRKYTM

SEQ ID NO: 12 - protein, 658 aa, Pongo abelii
  VARIANT 162: note = Xaa can be any naturally occurring amino acid
  VARIANT 215: note = Xaa can be any naturally occurring amino acid
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIERNPQLCYQDTILWKDIFHKNNQLAVTLIDTNRLSGXHPCFFRCVRAPRCWGESSEDCQSLTRTVCAGGCARCKGPLPTDCCHEQCLPSXHGPQAPSALPCLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVFQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARYCLPCHPECQPQNGSVTCFGPEADQCVACAHYKDPPFCVARCPSGVKPDLSYMPIWKFPDEEGTCQPCPINCTHSCVDLDDKGCPAEQRASPLTSIISAVVGILLVVVLGVVFGILIKRRQQKIRKYTM

SEQ ID NO: 13 - protein, 663 aa, Nomascus leucogenys
VCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTLVTGASPGGLRELQLRSLTEILKGGVLIQRNPQLCYQDTILWKDIFHKNNQLALTLIDTNRSRACQPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLSDLSVFQNLQVIRGRILHNGAYSLTLQGLGISWLGLRSLRELGSGLALIHHNNRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVSCAHYKDPPFCVARCPSGVKPDLSYMPIWKFPDEEGTCQPCPINCTHSCVDLDDKGCPAEQRASPLTSIISAVVGILLVVVLGAVFGILIKRRQQKIRKYTM

SEQ ID NO: 14 - protein, 687 aa, Callithrix jacchus
MELAAWCRWGLLFALLPPGAAGTQVCTGTDMKLRLPASPETHLDMLRHLYQGCQVVQGNLELTYLPANASLSFLQDIQEVQGYVLIAHNQVRQVPLQRLRIVRGTQLFEDNYALAVLDNGDPLDNTTPVTGASPGGLRELQLRSLTEILKGGVWIQRNPQLCYQDMVLWKDIFHKNNQLALTLIDTNRSRACHPCSPACKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTVPLQPEQLQVFETLEEITGYLYISAWPDSLPDLSVFQNLQVIRGRILHNGAYLLTLQGLGISWLGLRSLRELGSGLALIHHNARLCFVHTVPWDNLFRNPHQALLHTANRPEHECVGKDLACHPLCARGHCWGPGPTQCVNCSQFLRGQECVEECRVLQGLPREYVNARHCLPCHPECQPQNGSVTCSGPEADQCVACAHYKDSPFCVARCPSGVKPDLSYMPIWKFPDEEGTCQPCPINCTHSCVDLDDKGCPAEQRASPLTSIISAVVGILLFMVLGLLLGILMKRRQQKIRKYTM

SEQ ID NO: 15 - protein, 687 aa, Macaca fascicularis
MELAAWYRWGLLLALLPPGATGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQSCPINCTHSCVDLDDKGCPAEQRASPLTSIISAVVGILLVVVLGVVFGILIKRRQQKIRKYTM

SEQ ID NO: 16 - protein, 671 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSPLTSIISAVVGILLVVVLGVVFGILIKRRQQKIRKYTM

SEQ ID NO: 17 - protein, 671 aa, synthetic construct
  REGION 1..671: note = Human (es)E2ectm-delta 16; with substitutions M198V, Q398R,F425L, H473R, and A622T
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQPCPINCTHSPLTSIISAVVGILLVVVLGVVFGILIKRRQQKIRKYTM

SEQ ID NO: 18 - protein, 687 aa, Felis catus
MELAAWCRWGLLLALLPSGATGTQVCTGTDMKLRLPASPETHLDMLRHLYQGCQVVQGNLELTYLHANASLSFLQDIQEVQGYVLIAHNQVKQVPLQRLRIVRGTQLFEDNYALAVLDNGDPLDSGTPATGAALGGLRELQLRSLTEILKGGVLIQRNPQLCHQDTILWKDIFHKNNQLALMLIDTNRSRACQPCSPACKDSHCWGASSGDCQSLTRTVCAGGCARCKGPQPTDCCHEQCAAGCTGPKHSDCLACLHFNHSGICELHCPALVTYNTDTFESMPNPEGRYTFGASCVTACPYNYLSTDVGSCTLVCPLNNQEVTAEDGTQRCEKCSKPCARVCYGLGMEHLREARAVTSANIQEFVGCKKIFGSLAFLPESFEGDPASNTAPLQPEQLRVFEALEEITGYLYISAWPDSLPNLSVFQNLRVIRGRVLHDGAYSLTLQGLGISWLGLRSLRELGSGLALIHRNSRLCFVHTVPWDQLFRNPHQALLHSANRPEDECAGEGLACYPLCAHGHCWGPGPTQCVNCSQFLRGQECVEECRVLQGLPREYVKDRFCLPCHPECQPQNGSVTCLGSEADQCVACAHYKDPPFCVARCPSGVKPDLSFMPIWKFADEEGTCQPCPINCTHSCADLDEKGCPAEQRASPVTSIIAAVVGILLVVVVGLVLGILIKRRRQKIRKYTM

SEQ ID NO: 19 - protein, 687 aa, synthetic construct
  REGION 1..687: note = Feline (es)E2ectm with substitutions A198V, R398Q, F425L, R473H,and T622A
MELAAWCRWGLLLALLPSGATGTQVCTGTDMKLRLPASPETHLDMLRHLYQGCQVVQGNLELTYLHANASLSFLQDIQEVQGYVLIAHNQVKQVPLQRLRIVRGTQLFEDNYALAVLDNGDPLDSGTPATGAALGGLRELQLRSLTEILKGGVLIQRNPQLCHQDTILWKDIFHKNNQLALMLIDTNRSRACQPCSPVCKDSHCWGASSGDCQSLTRTVCAGGCARCKGPQPTDCCHEQCAAGCTGPKHSDCLACLHFNHSGICELHCPALVTYNTDTFESMPNPEGRYTFGASCVTACPYNYLSTDVGSCTLVCPLNNQEVTAEDGTQRCEKCSKPCARVCYGLGMEHLREARAVTSANIQEFVGCKKIFGSLAFLPESFEGDPASNTAPLQPEQLQVFEALEEITGYLYISAWPDSLPNLSVLQNLRVIRGRVLHDGAYSLTLQGLGISWLGLRSLRELGSGLALIHRNSHLCFVHTVPWDQLFRNPHQALLHSANRPEDECAGEGLACYPLCAHGHCWGPGPTQCVNCSQFLRGQECVEECRVLQGLPREYVKDRFCLPCHPECQPQNGSVTCLGSEADQCVACAHYKDPPFCVARCPSGVKPDLSFMPIWKFADEEGACQPCPINCTHSCADLDEKGCPAEQRASPVTSIIAAVVGILLVVVVGLVLGILIKRRRQKIRKYTM

SEQ ID NO: 20 - protein, 671 aa, Felis catus
MELAAWCRWGLLLALLPSGATGTQVCTGTDMKLRLPASPETHLDMLRHLYQGCQVVQGNLELTYLHANASLSFLQDIQEVQGYVLIAHNQVKQVPLQRLRIVRGTQLFEDNYALAVLDNGDPLDSGTPATGAALGGLRELQLRSLTEILKGGVLIQRNPQLCHQDTILWKDIFHKNNQLALMLIDTNRSRACQPCSPVCKDSHCWGASSGDCQSLTRTVCAGGCARCKGPQPTDCCHEQCAAGCTGPKHSDCLACLHFNHSGICELHCPALVTYNTDTFESMPNPEGRYTFGASCVTACPYNYLSTDVGSCTLVCPLNNQEVTAEDGTQRCEKCSKPCARVCYGLGMEHLREARAVTSANIQEFVGCKKIFGSLAFLPESFEGDPASNTAPLQPEQLQVFEALEEITGYLYISAWPDSLPNLSVLQNLRVIRGRVLHDGAYSLTLQGLGISWLGLRSLRELGSGLALIHRNSHLCFVHTVPWDQLFRNPHQALLHSANRPEDECAGEGLACYPLCAHGHCWGPGPTQCVNCSQFLRGQECVEECRVLQGLPREYVKDRFCLPCHPECQPQNGSVTCLGSEADQCVACAHYKDPPFCVARCPSGVKPDLSFMPIWKFADEEGACQPCPINCTHSPVTSIIAAVVGILLVVVVGLVLGILIKRRRQKIRKYTM

SEQ ID NO: 21 - protein, 687 aa, Canis lupus
MELAAWCRWGLLLALLPSGAAGTQVCTGTDMKLRLPASPETHLDMLRHLYQGCQVVQGNLELTYLPANASLSFLQDIQEVQGYVLIAHSQVRQIPLQRLRIVRGTQLFEDNYALAVLDNGDPLEGGIPAPGAAPGGLRELQLRSLTEILKGGVLIQRSPQLCHQDTILWKDVFHKNNQLALTLIDTNRSRACPPCSPACKDAHCWGASSGDCQSLTRTVCAGGCARCKGPQPTDCCHEQCAAGCTGPKHSDCLACLHFNHSGICELHCPALVTYNTDTFESMPNPEGRYTFGASCVTSCPYNYLSTDVGSCTLVCPLNNQEVTAEDGTQRCEKCSKPCARVCYGLGMEHLREVRAVTSANIQEFAGCKKIFGSLAFLPESFEGDPASNTAPLQPEQLRVFEALEEITGYLYISAWPDSLPNLSVFQNLRVIRGRVLHDGAYSLTLQGLGISWLGLRSLRELGSGLALIHRNARLCFVHTVPWDQLFRNPHQALLHSANRPEEECVGEGLACYPLCAHGHCWGPGPTQCVNCSQFLRGQECVEECRVLQGLPREYVKDRYCLPCHSECQPQNGSVTCFGSEADQCVACAHYKDPPFCVARCPSGVKPDLSFMPIWKFADEEGTCQPCPINCTHSCADLDEKGCPAEQRASPVTSIIAAVVGILLAVVVGLVLGILIKRRRQKIRKYTM

SEQ ID NO: 22 - protein, 687 aa, synthetic construct
  REGION 1..687: note = Dog (es)E2ectm; with substitutions A198V, R398Q, F425L, R473H,and T622A
MELAAWCRWGLLLALLPSGAAGTQVCTGTDMKLRLPASPETHLDMLRHLYQGCQVVQGNLELTYLPANASLSFLQDIQEVQGYVLIAHSQVRQIPLQRLRIVRGTQLFEDNYALAVLDNGDPLEGGIPAPGAAPGGLRELQLRSLTEILKGGVLIQRSPQLCHQDTILWKDVFHKNNQLALTLIDTNRSRACPPCSPVCKDAHCWGASSGDCQSLTRTVCAGGCARCKGPQPTDCCHEQCAAGCTGPKHSDCLACLHFNHSGICELHCPALVTYNTDTFESMPNPEGRYTFGASCVTSCPYNYLSTDVGSCTLVCPLNNQEVTAEDGTQRCEKCSKPCARVCYGLGMEHLREVRAVTSANIQEFAGCKKIFGSLAFLPESFEGDPASNTAPLQPEQLQVFEALEEITGYLYISAWPDSLPNLSVLQNLRVIRGRVLHDGAYSLTLQGLGISWLGLRSLRELGSGLALIHRNAHLCFVHTVPWDQLFRNPHQALLHSANRPEEECVGEGLACYPLCAHGHCWGPGPTQCVNCSQFLRGQECVEECRVLQGLPREYVKDRYCLPCHSECQPQNGSVTCFGSEADQCVACAHYKDPPFCVARCPSGVKPDLSFMPIWKFADEEGACQPCPINCTHSCADLDEKGCPAEQRASPVTSIIAAVVGILLAVVVGLVLGILIKRRRQKIRKYTM

SEQ ID NO: 23 - DNA, 2064 bp, Homo sapiens
  source 1..2064: mol_type = other DNA
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgtga

SEQ ID NO: 24 - DNA, 2064 bp, synthetic construct
  misc_feature 1..2064: note = sequence encoding h(es)E2ectm (human) with substitutions comparedto wild-type M198V, Q398R, F425L, H473R, and A622T
  source 1..2064: mol_type = other DNA
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccggtgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctcagagtgtttgagactctggaagagatcacaggttacctatacatctcagcatggccggacagcctgcctgacctcagcgtcctccagaacctgcaagtaatccggggacgaattctgcacaatggcgcctactcgctgaccctgcaagggctgggcatcagctggctggggctgcgctcactgagggaactgggcagtggactggccctcatccaccataacacccgcctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcacatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgtaa

SEQ ID NO: 25 - DNA, 2016 bp, Homo sapiens
  source 1..2016: mol_type = other DNA
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcccctctgacgtccatcatctctgcggtggttggcattctgctggtcgtggtcttgggggtggtctttgggatcctcatcaagcgacggcagcagaagatccggaagtacacgatgtaa

SEQ ID NO: 26 - DNA, 2064 bp, synthetic construct; identical to SEQ ID NO: 24
  misc_feature 1..2064: note = Sequence encoding Human (es)E2ectm-delta 16; with substitutionsM198V, Q398R, F425L, H473R, and A622T
  source 1..2064: mol_type = other DNA

SEQ ID NO: 27 - DNA, 2064 bp, Felis catus
  source 1..2064: mol_type = other DNA
atggagctggcggcctggtgccgctgggggctcctcctcgccctcctgccctccggagccacgggcacccaagtgtgcaccggcacagacatgaagctgcggctcccagccagtcccgagacccacctggacatgctccgccacctctaccagggctgtcaagtggtacagggcaacctggagctcacctacctgcatgccaatgccagcctctccttcctgcaggatatccaggaggtgcaaggctatgtgctcattgcccacaaccaagtgaaacaggtcccactgcagaggctacgaatcgtgcgaggcacccagctctttgaggacaactacgccctggccgtgctggacaacggagacccactggacagtggcacccctgctacaggggctgccctaggagggctgcgggagctgcagctccgaagcctcacagagatcctgaagggaggggtcctcattcagcggaacccgcagctctgccaccaggacacgattctgtggaaggacatcttccacaagaacaaccagctggccctcatgctgatagacaccaaccgctctcgggcctgccaaccctgttctccagcttgtaaagactcccactgctggggagcaagttccggggactgtcagagcttgactcgaactgtctgtgctggcggctgtgcccgctgcaagggcccgcagcccaccgactgctgccacgagcaatgtgctgctggctgcacgggccccaagcattctgactgcctggcctgcctccacttcaaccacagtggcatctgtgagctgcactgcccagccctggtcacctacaacacggacaccttcgaatccatgcccaaccctgagggccgttataccttcggtgccagctgtgtgactgcctgtccctacaactacctgtctacggacgtgggatcctgcaccctggtctgtcccctgaacaaccaagaggtgacagctgaggatggaacacagcggtgtgagaaatgcagcaagccctgtgcccgagtgtgctacggcctaggcatggagcacctgcgggaggcgagggcagtcaccagtgccaacatccaagaatttgtcggctgcaagaagatctttgggagcctggcgtttctgccagagagctttgagggggacccagcctccaacactgcccccctgcagcctgagcagctcagagtgtttgaggctctggaggagattacaggttacctgtacatctcagcgtggccagacagcttgcctaacctcagtgtcttccagaacctcagagtgatccggggccgagttctgcatgacggtgcttactcgctgacccttcaagggctgggcatcagctggctggggctgcgctcgctgcgggagctgggcagtgggctggccctcatccaccgcaactcccgcctctgcttcgtacacacggtgccctgggaccagctcttccggaacccccaccaggccctgctccacagcgccaaccggccagaggacgagtgcgcgggtgagggcctggcctgctatccgctgtgtgcccacgggcactgctggggtccgggacccacccagtgtgtcaactgcagccagttccttcggggccaggagtgcgtggaggaatgccgagtattgcaggggcttccccgggagtatgtgaaggataggttctgtctgccatgccacccggagtgtcagccccagaatggctcagtgacctgcttgggctcggaagctgaccagtgtgtggcctgtgcccactacaaggaccctcctttctgtgtggctcgctgccccagtggggtgaaacctgacctctccttcatgcccatctggaagttcgcagatgaggagggcacgtgccagccatgccccatcaactgcacccactcctgtgcggacctggacgagaagggctgccccgccgagcagagagccagccctgtgacgtccatcattgctgctgtggtgggcattctgctggtcgtggttgtggggctggtccttggcatcctaatcaagcgaaggcggcagaagatccggaagtacacgatgtga

SEQ ID NO: 28 - DNA, 2199 bp, synthetic construct
  misc_feature 1..2199: note = Feline (es)ERBB2ectm; with substitutions c728t, t729g, a1327c,g1328a, a1329g, t1408c, g1553a, a1999g, g2001c
  source 1..2199: mol_type = other DNA
gtacaagaatgaagttgtggagctgagagtcccctgcgtcgtgccccgagagccgagcagagctcccaggcagccgcccggcccttcgcagcccggtccagcccgagccatggggccggagccgcagtgagcaccatggagctggcggcctggtgccgctgggggctcctcctcgccctcctgccctccggagccacgggcacccaagtgtgcaccggcacagacatgaagctgcggctcccagccagtcccgagacccacctggacatgctccgccacctctaccagggctgtcaagtggtacagggcaacctggagctcacctacctgcatgccaatgccagcctctccttcctgcaggatatccaggaggtgcaaggctatgtgctcattgcccacaaccaagtgaaacaggtcccactgcagaggctacgaatcgtgcgaggcacccagctctttgaggacaactacgccctggccgtgctggacaacggagacccactggacagtggcacccctgctacaggggctgccctaggagggctgcgggagctgcagctccgaagcctcacagagatcctgaagggaggggtcctcattcagcggaacccgcagctctgccaccaggacacgattctgtggaaggacatcttccacaagaacaaccagctggccctcatgctgatagacaccaaccgctctcgggcctgccaaccctgttctccagtgtgtaaagactcccactgctggggagcaagttccggggactgtcagagcttgactcgaactgtctgtgctggcggctgtgcccgctgcaagggcccgcagcccaccgactgctgccacgagcaatgtgctgctggctgcacgggccccaagcattctgactgcctggcctgcctccacttcaaccacagtggcatctgtgagctgcactgcccagccctggtcacctacaacacggacaccttcgaatccatgcccaaccctgagggccgttataccttcggtgccagctgtgtgactgcctgtccctacaactacctgtctacggacgtgggatcctgcaccctggtctgtcccctgaacaaccaagaggtgacagctgaggatggaacacagcggtgtgagaaatgcagcaagccctgtgcccgagtgtgctacggcctaggcatggagcacctgcgggaggcgagggcagtcaccagtgccaacatccaagaatttgtcggctgcaagaagatctttgggagcctggcgtttctgccagagagctttgagggggacccagcctccaacactgcccccctgcagcctgagcagctccaggtgtttgaggctctggaggagattacaggttacctgtacatctcagcgtggccagacagcttgcctaacctcagtgtcctccagaacctcagagtgatccggggccgagttctgcatgacggtgcttactcgctgacccttcaagggctgggcatcagctggctggggctgcgctcgctgcgggagctgggcagtgggctggccctcatccaccgcaactcccacctctgcttcgtacacacggtgccctgggaccagctcttccggaacccccaccaggccctgctccacagcgccaaccggccagaggacgagtgcgcgggtgagggcctggcctgctatccgctgtgtgcccacgggcactgctggggtccgggacccacccagtgtgtcaactgcagccagttccttcggggccaggagtgcgtggaggaatgccgagtattgcaggggcttccccgggagtatgtgaaggataggttctgtctgccatgccacccggagtgtcagccccagaatggctcagtgacctgcttgggctcggaagctgaccagtgtgtggcctgtgcccactacaaggaccctcctttctgtgtggctcgctgccccagtggggtgaaacctgacctctccttcatgcccatctggaagttcgcagatgaggagggcgcctgccagccatgccccatcaactgcacccactcctgtgcggacctggacgagaagggctgccccgccgagcagagagccagccctgtgacgtccatcattgctgctgtggtgggcattctgctggtcgtggttgtggggctggtccttggcatcctaatcaagcgaaggcggcagaagatccggaagtacacgatgtaa

SEQ ID NO: 29 - DNA, 2151 bp, Felis catus
  source 1..2151: mol_type = other DNA
gtacaagaatgaagttgtggagctgagagtcccctgcgtcgtgccccgagagccgagcagagctcccaggcagccgcccggcccttcgcagcccggtccagcccgagccatggggccggagccgcagtgagcaccatggagctggcggcctggtgccgctgggggctcctcctcgccctcctgccctccggagccacgggcacccaagtgtgcaccggcacagacatgaagctgcggctcccagccagtcccgagacccacctggacatgctccgccacctctaccagggctgtcaagtggtacagggcaacctggagctcacctacctgcatgccaatgccagcctctccttcctgcaggatatccaggaggtgcaaggctatgtgctcattgcccacaaccaagtgaaacaggtcccactgcagaggctacgaatcgtgcgaggcacccagctctttgaggacaactacgccctggccgtgctggacaacggagacccactggacagtggcacccctgctacaggggctgccctaggagggctgcgggagctgcagctccgaagcctcacagagatcctgaagggaggggtcctcattcagcggaacccgcagctctgccaccaggacacgattctgtggaaggacatcttccacaagaacaaccagctggccctcatgctgatagacaccaaccgctctcgggcctgccaaccctgttctccagtgtgtaaagactcccactgctggggagcaagttccggggactgtcagagcttgactcgaactgtctgtgctggcggctgtgcccgctgcaagggcccgcagcccaccgactgctgccacgagcaatgtgctgctggctgcacgggccccaagcattctgactgcctggcctgcctccacttcaaccacagtggcatctgtgagctgcactgcccagccctggtcacctacaacacggacaccttcgaatccatgcccaaccctgagggccgttataccttcggtgccagctgtgtgactgcctgtccctacaactacctgtctacggacgtgggatcctgcaccctggtctgtcccctgaacaaccaagaggtgacagctgaggatggaacacagcggtgtgagaaatgcagcaagccctgtgcccgagtgtgctacggcctaggcatggagcacctgcgggaggcgagggcagtcaccagtgccaacatccaagaatttgtcggctgcaagaagatctttgggagcctggcgtttctgccagagagctttgagggggacccagcctccaacactgcccccctgcagcctgagcagctccaggtgtttgaggctctggaggagattacaggttacctgtacatctcagcgtggccagacagcttgcctaacctcagtgtcctccagaacctcagagtgatccggggccgagttctgcatgacggtgcttactcgctgacccttcaagggctgggcatcagctggctggggctgcgctcgctgcgggagctgggcagtgggctggccctcatccaccgcaactcccacctctgcttcgtacacacggtgccctgggaccagctcttccggaacccccaccaggccctgctccacagcgccaaccggccagaggacgagtgcgcgggtgagggcctggcctgctatccgctgtgtgcccacgggcactgctggggtccgggacccacccagtgtgtcaactgcagccagttccttcggggccaggagtgcgtggaggaatgccgagtattgcaggggcttccccgggagtatgtgaaggataggttctgtctgccatgccacccggagtgtcagccccagaatggctcagtgacctgcttgggctcggaagctgaccagtgtgtggcctgtgcccactacaaggaccctcctttctgtgtggctcgctgccccagtggggtgaaacctgacctctccttcatgcccatctggaagttcgcagatgaggagggcgcctgccagccatgccccatcaactgcacccactcccctgtgacgtccatcattgctgctgtggtgggcattctgctggtcgtggttgtggggctggtccttggcatcctaatcaagcgaaggcggcagaagatccggaagtacacgatgtaa

SEQ ID NO: 30 - DNA, 2061 bp, Canis lupus
  source 1..2061: mol_type = other DNA
atggagctggcggcctggtgccgctgggggctccttctcgccctcctgccctccggagccgcgggcacccaagtgtgcaccggcacagacatgaagctccggctcccggccagtcccgagacccacctggatatgctccgccacctgtaccagggctgtcaagtggtacaggggaacctggagctcacttacctgcctgccaatgccagcctgtccttcctgcaggatatccaggaggtgcagggctatgtgctcattgctcacagccaagtgaggcagatcccactgcagaggctacgaattgtgcgaggcacccagctctttgaggacaactacgccctggccgtgctggacaatggagacccgctggagggtggcatccctgcaccaggggcggcccaaggagggctgcgggagctgcagcttcgaagcctcacagagatcctgaagggaggggtcttgattcagcggagcccgcagctctgccaccaggacacgattttatggaaggacgtcttccataagaacaaccagctggccctcacgctgatagacaccaaccgcttttcggcctgcccgccctgttctccagcttgtaaagacgcccactgctggggggccagctccggggactgtcagagcttgacgcggactgtctgtgccgggggctgtgcccgctgcaagggcccacaacccaccgactgctgccacgagcagtgtgctgctggctgcacgggccccaagcactctgactgcctggcctgccttcacttcaaccacagtggcatctgtgagctgcactgcccagccctggtcacctacaacacggacaccttcgaatccatgcccaaccctgagggccgatataccttcggggccagctgtgtgacctcctgtccctacaactacctgtctacggatgtgggatcctgcaccctggtctgtcccctgaacaaccaagaggtgacggctgaggatgggacacagcggtgcgagaaatgcagcaagccctgtgcccgagtgtgctacggtctgggcatggagcacctgcgagaggtgagagcggtcaccagtgcgaacatccaggagtttgccggctgcaagaagatctttggaagcctggcatttttgccagagagctttgatggggacccagcctccaacactgcccccctacagcctgagcagctcagagtgtttgaggctctggaggagatcacaggttacctgtacatctcagcgtggccagacagcctgcctaacctcagtgtcttccagaacctgcgagtaatccggggacgagttctgcatgatggtgcctactcgctgaccctgcaagggctgggcatcagctggctggggctgcgctcgctgcgggaactgggcagtgggctggccctcatccaccgcaacgcccgcctttgcttcgtgcacacggtgccctgggaccagctcttccggaacccccaccaggccctgctccatagtgccaaccggccagaggaggagtgcgtgggcgagggcctggcctgctacccctgtgcccatgggcactgctggggtccagggcccacccagtgcgtcaactgcagccaattcctccggggccaggagtgcgtggaggaatgccgagtactgcaggggctgccccgagagtatgtgaaggacaggtactgtctaccgtgccactcagagtgtcagccccagaatggctcagtgacctgtttcggatcggaggctgaccagtgtgtggcctgcgcccactacaaggaccctcccttctgtgtggctcgctgccccagtggtgtgaaacctgacctgtccttcatgcccatctggaagttcgcagatgaggagggcacttgccagccgtgccccatcaactgcacccactcctgtgcggacctggacgagaagggctgtcccgccgagcagagagccagccctgtgacatccatcattgccgctgtggtgggcattctgctggctgtggtcgtggggctggtcctcggcatcctgatcaagcgaaggcggcagaagatccggaagtacactatgtga

SEQ ID NO: 31 - DNA, 2061 bp, synthetic construct
  misc_feature 1..2061: note = Canine (es)E2ectm; with substitutions c593t, t594g, a1192c,g1193a, a1194g, t1273c, g1418a, a1861g, t1863c
  source 1..2061: mol_type = other DNA
atggagctggcggcctggtgccgctgggggctccttctcgccctcctgccctccggagccgcgggcacccaagtgtgcaccggcacagacatgaagctccggctcccggccagtcccgagacccacctggatatgctccgccacctgtaccagggctgtcaagtggtacaggggaacctggagctcacttacctgcctgccaatgccagcctgtccttcctgcaggatatccaggaggtgcagggctatgtgctcattgctcacagccaagtgaggcagatcccactgcagaggctacgaattgtgcgaggcacccagctctttgaggacaactacgccctggccgtgctggacaatggagacccgctggagggtggcatccctgcaccaggggcggcccaaggagggctgcgggagctgcagcttcgaagcctcacagagatcctgaagggaggggtcttgattcagcggagcccgcagctctgccaccaggacacgattttatggaaggacgtcttccataagaacaaccagctggccctcacgctgatagacaccaaccgcttttcggcctgcccgccctgttctccagtgtgtaaagacgcccactgctggggggccagctccggggactgtcagagcttgacgcggactgtctgtgccgggggctgtgcccgctgcaagggcccacaacccaccgactgctgccacgagcagtgtgctgctggctgcacgggccccaagcactctgactgcctggcctgccttcacttcaaccacagtggcatctgtgagctgcactgcccagccctggtcacctacaacacggacaccttcgaatccatgcccaaccctgagggccgatataccttcggggccagctgtgtgacctcctgtccctacaactacctgtctacggatgtgggatcctgcaccctggtctgtcccctgaacaaccaagaggtgacggctgaggatgggacacagcggtgcgagaaatgcagcaagccctgtgcccgagtgtgctacggtctgggcatggagcacctgcgagaggtgagagcggtcaccagtgcgaacatccaggagtttgccggctgcaagaagatctttggaagcctggcatttttgccagagagctttgatggggacccagcctccaacactgcccccctacagcctgagcagctccaggtgtttgaggctctggaggagatcacaggttacctgtacatctcagcgtggccagacagcctgcctaacctcagtgtcctccagaacctgcgagtaatccggggacgagttctgcatgatggtgcctactcgctgaccctgcaagggctgggcatcagctggctggggctgcgctcgctgcgggaactgggcagtgggctggccctcatccaccgcaacgcccacctttgcttcgtgcacacggtgccctgggaccagctcttccggaacccccaccaggccctgctccatagtgccaaccggccagaggaggagtgcgtgggcgagggcctggcctgctacccctgtgcccatgggcactgctggggtccagggcccacccagtgcgtcaactgcagccaattcctccggggccaggagtgcgtggaggaatgccgagtactgcaggggctgccccgagagtatgtgaaggacaggtactgtctaccgtgccactcagagtgtcagccccagaatggctcagtgacctgtttcggatcggaggctgaccagtgtgtggcctgcgcccactacaaggaccctcccttctgtgtggctcgctgccccagtggtgtgaaacctgacctgtccttcatgcccatctggaagttcgcagatgaggagggcgcctgccagccgtgccccatcaactgcacccactcctgtgcggacctggacgagaagggctgtcccgccgagcagagagccagccctgtgacatccatcattgccgctgtggtgggcattctgctggctgtggtcgtggggctggtcctcggcatcctgatcaagcgaaggcggcagaagatccggaagtacactatgtga

SEQ ID NO: 32 - DNA, 2013 bp, Canis lupus
  source 1..2013: mol_type = other DNA
atggagctggcggcctggtgccgctgggggctccttctcgccctcctgccctccggagccgcgggcacccaagtgtgcaccggcacagacatgaagctccggctcccggccagtcccgagacccacctggatatgctccgccacctgtaccagggctgtcaagtggtacaggggaacctggagctcacttacctgcctgccaatgccagcctgtccttcctgcaggatatccaggaggtgcagggctatgtgctcattgctcacagccaagtgaggcagatcccactgcagaggctacgaattgtgcgaggcacccagctctttgaggacaactacgccctggccgtgctggacaatggagacccgctggagggtggcatccctgcaccaggggcggcccaaggagggctgcgggagctgcagcttcgaagcctcacagagatcctgaagggaggggtcttgattcagcggagcccgcagctctgccaccaggacacgattttatggaaggacgtcttccataagaacaaccagctggccctcacgctgatagacaccaaccgcttttcggcctgcccgccctgttctccagtgtgtaaagacgcccactgctggggggccagctccggggactgtcagagcttgacgcggactgtctgtgccgggggctgtgcccgctgcaagggcccacaacccaccgactgctgccacgagcagtgtgctgctggctgcacgggccccaagcactctgactgcctggcctgccttcacttcaaccacagtggcatctgtgagctgcactgcccagccctggtcacctacaacacggacaccttcgaatccatgcccaaccctgagggccgatataccttcggggccagctgtgtgacctcctgtccctacaactacctgtctacggatgtgggatcctgcaccctggtctgtcccctgaacaaccaagaggtgacggctgaggatgggacacagcggtgcgagaaatgcagcaagccctgtgcccgagtgtgctacggtctgggcatggagcacctgcgagaggtgagagcggtcaccagtgcgaacatccaggagtttgccggctgcaagaagatctttggaagcctggcatttttgccagagagctttgatggggacccagcctccaacactgcccccctacagcctgagcagctccaggtgtttgaggctctggaggagatcacaggttacctgtacatctcagcgtggccagacagcctgcctaacctcagtgtcctccagaacctgcgagtaatccggggacgagttctgcatgatggtgcctactcgctgaccctgcaagggctgggcatcagctggctggggctgcgctcgctgcgggaactgggcagtgggctggccctcatccaccgcaacgcccacctttgcttcgtgcacacggtgccctgggaccagctcttccggaacccccaccaggccctgctccatagtgccaaccggccagaggaggagtgcgtgggcgagggcctggcctgctacccctgtgcccatgggcactgctggggtccagggcccacccagtgcgtcaactgcagccaattcctccggggccaggagtgcgtggaggaatgccgagtactgcaggggctgccccgagagtatgtgaaggacaggtactgtctaccgtgccactcagagtgtcagccccagaatggctcagtgacctgtttcggatcggaggctgaccagtgtgtggcctgcgcccactacaaggaccctcccttctgtgtggctcgctgccccagtggtgtgaaacctgacctgtccttcatgcccatctggaagttcgcagatgaggagggcgcctgccagccgtgccccatcaactgcacccactcccctgtgacatccatcattgccgctgtggtgggcattctgctggctgtggtcgtggggctggtcctcggcatcctgatcaagcgaaggcggcagaagatccggaagtacactatgtga

SEQ ID NO: 33 - DNA, 20 bp, synthetic construct
  misc_feature 1..20: note = PCR primer (reverse) for human ERBB2-delta16
  source 1..20: mol_type = other DNA
ggagtgggtgcagttgatgg

SEQ ID NO: 34 - DNA, 20 bp, synthetic construct
  misc_feature 1..20: note = PCR primer (forward) for human ERBB2-delta16
  source 1..20: mol_type = other DNA
cctctgacgtccatcatctc

SEQ ID NO: 35 - DNA, 20 bp, synthetic construct
  misc_feature 1..20: note = PCR primer (reverse) for feline ERBB2-delta16
  source 1..20: mol_type = other DNA
ggagtgggtgcagttgatgg

SEQ ID NO: 36 - DNA, 19 bp, synthetic construct
  misc_feature 1..19: note = PCR primer (forward) for feline ERBB2-delta16
  source 1..19: mol_type = other DNA
cctgtgacgtccatcattg

SEQ ID NO: 37 - DNA, 20 bp, synthetic construct
  misc_feature 1..20: note = PCR primer (reverse) for canine ERBB2-delta16
  source 1..20: mol_type = other DNA
ggagtgggtgcagttgatgg

SEQ ID NO: 38 - DNA, 19 bp, synthetic construct
  misc_feature 1..19: note = PCR primer (forward) for canineERBB2-delta16
  source 1..19: mol_type = other DNA
cctgtgacatccatcattg

SEQ ID NO: 39 - protein, 15 aa, synthetic construct
  REGION 1..15: note = Human HER2 fragment
LPESFDGDPASNTAP

SEQ ID NO: 40 - protein, 15 aa, synthetic construct
  REGION 1..15: note = Human HER2 fragment
KIFGSLAPLPESFDG

SEQ ID NO: 41 - protein, 15 aa, synthetic construct
  REGION 1..15: note = Human HER2 fragment
SLAFLPESFDGDPAS

SEQ ID NO: 42 - protein, 15 aa, synthetic construct
  REGION 1..15: note = Human HER2 fragment
FDGDPASNTAPLQPE

SEQ ID NO: 43 - protein, 15 aa, synthetic construct
  REGION 1..15: note = Human HER2 fragment
PASNTAPLQPEQLQV

SEQ ID NO: 44 - protein, 11 aa, synthetic construct
  REGION 1..11: note = Human HER2 fragment
ESFDGDPASNT

(19) United States
(12) Patent Application Publication
(10) Pub. No.: US 2024/0252659 A1
(43) Pub. Date: Aug. 1, 2024

(54) METHODS AND IMMUNOGENIC COMPOSITIONS RELATING TO HER2 WITH SELECTIVE SEQUENCE MODIFICATIONS

(71) Applicant: Wayne State University, Detroit, MI (US)

(72) Inventors: Wei-Zen Wei, Grosse Pointe Farms, MI (US); Richard F. Jones (Deceased), Fayetteville, NY (US); Joyce Reyes, Rochester Hills, MI (US); Heather Gibson, Madison Heights, MI (US)

(21) Appl. No.: 18/528,165

(22) Filed: Dec. 4, 2023

Related U.S. Application Data
(63) Continuation of application No. 16/704,679, filed on Dec. 5, 2019, now abandoned.
(60) Provisional application No. 62/775,613, filed on Dec. 5, 2018.

(51) Int. Cl. A61K 47/58; A61K 31/337; A61K 35/76; A61K 38/28; A61K 39/00; A61K 39/39; A61K 47/54; A61K 47/69; C07C 227/16; C07C 229/22; C07K 16/28; C08F 120/36; C08G 81/02; C12N 15/86

(52) U.S. Cl. CPC A61K 47/58; A61K 31/337; A61K 35/76; A61K 38/28; A61K 39/001106; A61K 39/39; A61K 47/543; A61K 47/544; A61K 47/6909; A61K 47/6915; A61K 47/6921; C07C 227/16; C07C 229/22; C08F 120/36; C08G 81/027; A61K 39/001104; A61K 2039/585; C07K 16/2863; C12N 15/86

(57) ABSTRACT

Only limited success has been previously achieved from cancer vaccines targeting unmodified tumor-associated self-antigens and new compositions and methods are needed. Immunogenic compositions and methods of use thereof are provided according to the present disclosure which include a protein effective to stimulate immune activity against a tumor-associated self-antigen, or a variant thereof which is a tumor-associated self-antigen.

### REFERENCE TO RELATED APPLICATIONS

This application is a continuation of U.S. patent application Ser. No. 16/704,679, filed Dec. 5, 2019, which claims priority to U.S. Provisional Patent Application Ser. No. 62/775,613, filed Dec. 5, 2018, the entire content of each application is incorporated herein by reference.

### GRANT REFERENCE

This invention was made with government support under Grant No. RO1 CA076340, awarded by the National Institutes of Health. The Government has certain rights in the invention.

### SEQUENCE LISTING

The instant application contains a Sequence Listing which has been submitted electronically as a file in XML format and is hereby incorporated by reference in its entirety. Said XML format file, created on Apr. 17, 2024 is named Updated seq list 47WAY13703NA.xml and is 84,707 bytes in size.

### FIELD OF THE INVENTION

According to general aspects, the present invention relates to methods for generating immunogenic compositions to treat a disorder in a subject. In specific aspects, the present invention relates to immunogenic compositions which stimulate immune activity against a tumor-associated self-antigen, overcoming self-tolerance, and yet which substantially maintain the native structure of the tumor-associated self-antigen.

### BACKGROUND OF THE INVENTION

Increasingly, cancers are found to express proteins which have a non-oncogenic function in normal cells but which play a role in the development of a cancer, typically when overexpressed by the cancer cells.

Clinical successes of checkpoint inhibitors have demonstrated that endogenous immunity can destroy tumors. Although some tumor infiltrating lymphocytes (TIL) recognize neoantigens, the majority of TIL clones recognized tumor-associated self-antigens (TAA). Additionally, active vaccination targeting known TAA may create a favorable tumor microenvironment for neoantigen priming to enhance immune protection. Only limited success, however, has been achieved from cancer vaccines targeting unmodified TAA, specifically, the greatest challenge remains in the balance between self-tolerance and tumor immunity.

HER2 is a member of the epidermal growth factor receptor family described in detail in Harari D, et al., Oncogene, 2001, 19:6102-14. HER2 is overexpressed in breast, ovarian, non-small cell lung, endometrial, gastric and other cancers, stimulating cancer cell growth, see Harari D, et al., Oncogene, 2001, 19:6102-14. Some patients have pre-existing endogenous HER2 immunity, supporting the immunogenic nature of this non-mutated tumor-associated self-antigen (TAA), see Taylor C, et al., Clin. Cancer Res., 2007, 13:5133-43; and Moasser M M, Oncogene, 2007, 26:6469-87.

Both humoral and cellular HER2 immunity contribute to tumor growth inhibition, whether by direct killing through antibodies or T cells, or by Ab-dependent cell-mediated cytotoxicity, culminating in a comprehensive, multi-effector anti-tumor response.

There is a continuing need for compositions and methods for prevention and inhibition of cancer cells. In particular, there is a continuing need for compositions and methods relating to immunogenic compositions which stimulate immune activity against tumor-associated self-antigens. Further, there is a continuing need for methods of generating an immunogenic composition which stimulates immune activity against tumor-associated self-antigens, overcomes self-tolerance, yet substantially maintains the native structure of the tumor-associated self-antigen.

### SUMMARY OF THE INVENTION

Immunogenic compositions are provided according to the present invention which include a protein effective to stimulate immune activity against a tumor-associated self-antigen, or a variant thereof which is a tumor-associated self-antigen.

Immunogenic compositions are provided according to the present invention which include a protein effective to stimulate immune activity against HER2, a tumor-associated self-antigen, or a variant thereof which is a tumor-associated self-antigen.

Immunogenic compositions are provided according to the present invention which include a protein effective to stimulate immune activity against human HER2, a tumor-associated self-antigen, or a variant thereof which is a tumor-associated self-antigen.

According to aspects of the present invention, immunogenic compositions are provided which include a protein effective to stimulate immune activity against human HER2 of SEQ ID NO:1, a tumor-associated self-antigen.

According to aspects of the present invention, immunogenic compositions are provided which include a protein effective to stimulate immune activity against a variant of human HER2 of SEQ ID NO:1, a tumor-associated self-antigen.

According to aspects of the present invention, immunogenic compositions are provided which include a protein effective to stimulate immune activity against human HER2 of SEQ ID NO:16, a tumor-associated self-antigen.

According to aspects of the present invention, immunogenic compositions are provided which include a protein effective to stimulate immune activity against a variant human HER2 of SEQ ID NO:16, a tumor-associated self-antigen.

According to aspects of the present invention, immunogenic compositions are provided which include an immunogenic tumor-associated self-antigen characterized by one or more of: 1) effectiveness to stimulate immune activity against a specified tumor-associated self-antigen in a subject, 2) effectiveness to overcome self-tolerance of the specified tumor-associated self-antigen, and 3) substantial similarity to the native three dimensional structure of the specified tumor-associated self-antigen.

According to aspects of the present invention, immunogenic compositions are provided which include an immunogenic HER2 characterized by one or more of: 1) effectiveness to stimulate immune activity against HER2 in a subject, 2) effectiveness to overcome self-tolerance of HER2, and 3) substantial similarity to the native three-dimensional structure of HER2.

According to aspects of the present invention, immunogenic compositions are provided which include an immunogenic human HER2 characterized by one or more of: 1) effectiveness to stimulate immune activity against human HER2 in a subject, 2) effectiveness to overcome self-tolerance of human HER2, and 3) substantial similarity to the native three-dimensional structure of human HER2.

According to aspects of the present invention, immunogenic compositions are provided which include an immunogenic human HER2 of SEQ ID NO:1 characterized by one or more of: 1) effectiveness to stimulate immune activity against human HER2 of SEQ ID NO:1 in a subject, 2) effectiveness to overcome self-tolerance of human HER2 of SEQ ID NO:1, and 3) substantial similarity to the native three dimensional structure of human HER2 of SEQ ID NO:1.

According to aspects of the present invention, immunogenic compositions are provided which include an immunogenic variant of the human HER2 of SEQ ID NO:1 characterized by one or more of: 1) effectiveness to stimulate immune activity against the variant of human HER2 of SEQ ID NO:1 in a subject, 2) effectiveness to overcome self-tolerance of the variant of the human HER2 of SEQ ID NO:1, and 3) substantial similarity to the native three-dimensional structure of the variant of the human HER2 of SEQ ID NO:1.

According to aspects of the present invention, immunogenic compositions are provided which include an immunogenic human HER2 of SEQ ID NO:17 characterized by one or more of: 1) effectiveness to stimulate immune activity against human HER2 of SEQ ID NO:16 in a subject, 2) effectiveness to overcome self-tolerance of the human HER2 of SEQ ID NO:16, and 3) substantial similarity to the native three-dimensional structure of the human HER2 of SEQ ID NO:16.

According to aspects of the present invention, immunogenic compositions are provided which include an immunogenic variant of human HER2 of SEQ ID NO:17 characterized by one or more of: 1) effectiveness to stimulate immune activity against the variant of human HER2 of SEQ ID NO:16 in a subject, 2) effectiveness to overcome self-tolerance of the variant of the human HER2 of SEQ ID NO:16, and 3) substantial similarity to the native three-dimensional structure of the variant of the human HER2 of SEQ ID NO:16.

The term “substantial similarity” used herein in reference to a “native three-dimensional structure” of a protein indicates that the protein included in the immunogenic composition has at least some of the three-dimensional structural characteristics of the corresponding native protein such as, but not limited to, structural similarity evidenced by one or more of: 1) immunoassays using antibodies which recognize both the native protein structure and the protein of the immunogenic composition, 2) structural similarity evidenced by a percent amino acid sequence identity over the full-length of the the native protein structure and the protein of the immunogenic composition, wherein the percent amino acid sequence identity is at least 90%, at least 91%, at least 92%, at least 93%, at least 94%, at least 95%, at least 96%, at least 97%, at least 908%, at least 99%, or greater, 3) structural similarity evidenced by NMR spectroscopy, 4) structural similarity evidenced by X-ray crystallography, and 5) structural similarity evidenced by functional assay.

According to aspects of the present invention, immunogenic compositions are provided which include a protein which has, or includes, the amino acid sequence of SEQ ID NO:2.

According to aspects of the present invention, immunogenic compositions are provided which include a protein which has, or includes, the amino acid sequence of SEQ ID NO:2, and wherein the composition includes a pharmaceutically acceptable carrier.

According to aspects of the present invention, immunogenic compositions are provided which include a protein which has, or includes, the amino acid sequence of SEQ ID NO:2, and wherein the composition includes an adjuvant.

According to aspects of the present invention, immunogenic compositions are provided which include a protein which has, or includes, the amino acid sequence of SEQ ID NO:2, and wherein the composition includes an immunostimulating adjuvant.

Recombinant expression constructs are provided according to aspects of the present invention which include a nucleic acid encoding an immunogenic composition, wherein the immunogenic composition includes a protein which has, or includes, the amino acid sequence of SEQ ID NO:2, and wherein the nucleic acid encoding the immunogenic composition is operably linked to a heterologous regulatory nucleic acid sequence.

Recombinant expression constructs are provided according to aspects of the present invention which include a nucleic acid encoding an immunogenic composition, wherein the immunogenic composition includes a protein which has, or includes, the amino acid sequence of SEQ ID NO:2, wherein the nucleic acid encoding the immunogenic composition is operably linked to a heterologous regulatory nucleic acid sequence, and wherein the heterologous regulatory nucleic acid sequence includes a promoter.

Host cells are provided according to aspects of the present invention which include a recombinant expression construct, wherein the recombinant expression construct includes a nucleic acid encoding an immunogenic composition, wherein the immunogenic composition includes a protein which protein has, or includes, the amino acid sequence of SEQ ID NO:2, and wherein the nucleic acid encoding the immunogenic composition is operably linked to a heterologous regulatory nucleic acid sequence.

Host cells are provided according to aspects of the present invention which include a recombinant expression construct, wherein the recombinant expression construct includes a nucleic acid encoding an immunogenic composition, wherein the immunogenic composition includes a protein which has, or includes, the amino acid sequence of SEQ ID NO:2, wherein the nucleic acid encoding the immunogenic composition is operably linked to a heterologous regulatory nucleic acid sequence, and wherein the heterologous regulatory nucleic acid sequence includes a promoter.

Host cells are provided according to aspects of the present invention which include a recombinant expression construct in an expression vector, wherein the recombinant expression construct includes a nucleic acid encoding an immunogenic composition, wherein the immunogenic composition includes a protein which has, or includes, the amino acid sequence of SEQ ID NO:2, and wherein the nucleic acid encoding the immunogenic composition is operably linked to a heterologous regulatory nucleic acid sequence.

Host cells are provided according to aspects of the present invention which include a recombinant expression construct in an expression vector, wherein the recombinant expression construct includes a nucleic acid encoding an immunogenic composition, wherein the immunogenic composition includes a protein which has, or includes, the amino acid sequence of SEQ ID NO:2, wherein the nucleic acid encoding the immunogenic composition is operably linked to a heterologous regulatory nucleic acid sequence, and wherein the heterologous regulatory nucleic acid sequence includes a promoter.

Methods of treatment or prevention of a HER2+ cancer in a subject are provided according to aspects of the present invention which include administering a therapeutically effective amount of an immunogenic composition to the subject, wherein the immunogenic composition includes a protein which has, or includes, the amino acid sequence of SEQ ID NO:2.

Methods of treatment or prevention of a HER2+ cancer in a subject are provided according to aspects of the present invention which include administering a therapeutically effective amount of an immunogenic composition to the subject, wherein the immunogenic composition includes a protein which has, or includes, the amino acid sequence of SEQ ID NO:2, and wherein the composition includes a pharmaceutically acceptable carrier.

Methods of treatment or prevention of a HER2+ cancer in a subject are provided according to aspects of the present invention which include administering a therapeutically effective amount of an immunogenic composition to the subject, wherein the immunogenic composition includes a protein which has, or includes, the amino acid sequence of SEQ ID NO:2, and wherein the composition includes an adjuvant.

Methods of treatment or prevention of a HER2+ cancer in a subject are provided according to aspects of the present invention which include administering a therapeutically effective amount of an immunogenic composition to the subject, wherein the immunogenic composition includes a protein a protein which has, or includes, the amino acid sequence of SEQ ID NO:2, and wherein the composition includes an immunostimulating adjuvant.

Methods of treatment or prevention of a HER2+ cancer in a subject are provided according to aspects of the present invention which include administering a therapeutically effective amount of an immunogenic composition to the subject, wherein the immunogenic composition includes an expression construct encoding a protein which has, or includes, the amino acid sequence of SEQ ID NO:2. According to aspects of the present invention, the expression construct includes a nucleic acid encoding the amino acid sequence of SEQ ID NO:2 operably linked to a heterologous regulatory nucleic acid sequence.

Methods of treatment or prevention of a HER2+ cancer in a subject are provided according to aspects of the present invention which include administering a therapeutically effective amount of an immunogenic composition to the subject, wherein the immunogenic composition includes an expression construct encoding a protein which has, or includes, the amino acid sequence of SEQ ID NO:2. According to aspects of the present invention, the expression construct includes a nucleic acid encoding the amino acid sequence of SEQ ID NO:2 operably linked to a heterologous regulatory nucleic acid sequence, wherein the heterologous regulatory nucleic acid sequence is a promoter.

Methods of treatment or prevention of a HER2+ cancer in a subject are provided according to aspects of the present invention which include administering a therapeutically effective amount of an immunogenic composition to the subject, wherein the immunogenic composition includes an expression vector including an expression construct, the expression construct encoding a protein which has, or includes, the amino acid sequence of SEQ ID NO:2

Methods of treatment or prevention of a HER2+ cancer in a subject are provided according to aspects of the present invention which include administering a therapeutically effective amount of an immunogenic composition to the subject, wherein the immunogenic composition includes an expression vector, wherein the expression vector includes an expression construct encoding a protein which has, or includes, the amino acid sequence of SEQ ID NO:2. According to aspects of the present invention, the expression construct includes a nucleic acid encoding the amino acid sequence of SEQ ID NO:2 operably linked to a heterologous regulatory nucleic acid sequence.

Methods of treatment or prevention of a HER2+ cancer in a subject are provided according to aspects of the present invention which include administering a therapeutically effective amount of an immunogenic composition to the subject, wherein the immunogenic composition includes an expression vector, wherein the expression vector includes an expression construct encoding a protein which has, or includes, the amino acid sequence of SEQ ID NO:2. According to aspects of the present invention, the expression construct includes a nucleic acid encoding the amino acid sequence of SEQ ID NO:2 operably linked to a heterologous regulatory nucleic acid sequence, wherein the heterologous regulatory nucleic acid sequence is a promoter.

Methods of generating an immunogenic composition effective to stimulate immune activity against a tumor-associated self-antigen are provided according to aspects of the present invention which include: identifying a reference sequence; identifying at least a first amino acid sequence homologous to the reference sequence, wherein the reference sequence and the first amino acid sequence homologous to the reference sequence are not identical; comparing the homologous amino acid sequence and the reference sequence to identify at least a first difference between the homologous amino acid sequence and the reference sequence at a first position in the reference sequence and a corresponding first position in the homologous amino acid sequence; assigning a BLOSUM62 score to the first difference between the homologous amino acid sequence and the reference sequence, wherein the score represents a probability of substitution of an amino acid at the first position of the reference sequence with the amino acid at the corresponding first position in the homologous sequence, wherein the score falls within a numerical probability range of −4 to +3, where −4 is a number which indicates an extremely non-conservative substitution of the amino acid at the first position of the reference sequence with the amino acid at the corresponding first position of the homologous sequence such that the occurrence of substitution of the extremely non-conservative substitution is relatively rare or unlikely, where +3 is a number which indicates an extremely conservative substitution of the amino acid at the first position of the reference sequence with the amino acid at the corresponding first position of the homologous sequence such that the occurrence of substitution of the extremely non-conservative substitution is relatively frequent or likely, where a BLOSUM score of 0 indicates neutrality such that the occurrence of substitution has an equal probability; and synthesizing a new amino acid sequence identical to the reference amino acid sequence with the proviso that that the new amino acid sequence has at least one substitution at the first position with an amino acid present at the corresponding first position in the homologous amino acid sequence where the score assigned to the first difference is in the range of 0 to 1 and indicates neutrality such that the occurrence of substitution has an equal probability, thereby generating an immunogenic composition.

Methods of generating an immunogenic composition effective to stimulate immune activity against a tumor-associated self-antigen are provided according to aspects of the present invention which include: identifying a reference sequence; identifying at least 2, at least 3, at least 4, at least 5, at least 6, at least 7, at least 8, at least 9, at least 10, at least 11, at least 12, at least 13, at least 14, or at least 15, amino acid sequences homologous to the reference sequence and comparing them with the reference amino acid sequence, wherein the reference sequence and the sequences homologous to the reference sequence are not identical; comparing the homologous amino acid sequences and the reference sequence to identify at least a first difference between at least two or more of the homologous amino acid sequences and the reference sequence at a first position in the reference sequence and a corresponding first position in the homologous amino acid sequences; assigning a BLOSUM62 score to the first difference between the homologous amino acid sequences and the reference sequence, wherein the score represents a probability of substitution of an amino acid at the first position of the reference sequences with the amino acid at the corresponding first position in the homologous sequence, wherein the score falls within a numerical probability range of −4 to +3, where −4 is a number which indicates an extremely non-conservative substitution of the amino acid at the first position of the reference sequence with the amino acid at the corresponding first position of the homologous sequences such that the occurrence of substitution of the extremely non-conservative substitution is relatively rare or unlikely, where +3 is a number which indicates an extremely conservative substitution of the amino acid at the first position of the reference sequence with the amino acid at the corresponding first position of the homologous sequences such that the occurrence of substitution of the extremely non-conservative substitution is relatively frequent or likely, where a BLOSUM score of 0 indicates neutrality such that the occurrence of substitution has an equal probability; and synthesizing a new amino acid sequence identical to the reference amino acid sequence with the proviso that that the new amino acid sequence has at least one substitution at the first position with an amino acid present at the corresponding first position in the homologous amino acid sequences where the score assigned to the first difference is in the range of 0 to 1 and indicates neutrality such that the occurrence of substitution has an equal probability, thereby generating an immunogenic composition.

Methods of generating an immunogenic composition effective to stimulate immune activity against a tumor-associated self-antigen are provided according to aspects of the present invention which include: identifying a reference sequence; identifying at least a first amino acid sequence homologous to the reference sequence, wherein the reference sequence and the first amino acid sequence homologous to the reference sequence are not identical; comparing the homologous amino acid sequence and the reference sequence to identify at least 2, at least 3, at least 4, at least 5, at least 6, at least 7, at least 8, at least 9, at least 10, at least 11, at least 12, at least 13, at least 14, at least 15, or more, differences between the homologous amino acid sequence and the reference sequence at corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence and corresponding positions in the homologous amino acid sequence, assigning a BLOSUM score to the at least 2, at least 3, at least 4, at least 5, at least 6, at least 7, at least 8, at least 9, at least 10, at least 11, at least 12, at least 13, at least 14, at least 15, or more, differences between the homologous amino acid sequence and the reference sequence at corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence and corresponding positions in the homologous amino acid, wherein the BLOSUM score represents a probability of substitution of an amino acid at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions of the reference sequence with the amino acid at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous sequence, wherein the score falls within a numerical probability range of −4 to +3, where −4 is a number which indicates an extremely non-conservative substitution of the amino acid at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions of the reference sequence with the amino acid at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions of the homologous sequence such that the occurrence of substitution of the extremely non-conservative substitution is relatively rare or unlikely, where +3 is a number which indicates an extremely conservative substitution of the amino acid at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions of the reference sequence with the amino acid at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions of the homologous sequence such that the occurrence of substitution of the extremely non-conservative substitution is relatively frequent or likely, where a BLOSUM score of 0 indicates neutrality such that the occurrence of substitution has an equal probability; and synthesizing a new amino acid sequence identical to the reference amino acid sequence with the proviso that that the new amino acid sequence has at least one substitution at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions with an amino acid present at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous amino acid sequence where the score assigned to the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions is in the range of 0 to 1 and indicates neutrality such that the occurrence of substitution has an equal probability, thereby generating an immunogenic composition effective to stimulate immune activity against a tumor-associated self-antigen, effective to overcome self-tolerance of the tumor-associated self-antigen, and characterized by substantial similarity to the native three dimensional structure of the tumor-associated self-antigen.

According to aspects of the present invention, at least 2, at least 3, at least 4, at least 5, at least 6, at least 7, at least 8, at least 9, at least 10, at least 11, at least 12, at least 13, at least 14, or at least 15, substitutions are made in the reference amino acid sequence at 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence with an amino acid present at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous amino acid sequence where the BLOSUM score assigned to the differences identified at the 2^{nd}, 3^{rd}, 4^{th}, 5^{th}, 6^{th}, 7^{th}, 8^{th}, 9^{th}, 10^{th}, 11^{th}, 12^{th}, 13th, 14^{th}, 15^{th} or more, positions is in the range of 0 to 1.

Methods of generating an immunogenic composition effective to stimulate immune activity against a tumor-associated self-antigen are provided by the present invention which include identifying a reference sequence; identifying at least a first amino acid sequence homologous to the reference sequence, wherein the reference sequence and the first amino acid sequence homologous to the reference sequence are not identical; comparing the homologous amino acid sequence and the reference sequence to identify at least a first difference between the homologous amino acid sequence and the reference sequence at a first position in the reference sequence and a corresponding first position in the homologous amino acid sequence; assigning a score to the first difference between the homologous amino acid sequence and the reference sequence, wherein the score represents a probability of substitution of an amino acid at the first position of the reference sequence with the amino acid at the corresponding first position in the homologous sequence, wherein the score falls within a numerical probability range of x to y, where x is a number which indicates an extremely non-conservative substitution of the amino acid at the first position of the reference sequence with the amino acid at the corresponding first position of the homologous sequence such that the occurrence of substitution of the extremely non-conservative substitution is relatively rare or unlikely, where y is a number which indicates an extremely conservative substitution of the amino acid at the first position of the reference sequence with the amino acid at the corresponding first position of the homologous sequence such that the occurrence of substitution of the extremely non-conservative substitution is relatively frequent or likely, where a number intermediate between x and y indicates neutrality such that the occurrence of substitution has an equal probability; synthesizing a new amino acid sequence identical to the reference amino acid sequence with the proviso that that the new amino acid sequence has at least one substitution at the first position with an amino acid present at the corresponding first position in the homologous amino acid sequence where the score assigned to the first difference is intermediate between x and y indicates neutrality such that the occurrence of substitution has an equal probability, thereby generating an immunogenic composition.

According to aspects of inventive methods, at least 2, at least 3, at least 4, at least 5, at least 6, at least 7, at least 8, at least 9, at least 10, at least 11, at least 12, at least 13, at least 14, or at least 15, amino acid sequences homologous to the reference sequence are identified and compared with the reference amino acid sequence.

According to aspects of inventive methods, at least 2, at least 3, at least 4, at least 5, at least 6, at least 7, at least 8, at least 9, at least 10, at least 11, at least 12, at least 13, at least 14, at least 15, or more, differences between the homologous amino acid sequence and the reference sequence at corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence and a corresponding position in the homologous amino acid sequence are identified and assigned a score.

According to aspects of inventive methods, at least 2, at least 3, at least 4, at least 5, at least 6, at least 7, at least 8, at least 9, at least 10, at least 11, at least 12, at least 13, at least 14, or at least 15, substitutions are made in the reference amino acid sequence at 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence with an amino acid present at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous amino acid sequence where the score assigned to the differences identified at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions is intermediate between x and y indicating that the occurrence of substitution has an equal probability.

### BRIEF DESCRIPTION OF THE DRAWINGS

FIGS. 1A and 1B generally show aspects of HER2 vaccines and BLOSUM scores.

FIG. 1A is a schematic diagram showing HER2 ECD subdomains I-IV and mAbs reactive to each subdomain. Signal peptide (SP) and transmembrane domain (TM), are indicated.

FIG. 1B shows graphs of BLOSUM62 scores plotted for amino acid residues of indicated HER2 vaccines; for amino acid substitutions, BLOSUM scores are calculated relative to human HER2 (top row). pE2TM is wt HER2 (amino acid residues 1-687). pE2Neu contains wt HER2 SP and subdomains I & II (residues 1-390), fused to rat Neu (residues 394-691), with AEF inserted into subdomain III. pNeuE2 is the converse of pE2Neu, containing Neu SP and subdomains I & II, fused to HER2 (residues 391-687).

FIG. 1C is a graph showing results in BALB HER2 Tg mice which received three DNA electrovaccinations with pE2TM, pNeu, the hybrid vaccines pE2Neu, pNeuE2, or admixed pE2TM and pNeu. The levels of anti-Neu or anti-HER2 Ab in the immune sera were measured by flow cytometry. There were 6-9 mice per group.

FIGS. 2Ai, 2Aii, 2B, 2C, 2D, 2E, 2F, and 2G generally show human HER2 point mutants: stability and immunogenicity in vitro and in vivo.

FIG. 2Ai is an image showing single residue substitutions depicted on the DNA scheme of human HER2.

FIG. 2Aii is an image showing single residue substitutions depicted on the space-filling model (RCSB 2A91, JSmol viewer) of human HER2.

FIG. 2B is a series of graphs showing results of expression of recombinant point mutants; test vaccines were expressed in NIH 3T3 cells, then analyzed by flow cytometry using Ab4, Ab5, and N12 as detection antibodies.

FIG. 2C shows immunogenicity of point mutants tested in BALB/c wild-type (wt) mice after one electrovaccination. Antibody response was monitored 2 weeks post-vaccination.

FIGS. 2D, 2E, 2F, and 2G generally show immunogenicity of selected point mutations in BALB HER-2 or B6 HER-2 transgenic (Tg) mice.

FIG. 2D shows results obtained when mice were electrovaccinated twice with pE2TM, pE2Neu, pE2TM-Q141K, or pE2TM-Q429R. In BALB HER-2 Tg mice, HER2 antibody levels were measured by flow cytometry following each vaccination and IFN-γ producing SC were measured after two vaccinations using 3T3/EKB cells as the APC (with 3T3/KB as controls). There were 4-6 mice per group. *p<0.05

FIG. 2E shows results obtained when mice were electrovaccinated twice with pE2TM, pE2Neu, pE2TM-Q141K, or pE2TM-Q429R. In B6 HER-2 Tg mice, Treg were depleted 10 days prior to vaccination. HER2 Ab levels were measured by flow cytometry while IFNγ-producing SC were measured with the APC TC-1/E2, with TC-1 as controls. There were 4-6 mice per group. *p<0.05

FIG. 2F shows results obtained when mice were electrovaccinated twice with pE2TM, pE2Neu, pE2TM-Q141K, or pE2TM-Q429R. In BALB HER-2 Tg mice, HER2 antibody levels were measured by flow cytometry following each vaccination and IFN-γ producing SC were measured after two vaccinations using 3T3/EKB cells as the APC (with 3T3/KB as controls). There were 4-6 mice per group. *p<0.05

FIG. 2G shows results obtained when mice were electrovaccinated twice with pE2TM, pE2Neu, pE2TM-Q141K, or pE2TM-Q429R. In B6 HER-2 Tg mice, Treg were depleted 10 days prior to vaccination. HER2 Ab levels were measured by flow cytometry while IFNγ-producing SC were measured with the APC TC-1/E2, with TC-1 as controls. There were 4-6 mice per group. *p<0.05

FIGS. 3A, 3B, 3C, 3D, and 3E generally show evolution-selected HER2 vaccine and the corresponding immune response.

FIGS. 3A and 3B are schematic diagrams in which positions of amino acid (AA) substitutions are depicted. Shaded substitutions are unique to rhesus monkey (XP_001090430). pE2TM is human HER2; ph(es)E2TM is pE2TM with the indicated 5 AA substitutions; prmE2TM is rhesus monkey HER2.

FIG. 3C is a set of graphs showing expression of recombinant proteins. 3T3 cells were transfected with ph(es)E2TM or prmE2TM and analyzed by flow cytometry. pE2TM and pVax blank vector were controls; positive cells are gated.

FIGS. 3D and 3E are graphs showing induction of HER2 specific response by the test vaccine constructs. BALB HER2 Tg mice were electrovaccinated twice with pE2TM, ph(es)E2TM or prmE2TM. HER2 specific antibody (Ab) (FIG. 3D) and IFN-γ producing SC were measured after the last vaccination. There were 4 mice per group. *p<0.05

FIG. 4A is a graph showing induction of HER2 immunity by ph(es)E2TM in BALB HER2Tg. BALB HER2Tg mice were electrovaccinated twice with pE2TM, pE2Neu, or ph(es)E2TM. HER2 Ab levels were measured.

FIG. 4B is a graph showing induction of HER2 immunity by ph(es)E2TM in Treg-depleted B6 HER2 Tg mice. Treg-depleted B6 HER2 Tg mice were electrovaccinated 2× with pE2TM, pE2Neu, or ph(es)E2TM and α-HER2 Ab measured 2 weeks after each vaccination.

FIG. 4C is a graph showing results from BALB HER2 Tg mice which were inoculated with D2F2/E2t tumors in the mammary fatpad and IFNγ-producing SC were analyzed 4 weeks post tumor inoculation. There were 6-9 mice per group. *p<0.05, **p<0.01, ***p<0.0001

FIG. 4D is a graph showing results from IFNγ-producing SC in B6 HER2 Tg which were evaluated 2 weeks post second vaccination, without tumor inoculation. There were 6-9 mice per group. *p<0.05, **p<0.01, ***p<0.0001

FIGS. 5A and 5B generally show HER2 immune serum epitope mapping. For these studies, immune serum was incubated on peptide microarray slide containing a library of 168 human HER2 15-mer peptides with 11-mer overlaps. Bound antibodies were detected with a fluorescence labeled anti-mouse IgG. Specific binding was expressed by the fold increase in mean pixel value for a particular peptide over the average pixel values of all peptides. A 2-fold increase is considered positive binding.

FIG. 5A shows the binding profile of immune sera from BALB HER2 and B6 HER2 Tg mice vaccinated with pVax, pE2TM, pE2Neu or ph(es)E2TM. p95 ₃₅₅LPESFDGDPASNTAP₃₆₉ (SEQ ID NO:40) was recognized most prominently by pE2Neu and ph(es)E2TM immune sera.

FIG. 5B is an image showing position of cognate peptide domain p95 indicated in the ribbon model of human HER2 (RCSB 2A91, JSmol viewer). Three extra residues AEF introduced during pE2Neu construction are located between residues 368-369 within p95.

FIGS. 6A and 6B generally show tumor growth inhibition in vitro and in vivo.

FIG. 6A is a graph showing inhibition of SK-BR-3 tumor cell proliferation in vitro. Cells were incubated with increasing concentrations of HER2 binding Ab from vaccinated BALB HER2 Tg mice shown in FIG. 4. The negative control was pVax immune serum; and the positive control was Gefitinib. Cell viability was measured by Alamar Blue assay. Values are means±SE from three independent samples, each in triplicate, and normalized to untreated cells. Statistical significance was determined by Student's t test.

FIG. 6B is a graph of results of tumor growth inhibition after BALB HER2Tg mice were vaccinated twice either with pE2TM, pE2Neu or ph(es)E2TM, at 2 week intervals. D2F2/E2t cells were injected intra-fat pad and tumor growth was monitored twice weekly. *p<0.05, **p<0.01, ***p<0.0001

FIG. 7 shows BLOSUM scores of amino acid substitutions in immunogenic HER2 vaccine compositions according to aspects of the present invention. BLOSUM62 scores are plotted for AA residues of indicated HER2 vaccine constructs: pE2TM is human native HER2ectm; ph(es)E2TM is pE2TM with the indicated 5 evolutionary-selected AA substitutions; prmE2TM is rhesus monkey HER2ectm, which differs from human E2ectm by 7 AA residues, as indicated. BLOSUM62 scores are calculated for substituted AA's in reference to human native E2TM.

FIG. 8 shows sequence comparisons and alignment results for the 14 protein sequences listed in Table 1: huE2ectm protein (SEQ ID NO:1), Bonobo-XP_008955 (SEQ ID NO:3), BolivSqMonkey-XP (SEQ ID NO:4), Chimp-XP_0033155 (SEQ ID NO:5), GoldSnNoMonkey-X (SEQ ID NO:6), Gorilla-XP_00404 (SEQ ID NO:7), GrnMonkey-XP_008 (SEQ ID NO:8), OliveBaboon-XP_0 (SEQ ID NO:9), RhesusErbB2-XP_0 (SEQ ID NO:10), Rhesus-HER2prot2 (SEQ ID NO:11), SumOrangutan-XP_ (SEQ ID NO:12), WhChGibbon-XP_00 (SEQ ID NO:13), WhTufEarMarmoset (SEQ ID NO:14), Macaca-XP_005584 (SEQ ID NO:15). For this alignment, global protein alignment against reference molecule was used, wherein the reference molecule as huE2ectm protein, Region 1 to 687. Fourteen sequences were aligned and the total length of aligned sequences with gaps was 688 amino acids. Parameters: Scoring matrix: BLOSUM 62. * indicates aa substitutions in hu vs. primate E2ectm [Blosum62 score] 198 3M->8V [1]; 398 7Q->6R [1]; 425 8F->6L [0]; 473 4H->10R [0]; and 622 2A->12T [0].

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, PA, 2003; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol Biol. 2002; 185, Humana Press; Current Protocols in Stem Cell Biology, ISBN: 9780470151808; Chu, E. and Devita, V. T., Eds., Physicians' Cancer Chemotherapy Drug Manual, Jones & Bartlett Publishers, 2005; J. M. Kirkwood et al., Eds., Current Cancer Therapeutics, 4th Ed., Current Medicine Group, 2001; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, PA: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

Immunogenic compositions are provided according to the present invention which include a protein effective to stimulate immune activity against HER2, human HER2, a tumor-associated self-antigen, or a variant of any thereof which is a tumor-associated self-antigen.

According to aspects of the present invention, immunogenic compositions are provided which include a protein effective to stimulate immune activity against human HER2 of SEQ ID NO:1, a tumor-associated self-antigen.

According to aspects of the present invention, immunogenic compositions are provided which include a protein effective to stimulate immune activity against a variant of human HER2 of SEQ ID NO:1 which is a tumor-associated self-antigen.

According to aspects of the present invention, immunogenic compositions are provided which include a protein effective to stimulate immune activity against human HER2 of SEQ ID NO:16, a tumor-associated self-antigen.

According to aspects of the present invention, immunogenic compositions are provided which include a protein effective to stimulate immune activity against a variant human HER2 of SEQ ID NO:16 which is a tumor-associated self-antigen.

Immunogenic compositions are provided according to aspects of the present invention which include a protein effective to stimulate immune activity against a non-human HER2 which is a tumor-associated self-antigen, such as a canine HER2 or feline HER2.

Immunogenic compositions are provided according to aspects of the present invention which include the protein of SEQ ID NO:2 which is characterized by 5 amino acid substitutions compared to the wild-type human HER2 protein of SEQ ID NO:1, namely, M198V, Q398R, F425L, H473R, and A622T. Optionally, one or more additional amino acids maybe added to the N-terminus, C-terminus, or both the N-terminus and C-terminus, such as 1, 2, 3, 4, 5, 6, 7, 8, 9, 10, 11, 12, 13, 14, 15, 16, 17, 18, 19, or 20 amino acids, with the proviso that the cytoplasmic domain of the wild-type HER2 is not present in an immunogenic composition of the present invention.

Immunogenic compositions are provided according to aspects of the present invention which include the protein of SEQ ID NO:17 which is characterized by 5 amino acid substitutions compared to the wild-type human HER2 protein of SEQ ID NO:1, namely, M198V, Q398R, F425L, H473R, and A622T. Optionally, one or more additional amino acids maybe added to the N-terminus, C-terminus, or both the N-terminus and C-terminus, such as 1, 2, 3, 4, 5, 6, 7, 8, 9, 10, 11, 12, 13, 14, 15, 16, 17, 18, 19, or 20 amino acids, with the proviso that the cytoplasmic domain of the wild-type HER2 is not present in an immunogenic composition of the present invention.

Immunogenic compositions are provided according to aspects of the present invention which include a protein having the amino acid sequence selected from the group consisting of: SEQ ID NO:2, SEQ ID NO:17, SEQ ID NO:19, and SEQ ID NO:22; or a variant of any thereof. Optionally, one or more additional amino acids maybe added to the N-terminus, C-terminus, or both the N-terminus and C-terminus, such as 1, 2, 3, 4, 5, 6, 7, 8, 9, 10, 11, 12, 13, 14, 15, 16, 17, 18, 19, or 20 amino acids, with the proviso that the cytoplasmic domain of the wild-type HER2 is not present in an immunogenic composition of the present invention.

According to aspects of the present invention, immunogenic compositions are provided which include an immunogenic tumor-associated self-antigen characterized by one or more of: 1) effectiveness to stimulate immune activity against a specified tumor-associated self-antigen in a subject, 2) effectiveness to overcome self-tolerance of the specified tumor-associated self-antigen, and 3) substantial similarity to the native three dimensional structure of the specified tumor-associated self-antigen.

According to aspects of the present invention, immunogenic compositions are provided which include an immunogenic HER2 characterized by one or more of: 1) effectiveness to stimulate immune activity against HER2 in a subject, 2) effectiveness to overcome self-tolerance of HER2, and 3) substantial similarity to the native three-dimensional structure of HER2.

According to aspects of the present invention, immunogenic compositions are provided which include an immunogenic human HER2 characterized by one or more of: 1) effectiveness to stimulate immune activity against human HER2 in a subject, 2) effectiveness to overcome self-tolerance of human HER2, and 3) substantial similarity to the native three-dimensional structure of human HER2.

According to aspects of the present invention, immunogenic compositions are provided which include an immunogenic human HER2 of SEQ ID NO:2 characterized by one or more of: 1) effectiveness to stimulate immune activity against human HER2 of SEQ ID NO:1 in a subject, 2) effectiveness to overcome self-tolerance of human HER2 of SEQ ID NO:1, and 3) substantial similarity to the native three dimensional structure of human HER2 of SEQ ID NO:1.

According to aspects of the present invention, immunogenic compositions are provided which include an immunogenic variant of the human HER2 of SEQ ID NO:2 characterized by one or more of: 1) effectiveness to stimulate immune activity against the variant of human HER2 of SEQ ID NO:1 in a subject, 2) effectiveness to overcome self-tolerance of the variant of the human HER2 of SEQ ID NO:1, and 3) substantial similarity to the native three-dimensional structure of the variant of the human HER2 of SEQ ID NO:1.

According to aspects of the present invention, immunogenic compositions are provided which include an immunogenic human HER2 of SEQ ID NO:17 characterized by one or more of: 1) effectiveness to stimulate immune activity against human HER2 of SEQ ID NO:16 in a subject, 2) effectiveness to overcome self-tolerance of the human HER2 of SEQ ID NO:16, and 3) substantial similarity to the native three-dimensional structure of the human HER2 of SEQ ID NO:16.

According to aspects of the present invention, immunogenic compositions are provided which include an immunogenic variant of human HER2 of SEQ ID NO:17 characterized by one or more of: 1) effectiveness to stimulate immune activity against the variant of human HER2 of SEQ ID NO:16 in a subject, 2) effectiveness to overcome self-tolerance of the variant of the human HER2 of SEQ ID NO:16, and 3) substantial similarity to the native three-dimensional structure of the variant of the human HER2 of SEQ ID NO:16.

As used herein, the term “variant” refers to a variation of a nucleic acid sequence, a variation of a nucleic acid sequence encoding a protein, or a variation of a protein in which one or more nucleotides or amino acid residues have been modified by nucleotide or amino acid substitution, addition, or deletion while retaining all, or at least some, of the function of the reference nucleic acid sequence or protein. Variants of a nucleic acid sequence or protein described herein are characterized by conserved functional properties compared to the corresponding nucleic acid sequence or protein.

Mutations can be introduced using standard molecular biology techniques, such as chemical synthesis, site-directed mutagenesis and PCR-mediated mutagenesis.

One of skill in the art will recognize that one or more amino acid mutations can be introduced without altering the functional properties of a desired protein. For example, one or more amino acid substitutions, additions, or deletions can be made without altering the functional properties of a desired protein.

Biological activity of a protein variant is readily determined by one of skill in the art, for instance using any of the functional assays described herein or other functional assays known in the art.

Variants of a protein described herein are characterized by conserved functional properties compared to the corresponding protein and have 75%, 80%, 85%, 90%, 95%, 96%, 97%, 98%, 99% or greater identity to the amino acid sequence of a reference protein.

Variants of SEQ ID NO:2 are provided according to aspects of the present invention in which M198 is substituted by any of: V, A, H, L, I, Q, or F; Q398 is substituted by any of: R, H, E, K, N, D, H, M, or S; F425 is substituted by any of: L, Y, W, I, or M; H473 is substituted by any of: R, Y, N, Q, or E; and A622 is substituted by any of: T, S, C, G, V, or M. Optionally, one or more additional amino acids maybe added to the N-terminus, C-terminus, or both the N-terminus and C-terminus, such as 1, 2, 3, 4, 5, 6, 7, 8, 9, 10, 11, 12, 13, 14, 15, 16, 17, 18, 19, or 20 amino acids, with the proviso that the cytoplasmic domain of the wild-type HER2 is not present in an immunogenic composition of the present invention.

Variants of SEQ ID NO:17 are provided according to aspects of the present invention in which M198 is substituted by any of: V, A, H, L, I, Q, or F; Q398 is substituted by any of: R, H, E, K, N, D, H, M, or S; F425 is substituted by any of: L, Y, W, I, or M; H473 is substituted by any of: R, Y, N, Q, or E; and A622 is substituted by any of: T, S, C, G, V, or M. Optionally, one or more additional amino acids maybe added to the N-terminus, C-terminus, or both the N-terminus and C-terminus, such as 1, 2, 3, 4, 5, 6, 7, 8, 9, 10, 11, 12, 13, 14, 15, 16, 17, 18, 19, or 20 amino acids, with the proviso that the cytoplasmic domain of the wild-type HER2 is not present in an immunogenic composition of the present invention.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

An immunogenic composition of the present invention can be administered to a subject alone or as part of a pharmaceutical composition. Inventive compositions are suitable for administration to subjects by a variety of routes including systemic and local routes of administration. Inventive compositions are suitable for administration to subjects by a variety of routes illustratively including intravenous, oral, parenteral, intramuscular, subcutaneous and mucosal. Inventive compositions are suitable for administration to subjects by a variety of routes illustratively including but not limited to, oral, rectal, nasal, pulmonary, epidural, ocular, otic, intraarterial, intracardiac, intracerebroventricular, intradermal, intravenous, intramuscular, intraperitoneal, intraosseous, intrathecal, intravesical, subcutaneous, topical, transdermal, and transmucosal, such as by sublingual, buccal, vaginal, and inhalational, routes of administration.

Optionally, an immunogenic composition according to aspects of the present invention includes a pharmaceutically acceptable carrier.

Optionally, an immunogenic composition according to aspects of the present invention includes an adjuvant.

The term “pharmaceutically acceptable” refers to a material which can be administered to a subject along with an inventive immunogenic composition without causing significant undesirable biological effects and without interacting in a deleterious manner with any other component of the immunogenic composition. An immunogenic composition including a pharmaceutically acceptable carrier is also termed a “pharmaceutical composition” herein.

Pharmaceutical compositions suitable for administration illustratively include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers; diluents; solvents; or vehicles include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of the required particle size in the case of dispersions, and by the use of surfactants.

Compositions suitable for injection optionally include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Pharmaceutical compositions according to the present invention may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of an injectable pharmaceutical form can be brought about by the use of agents delaying absorption, for example, aluminum monostearate and gelatin.

Further exemplary adjuvants include immunostimulating adjuvants such as Freund's complete adjuvant; Freund's incomplete adjuvant; aluminum hydroxide such as commercially available as Alhydrogel, Accurate Chemical & Scientific Co, Westbury, New York; and Gerbu adjuvant, available from C-C Biotech, Poway, California.

Solid dosage forms for oral administration include capsules, tablets, pills, powders, and granules. In such solid dosage forms, an inventive conjugate is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alginates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Microencapsulated formulations of inventive immunogenic compositions are also contemplated.

Liquid dosage forms for oral administration include pharmaceutically acceptable emulsions, solutions, suspensions, syrups, and elixirs. In addition to a conjugate according to the present invention, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, a pharmaceutical composition according to the present invention can also include adjuvants, such as wetting agents, emulsifying and suspending agents, sweetening, flavoring, and perfuming agents.

Suspensions, in addition to an inventive conjugate, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Further specific details of pharmaceutical formulation can be found in Pharmaceutical Dosage Forms: Tablets, eds. H. A. Lieberman et al., New York: Marcel Dekker, Inc., 1989; L. V. Allen, Jr. et al., Ansel's Pharmaceutical Dosage Forms and Drug Delivery Systems, 8th Ed., Philadelphia, PA, Lippincott, Williams & Wilkins, 2004; and Remington, The Science and Practice of Pharmacy, 21^{st} ed., Lippincott, Williams & Wilkins, Philadelphia, PA, 2006.

An inventive immunogenic composition is optionally delivered in conjunction with an additional therapeutic agent according to aspects of the present invention. A therapeutic agent suitable in this regard illustratively includes an analgesic, an antibiotic, an anti-inflammatory, an anti-cancer agent, an antiviral, a gamma or beta radiation emitting species, an enzyme, and a hormone. In addition, two or more additional therapeutic agents may be administered to a subject.

Recombinant expression constructs are provided according to aspects of the present invention which include a nucleic acid encoding a protein effective to stimulate immune activity against tumor-associated self-antigen, operably linked to a heterologous regulatory nucleic acid sequence.

Recombinant expression constructs are provided according to aspects of the present invention which include a nucleic acid encoding a protein effective to stimulate immune activity against HER2, operably linked to a heterologous regulatory nucleic acid sequence.

Recombinant expression constructs are provided according to aspects of the present invention which include a nucleic acid encoding a protein effective to stimulate immune activity against human HER2, operably linked to a heterologous regulatory nucleic acid sequence.

Recombinant expression constructs are provided according to aspects of the present invention which include a nucleic acid encoding a protein including the amino acid sequence of SEQ ID NO:2, or a variant thereof, operably linked to a heterologous regulatory nucleic acid sequence. According to aspects of the present invention, a nucleic acid encoding a protein including the amino acid sequence of SEQ ID NO:2 is the nucleic acid of SEQ ID NO:25

It is appreciated that due to the degenerate nature of the genetic code, alternate nucleic acid sequences encode a specified protein, and that such alternate nucleic acids may be expressed to produce the desired protein. Thus, variants of SEQ ID NO:25 which encode SEQ ID NO:2 are provided according to aspects of the present invention.

Recombinant expression constructs are provided according to aspects of the present invention which include a nucleic acid encoding a protein including the amino acid sequence of SEQ ID NO:17, or a variant thereof, operably linked to a heterologous regulatory nucleic acid sequence. According to aspects of the present invention, a nucleic acid encoding a protein including the amino acid sequence of SEQ ID NO:17 is the nucleic acid of SEQ ID NO:27

It is appreciated that due to the degenerate nature of the genetic code, alternate nucleic acid sequences encode a specified protein, and that such alternate nucleic acids may be expressed to produce the desired protein. Thus, variants of SEQ ID NO:27 which encode SEQ ID NO:17 are provided according to aspects of the present invention.

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide and is usually shown as the ordering of the sense strand.

The term “expression construct” is used herein to refer to a double-stranded recombinant DNA molecule containing a desired nucleic acid coding sequence for a protein to be expressed and containing one or more regulatory elements necessary or desirable for the expression of the operably linked coding sequence. The terms “expressed” and “expression” refer to transcription of a nucleic acid sequence to produce a corresponding mRNA and/or translation of the mRNA to produce the corresponding protein. Expression constructs can be generated recombinantly or by DNA synthesis using well-known methodology.

The term “recombinant” is used to indicate a nucleic acid construct in which two or more nucleic acids are linked and which are not found linked in nature.

The term “regulatory element” as used herein refers to a nucleotide sequence which controls some aspect of the expression of nucleic acid sequences. Exemplary regulatory elements illustratively include an enhancer, an internal ribosome entry site (IRES), an intron; an origin of replication, a polyadenylation signal (polyA), a promoter, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation.

Expression constructs operable to express a desired protein include, for example, in operable linkage: a promoter, a DNA sequence encoding a desired protein and a transcription termination site.

The term “operably linked” as used herein refers to a nucleic acid in functional relationship with a second nucleic acid.

A regulatory element included in an expression construct is a promoter in particular aspects.

The term “promoter” is well-known in the art and refers to one or more DNA sequences operably linked to a nucleic acid sequence to be transcribed and which bind an RNA polymerase and allow for initiation of transcription. A promoter is typically positioned upstream (5′) of a nucleic acid encoding a peptide or protein to be expressed.

An mRNA polyadenylation (pA) sequence may be included such as, but not limited to SV40-pA, beta-globin-pA and SCF-pA.

An expression construct may include sequences necessary for amplification in bacterial cells, such as a selection marker (e.g. kanamycin or ampicillin resistance gene) and a replicon.

An internal ribosome entry site (IRES) is an optionally included nucleic acid sequence that permits translation initiation at an internal site in an mRNA. IRES are well-known in the art, for example as described in Pelletier, J. et al., Nature, 334:320-325, 1988; Vagner, S. et al., EMBO Rep., 2:893-898, 2001; and Hellen, C. U. et al, Genes Dev. 15:1593-1612, 2001.

The term “transcription termination site” refers to a DNA sequence operable to terminate transcription by an RNA polymerase. A transcription termination site is generally positioned downstream (3′) of a nucleic acid encoding a peptide or protein to be expressed.

A leader sequence is optionally included in an expression construct.

An expression construct can be cloned into an expression vector for transformation into prokaryotic or eukaryotic cells and expression of the encoded peptides and/or protein(s). As used herein, “expression vectors” are defined as polynucleotides which, when introduced into an appropriate host cell or in a cell-free expression system, can be transcribed and translated, producing the encoded polypeptide(s).

Expression vectors are known in the art and include plasmids, cosmids, viruses and bacteriophages, for example. Expression vectors can be, without limitation, prokaryotic vectors, insect vectors, or eukaryotic vectors.

For example, an expression construct including, in operable linkage: a promoter, a DNA sequence encoding a desired protein and a transcription termination site, is included in a plasmid, cosmid, BAC, YAC, virus or bacteriophage expression vector. Particular viral vectors illustratively include those derived from adenovirus, adeno-associated virus and lentivirus.

Particular vectors are known in the art and one of skill in the art will recognize an appropriate vector for a specific purpose.

Host cells are provided according to aspects of the present invention which include a recombinant expression construct, wherein the recombinant expression construct includes a nucleic acid encoding an immunogenic composition, wherein the immunogenic composition includes an immunogenic tumor-associated self-antigen, and wherein the nucleic acid encoding the immunogenic composition is operably linked to a heterologous regulatory nucleic acid sequence.

Host cells are provided according to aspects of the present invention which include a recombinant expression construct, wherein the recombinant expression construct includes a nucleic acid encoding an immunogenic composition, wherein the immunogenic composition includes HER2, and wherein the nucleic acid encoding the immunogenic composition is operably linked to a heterologous regulatory nucleic acid sequence.

Host cells are provided according to aspects of the present invention which include a recombinant expression construct, wherein the recombinant expression construct includes a nucleic acid encoding an immunogenic composition, wherein the immunogenic composition includes human HER2, and wherein the nucleic acid encoding the immunogenic composition is operably linked to a heterologous regulatory nucleic acid sequence.

Host cells are provided according to aspects of the present invention which include a recombinant expression construct, wherein the recombinant expression construct includes a nucleic acid encoding an immunogenic composition, wherein the immunogenic composition includes the amino acid sequence of SEQ ID NO:2, or a variant thereof, and wherein the nucleic acid encoding the immunogenic composition is operably linked to a heterologous regulatory nucleic acid sequence.

Host cells are provided according to aspects of the present invention which include a recombinant expression construct, wherein the recombinant expression construct includes SEQ ID NO:25, or a variant thereof which encodes the amino acid sequence of SEQ ID NO:2, operably linked to a heterologous regulatory nucleic acid sequence.

Host cells are provided according to aspects of the present invention which include a recombinant expression construct, wherein the recombinant expression construct encodes the amino acid sequence of SEQ ID NO:17, or a variant thereof, operably linked to a heterologous regulatory nucleic acid sequence.

Host cells are provided according to aspects of the present invention which include a recombinant expression construct, wherein the recombinant expression construct includes SEQ ID NO:27, or a variant thereof which encodes the amino acid sequence of SEQ ID NO:17, operably linked to a heterologous regulatory nucleic acid sequence.

Any suitable expression vector/host cell system can be used for expression according to aspects of the present invention.

Expression of a desired protein using a recombinant expression vector is accomplished according to aspects of the present invention by introduction of the expression vector into a eukaryotic or prokaryotic host cell expression system such as an insect cell, mammalian cell, yeast cell, fungus, bird egg, bacterial cell or any other single or multicellular organism recognized in the art.

Host cells containing the recombinant expression vector are maintained under conditions wherein the desired protein is produced. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art.

For expression in a host cell, any of the well-known procedures for introducing recombinant nucleic acids into host cells may be used, such as calcium phosphate transfection, polybrene, protoplast fusion, electroporation, sonoporation, liposomes and microinjection, examples of which are described in Sambrook et al., Molecular Cloning: A Laboratory Manual, Cold Spring Harbor Laboratory Press, 2001; and Ausubel, F. et al., (Eds.), Current Protocols in Molecular Biology, 2014.

The host cell may be in vivo or in vitro.

According to particular aspects, “naked” nucleic acid, such as DNA or mRNA, is introduced into a host cell in vivo for expression in the host. Introduction of a nucleic acid encoding the desired protein to a cell in vivo can be accomplished by a variety of techniques, including, but not limited to, electroporation, sonoporation, liposome administration, injection and microinjection.

According to particular aspects, the nucleic acid encoding the protein is present in an expression vector and the expression vector is introduced into a host cell in vivo for expression in the host. Introduction of an expression vector including a nucleic acid encoding the desired protein to a cell in vivo can be accomplished by a variety of techniques, including, but not limited to, electroporation, sonoporation, liposome administration, injection, and microinjection. According to particular aspects, the expression vector is a virus, including, but not limited to an adenovirus, an adeno-associated virus, and a lentivirus.

According to particular aspects, the expression vector is a virus, cells are infected with the virus, and the infected cells are administered to the subject, whereby the protein is expressed in the cells in vivo. According to particular aspects, the expression vector is a virus, white blood cells are infected with the virus, and the infected white blood cells are administered to the subject, whereby the protein is expressed in the white blood cells in vivo. Optionally, the cells are derived from the subject, infected with the virus, and then administered to the subject, whereby the protein is expressed in the white blood cells in vivo.

Optionally, the expressed protein is isolated from the host cell, or where the protein is produced by other methods, such as by chemical synthesis, the protein is isolated from reagents, such as chemical synthesis reagents. The term “isolated” in this context refers to removal of the protein from other components of a host cell, or from chemical synthetic reagents, such that the isolated protein includes at least 20% by weight of the protein, at least 25% by weight of the protein, at least 30% by weight of the protein, at least 35% by weight of the protein, at least 40% by weight of the protein, at least 45% by weight of the protein, at least 50% by weight of the protein, at least 55% by weight of the protein, at least 60% by weight of the protein, at least 65% by weight of the protein, at least 70% by weight of the protein, at least 75% by weight of the protein, at least 80% by weight of the protein, at least 85% by weight of the protein, at least 90% by weight of the protein, at least 91% by weight of the protein, at least 92% by weight of the protein, at least 93% by weight of the protein, at least 94% by weight of the protein, at least 95% by weight of the protein, at least 96% by weight of the protein, at least 97% by weight of the protein, at least 98% by weight of the protein, at least 99% by weight of the protein, or greater % by weight of the protein.

Methods of treatment or prevention of a HER2+ cancer in a subject are provided according to aspects of the present invention which include administering a therapeutically effective amount of an immunogenic composition. According to aspects of the present invention, an immunogenic composition is administered which includes an immunogenic tumor-associated self-antigen characterized by one or more of: 1) effectiveness to stimulate immune activity against a specified tumor-associated self-antigen in a subject, 2) effectiveness to overcome self-tolerance of the specified tumor-associated self-antigen, and 3) substantial similarity to the native three dimensional structure of the specified tumor-associated self-antigen.

Methods of treatment or prevention of a HER2+ cancer in a subject are provided according to aspects of the present invention which include administering a therapeutically effective amount of an immunogenic composition including a protein, wherein the protein has, or includes, the amino acid sequence of SEQ ID NO:2.

Optionally, the immunogenic composition is administered as a nucleic acid encoding a protein including the amino acid sequence of SEQ ID NO:2 operably linked to a heterologous regulatory nucleic acid sequence, such as in an expression construct, expression vector, or as “naked” DNA or mRNA to be expressed in vivo in the subject.

Methods of treatment or prevention of a HER2+ cancer in a subject are provided according to aspects of the present invention which include administering a therapeutically effective amount of an immunogenic composition including a protein, wherein the protein has, or includes, a variant of the amino acid sequence of SEQ ID NO:2.

Optionally, the immunogenic composition is administered as a nucleic acid encoding a protein including a variant of the amino acid sequence of SEQ ID NO:2 operably linked to a heterologous regulatory nucleic acid sequence, such as in an expression construct, expression vector, or as “naked” DNA or mRNA to be expressed in vivo in the subject.

Methods of treatment or prevention of a HER2+ cancer in a subject are provided according to aspects of the present invention which include administering a therapeutically effective amount of an immunogenic composition including a protein, wherein the protein has, or includes, the amino acid sequence of SEQ ID NO:17.

Optionally, the immunogenic composition is administered as a nucleic acid encoding a protein including the amino acid sequence of SEQ ID NO:17 operably linked to a heterologous regulatory nucleic acid sequence, such as in an expression construct, expression vector, or as “naked” DNA or mRNA to be expressed in vivo in the subject.

Methods of treatment or prevention of a HER2+ cancer in a subject are provided according to aspects of the present invention which include administering a therapeutically effective amount of an immunogenic composition including a protein, wherein the protein has, or includes, a variant of the amino acid sequence of SEQ ID NO:17.

Optionally, the immunogenic composition is administered as a nucleic acid encoding a protein including a variant of the amino acid sequence of SEQ ID NO:17 operably linked to a heterologous regulatory nucleic acid sequence, such as in an expression construct, expression vector, or as “naked” DNA or mRNA to be expressed in vivo in the subject.

Optionally, a method of treatment or prevention of a HER2+ cancer in a subject according to aspects of the present invention further includes administering an additional therapeutic agent or treatment to the subject.

HER2+ cancers include, but are not limited to breast, ovarian, non-small cell lung, and gastric cancers. The term “HER2+” is used interchangeably with “HER2-positive” and refers to cancers in which cancer cells have higher than normal levels of HER2, see, for example, Slamon D J, et al., 1989, 244:707-712, PMID: 2470152. Further, HER2 signaling is dysregulated in some cancers—thus differing from normal cells, see for example, Ménard S, et al., J. Cell Physiol., 2000, 281:150-162, PMID: 10623878.

The dosage of an inventive pharmaceutical composition administered to a subject will vary based on factors such as the route of administration; the age, health, and weight of the subject to whom the composition is to be administered; the nature and extent of the subject's symptoms, if any, and the effect desired. Usually a daily dosage of an immunogenic composition is in the range of about 0.001 to 100 milligrams per kilogram of a subject's body weight. A daily dose may be administered as two or more divided doses to obtain the desired effect. An inventive pharmaceutical composition may also be formulated for sustained release to obtain desired results.

A subject treated according to methods and using compositions of the present invention can be mammalian or non-mammalian. A mammalian subject can be any mammal including, but not limited to, a human; a non-human primate; a rodent such as a mouse, rat, or guinea pig: a domesticated pet such as a cat or dog; a horse, cow, pig, sheep, goat, or rabbit. A non-mammalian subject can be any non-mammal including, but not limited to, a bird such as a duck, goose, chicken, or turkey. Subjects can be either gender and can be any age. In aspects of methods including administration of an inventive composition to a subject, the subject is human.

As used herein, the terms “treatment” or “treating” are used to refer to administration of an immunogenic composition for obtaining beneficial or desired results including clinically beneficial or desired results which include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The term “treatment” encompasses prophylactic treatment. As used herein, the term “prevent” refers to the reduction in the risk of acquiring or developing a given condition, or the reduction or inhibition of the recurrence or said condition in a subject.

Methods of generating an immunogenic composition are provided according to aspects of the present invention which include: identifying a reference sequence; identifying at least a first amino acid sequence homologous to the reference sequence, wherein the reference sequence and the first amino acid sequence homologous to the reference sequence are not identical; comparing the homologous amino acid sequence and the reference sequence to identify at least a first difference between the homologous amino acid sequence and the reference sequence at a first position in the reference sequence and a corresponding first position in the homologous amino acid sequence; assigning a BLOSUM62 score to the first difference between the homologous amino acid sequence and the reference sequence, wherein the score represents a probability of substitution of an amino acid at the first position of the reference sequence with the amino acid at the corresponding first position in the homologous sequence, wherein the score falls within a numerical probability range of −4 to +3, where −4 is a number which indicates an extremely non-conservative substitution of the amino acid at the first position of the reference sequence with the amino acid at the corresponding first position of the homologous sequence such that the occurrence of substitution of the extremely non-conservative substitution is relatively rare or unlikely, where +3 is a number which indicates an extremely conservative substitution of the amino acid at the first position of the reference sequence with the amino acid at the corresponding first position of the homologous sequence such that the occurrence of substitution of the extremely non-conservative substitution is relatively frequent or likely, where a BLOSUM score of 0 indicates neutrality such that the occurrence of substitution has an equal probability; and synthesizing a new amino acid sequence identical to the reference amino acid sequence with the proviso that that the new amino acid sequence has at least one substitution at the first position with an amino acid present at the corresponding first position in the homologous amino acid sequence where the score assigned to the first difference is in the range of 0 to 1 and indicates neutrality such that the occurrence of substitution has an equal probability, thereby generating an immunogenic composition. According to aspects, at least 2, at least 3, at least 4, at least 5, at least 6, at least 7, at least 8, at least 9, at least 10, at least 11, at least 12, at least 13, at least 14, or at least 15, amino acid sequences homologous to the reference sequence are identified and compared with the reference amino acid sequence. According to aspects, at least 2, at least 3, at least 4, at least 5, at least 6, at least 7, at least 8, at least 9, at least 10, at least 11, at least 12, at least 13, at least 14, at least 15, or more, differences between the homologous amino acid sequence and the reference sequence at corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence and a corresponding position in the homologous amino acid sequence are identified and assigned a BLOSUM score. According to aspects, at least 2, at least 3, at least 4, at least 5, at least 6, at least 7, at least 8, at least 9, at least 10, at least 11, at least 12, at least 13, at least 14, or at least 15, substitutions are made in the reference amino acid sequence at 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence with an amino acid present at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous amino acid sequence where the BLOSUM score assigned to the differences identified at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions is in the range of 0 to 1.

Methods of generating an immunogenic composition according to aspects of the present invention include: identifying a reference sequence; identifying at least a first amino acid sequence homologous to the reference sequence, wherein the reference sequence and the first amino acid sequence homologous to the reference sequence are not identical; comparing the homologous amino acid sequence and the reference sequence to identify at least a first difference between the homologous amino acid sequence and the reference sequence at a first position in the reference sequence and a corresponding first position in the homologous amino acid sequence; assigning a score to the first difference between the homologous amino acid sequence and the reference sequence, wherein the score represents a probability of substitution of an amino acid at the first position of the reference sequence with the amino acid at the corresponding first position in the homologous sequence, wherein the score falls within a numerical probability range of x to y, where x is a number which indicates an extremely non-conservative substitution of the amino acid at the first position of the reference sequence with the amino acid at the corresponding first position of the homologous sequence such that the occurrence of substitution of the extremely non-conservative substitution is relatively rare or unlikely, where y is a number which indicates an extremely conservative substitution of the amino acid at the first position of the reference sequence with the amino acid at the corresponding first position of the homologous sequence such that the occurrence of substitution of the extremely non-conservative substitution is relatively frequent or likely, where a number intermediate between x and y indicates neutrality such that the occurrence of substitution has an equal probability; and synthesizing a new amino acid sequence identical to the reference amino acid sequence with the proviso that that the new amino acid sequence has at least one substitution at the first position with an amino acid present at the corresponding first position in the homologous amino acid sequence where the score assigned to the first difference is intermediate between x and y indicates neutrality such that the occurrence of substitution has an equal probability, thereby generating an immunogenic composition. According to aspects, at least 2, at least 3, at least 4, at least 5, at least 6, at least 7, at least 8, at least 9, at least 10, at least 11, at least 12, at least 13, at least 14, or at least 15, amino acid sequences homologous to the reference sequence are identified and compared with the reference amino acid sequence. According to aspects, at least 2, at least 3, at least 4, at least 5, at least 6, at least 7, at least 8, at least 9, at least 10, at least 11, at least 12, at least 13, at least 14, at least 15, or more, differences between the homologous amino acid sequence and the reference sequence at corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence and a corresponding position in the homologous amino acid sequence are identified and assigned a score. According to aspects, at least 2, at least 3, at least 4, at least 5, at least 6, at least 7, at least 8, at least 9, at least 10, at least 11, at least 12, at least 13, at least 14, or at least 15, substitutions are made in the reference amino acid sequence at 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence with an amino acid present at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous amino acid sequence where the score assigned to the differences identified at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions is intermediate between x and y indicating that the occurrence of substitution has an equal probability.

Methods of generating an immunogenic composition according to aspects of the present invention, also referred to herein as “evolution selection” were used to design minimally modified antigens effective to stimulate immune activity against a tumor-associated self-antigen, effective to overcome self-tolerance of the tumor-associated self-antigen. Methods of generating an immunogenic composition described herein are grounded on principles of protein evolution and based on the premise that amino acids that tend to be frequently substituted with particular residues across the proteins of closely related animal species, such as, but not limited to, HER proteins, are not likely to alter the structure and function of the proteins. Amino acids at these commonly substituted positions are therefore good candidates for substitutions aimed at increasing the “foreignness” of the protein without altering the target epitopes. The degrees of conservatism at the commonly substituted positions can be analyzed by BLOSUM62 scoring or another suitable bioinformatic sequence alignment procedure. The substitutions are tested for immunogenicity and therapeutic effect in various in vitro and in vivo assays.

Methods of generating an immunogenic composition according to aspects of the present invention produce an immunogenic protein effective to stimulate immune activity against a tumor-associated self-antigen in a subject, administration of which is effective to overcome self-tolerance of the tumor-associated self-antigen, and which is characterized by substantial similarity to the native three dimensional structure of the tumor-associated self-antigen.

Methods of generating an immunogenic composition according to aspects of the present invention produce an immunogenic protein characterized by one or more of: 1) effectiveness to stimulate immune activity against a specified tumor-associated self-antigen in a subject, 2) effectiveness to overcome self-tolerance of the specified tumor-associated self-antigen, and 3) substantial similarity to the native three dimensional structure of the specified tumor-associated self-antigen.

Methods of generating an immunogenic composition according to aspects of the present invention produce an immunogenic protein characterized by one or more of: 1) effectiveness to stimulate immune activity against HER2 in a subject, 2) effectiveness to overcome self-tolerance of HER2, and 3) substantial similarity to the native three dimensional structure of HER2.

Methods of generating an immunogenic composition according to aspects of the present invention produce an immunogenic protein characterized by one or more of: 1) effectiveness to stimulate immune activity against human HER2 in a subject, 2) effectiveness to overcome self-tolerance of human HER2, and 3) substantial similarity to the native three dimensional structure of human HER2.

Methods of generating an immunogenic composition according to aspects of the present invention produce an immunogenic protein characterized by one or more of: 1) effectiveness to stimulate immune activity against human HER2 of SEQ ID NO:1 in a subject, 2) effectiveness to overcome self-tolerance of human HER2 of SEQ ID NO:1, and 3) substantial similarity to the native three dimensional structure of human HER2 of SEQ ID NO:1.

Methods of generating an immunogenic composition according to aspects of the present invention produce an immunogenic protein characterized by one or more of: 1) effectiveness to stimulate immune activity against a variant of human HER2 of SEQ ID NO:1 in a subject, 2) effectiveness to overcome self-tolerance of a variant of the human HER2 of SEQ ID NO:1, and 3) substantial similarity to the native three dimensional structure of a variant of the human HER2 of SEQ ID NO:1.

Methods of generating an immunogenic composition according to aspects of the present invention produce an immunogenic protein characterized by one or more of: 1) effectiveness to stimulate immune activity against human HER2 of SEQ ID NO:16 in a subject, 2) effectiveness to overcome self-tolerance of the human HER2 of SEQ ID NO:16, and 3) substantial similarity to the native three dimensional structure of the human HER2 of SEQ ID NO:16.

Methods of generating an immunogenic composition according to aspects of the present invention produce an immunogenic protein characterized by one or more of: 1) effectiveness to stimulate immune activity against a variant of human HER2 of SEQ ID NO:16 in a subject, 2) effectiveness to overcome self-tolerance of a variant of the human HER2 of SEQ ID NO:16, and 3) substantial similarity to the native three dimensional structure of a variant of the human HER2 of SEQ ID NO:16.

Methods of generating an immunogenic composition according to aspects of the present invention are useful to generate immunogenic compositions against any tumor-associated self-antigen where self-tolerance of the tumor-associated self-antigen must be overcome in order to treat and/or prevent cancer, including, but not limited to, HER2, HER1 (EGFR), and HER3.

Accordingly, a method according to aspects of the present invention for generating a minimally modified immunogenic polypeptide for breaking tolerance to a host's target HER family antigen begins with the step of comparing the amino acid sequence of the target HER family receptor to those of homologous HER family receptors of several species closely related to the host. One or more residues found frequently to be substituted by common residues in these related species are then substituted with the common residues into the host's target HER family receptor, to create at least one candidate antigen. Finally, it is determined whether the candidate antigen is capable of breaking tolerance to the target HER family antigen.

In exemplary embodiments of the invention, the target is human HER2; the homologous receptors are non-human primateHER2; and BLOSUM62 scoring is the means of evaluating the sites of substitution and the conservativeness of possible substitutions. The invention also includes designer antigens designed by the process of evolution selection, and vaccines including those antigens.

In vivo testing of one of the resulting designer HER2 vaccines (i.e. immunogenic compositions according to aspects of the present invention), which contains 5 substitutions in the extracellular domain (ECD), showed it induces elevated humoral and cellular immunity to HER2 and reduces tumor growth. The vaccine antigen h(es)E2ectm includes SEQ ID NO:2 in the sequence list found below. For reference, the wild type antigen includes the SEQ ID NO:1.

It has recently been determined that the delta 16 splice variant of HER2, which lacks exon 16, spontaneously dimerizes to cause constitutive stimulation and proliferation of epithelial cells (Wada R, et al., Mol. Med. Rep., 2016, 14(6):5104-5110. doi: 10.3892/mmr.2016.5892). For human HER2, the sequence of this splice variant is given as SEQ ID NO:16. The invention includes evolution selected variants of the delta 16 splice variant, such as human vaccine antigen Human (es)E2ectm-delta 16, SEQ ID NO:17.

The invention is not limited to human HER family vaccines. In fact, commercialization may be accomplished most rapidly for vaccines against cancers of domestic animals that are prone to breast cancer, such as cats and dogs. There are too few feline or canine ERBB2 sequences available for an analogous approach to designing (es)ERBB2 vaccines for those species, so the positions and patterns of aa substitutions from the human/primate alignment analysis were superimposed, as indicated in the headings for the feline vaccine antigen sequence (SEQ ID NO:19) (Feline (es)E2ectm), and the canine vaccine antigen sequence (SEQ ID NO:22) (Dog (es)E2ectm). The delta 16 variants of these antigens are given as SEQ ID NO:20 and SEQ ID NO:23, respectively. For reference, the wild type sequences are SEQ ID NO:18 and SEQ ID NO:21.

Exemplary cDNA sequences are also provided in the sequence list, for above-mentioned vaccine antigens, and for their wild type counterparts (SEQ ID NOs: 24-33). It will be understood that any DNA sequence that encodes the disclosed peptides of the vaccine antigens is encompassed by the present inventions.

Also provided in the sequence list, as SEQ ID NOs:34-39, are PCR primers for construction of human ERBB2-delta16, feline ERBB2-delta16 and canine ERBB2-delta16 and derivatives thereof. They can be used, for example, with New England Biolab's Q5 Site-Directed Mutagenesis procedure (NEB E0554) when cloned into circular expression vector (e.g., pVAX1).

The designer antigens of the present invention can be deployed in any conceivable vaccine composition. In exemplary embodiments, the antigens are encoded in naked DNA plasmids for expression in vivo. The antigens can alternatively be incorporated into an unlimited range of expression constructs, and delivered in an unlimited range of vectors. The antigens should also be effective as recombinant proteins, if introduced by an effective delivery system. The vaccines are contemplated at present as preventative therapeutic vaccines, and are also useful in a therapeutic setting, against existing tumors.

Embodiments of inventive compositions and methods are illustrated in the following examples. These examples are provided for illustrative purposes and are not considered limitations on the scope of inventive compositions and methods.

### EXAMPLES

### Materials and Methods

### Mice

Wild type C57BL/6 and BALB/c mice are purchased from Charles River Laboratory (Frederick, MD, US). Heterozygous C57BL/6 HER-2 Tg mice (B6 HER-2 Tg), which express the full-length, wild type human HER-2 under the whey acidic protein (WAP) promoter were generated as described in detail in Piechocki et al., J. Immunol. 2003, 171(11):5787-94 and maintained by mating with wild type B6 mice as described in detail in Piechocki et al., J. Immunol. 2003, 171(11):5787-94.

BALB/c HER-2 Tg (BALB HER-2 Tg) mice were generated by back-crossing B6 HER-2 Tg mice with wild type BALB/c mice (described in detail in Yong C S, et al., 2015, PLoS One. 10: e0136817. doi: 10.1371/journal.pone.0136817) and are maintained by mating with BALB/c mice. Transgene positive mice were identified by PCR as described in detail in Radkevich-Brown O, et al., 2009, Cancer Res. 69: 212-8. doi: 10.1158/0008-5472.CAN-083092. B6 HER2 Tg mice have been deposited at Jackson lab repository (B6.Cg-Tg(Wap-ERBB2)229Wzw/J).

### Construction and Validation of DNA Constructs

pVAX1 (Thermo Fisher Scientific) was used for constructing ion of each of the following genetic vaccines. pE2TM contains codons 1-687 of human variant 1 ErbB2 cDNA (NM-004448) and the transgene was inserted between HindIII and XbaI within the multiple cloning site(12). pNeu contains codons 1-692 of the rat neu oncogene cDNA (X03362). pE2neu contains codons 1-390 of NM-004448, a GAATTCGCT bridge, then codons 395-692 of X03362 (12, 13, 21). prmE2TM contains codons 1-687 of Rhesus (Macaca mulata) variant. X1 ErbB2 (XM_001090430)(GenScript) and was inserted between the NheI and XbaI sites in pVAX1. ph(es)E2TM is pE2TM with 5 codon substitutions (M198V, Q398R, F425L, H473R and A622T) and was inserted utilizing the Nhei and XbaI sites. Candidate DNA constructs were validated by transient transfection into NIH 3T3 cells using LipofectAMINE (Invitrogen, Carlsbad CA), following the manufacturer's instructions. Monoclonal antibodies TA-1 (Ab5; Calbiochem, San Diego, CA), N12, N29 (see Bacus S S, et al., 1992, Cancer Res. 52: 2580-9; and Stancovski I, et al., 1991, Proc Natl Acad Sci USA, 88: 8691-5; both hybridomas provided by Dr. Yosef Yarden, Weissman Inst) and trastuzumab (see Cho H S, et al., 2003, Nature, 421: 756-60. doi: 10.1038/nature01392) (Genentech) are used to characterize HER-2 epitopes. mAb 7.16.4 (Ab4, Calbiochem, San Diego, CA) was used to detect rat Neu epitope (Heeney J L, et al., Science, 2006, 313:462-6). PE-goat-anti-mouse IgG or PE-mouse-anti-human IgG were the secondary antibodies. Samples are analyzed on a BD FACScanto II and with FlowJo software (TreeStar, Ashland OR).

### Cell Lines and Reagents.

All tissue culture reagents were purchased from Invitrogen. Cell lines were cultured as described in detail in Jacob J B, et al., 2010, Cancer Res., 70: 119-28. doi: 10.1158/0008-5472.CAN-09-2554 and antigen-presenting cells (APC) 3T3/KB and 3T3/NKB generated as described in detail in Wei W Z, et al., 2005, Cancer Res. 65: 8471-8. doi: 10.1158/0008-5472.CAN-05-0934. Briefly, BALB/c NIH 3T3 fibroblasts were transfected with K^{d} and B7.1 (CD80) to generate 3T3/KB, or with the addition of HER2 for 3T3/EKB. 3T3/NKB similarly generated to express Neu was used for measuring anti-Neu Ab levels in the immune serum. The expression of the transgenes is validated by flow cytometry using mAb to Kd (SF1-1.1, Biolegend), B7.1 (CD80,), HER2 (TA-1/Ab5, Calbiochem) and Neu (Ab4). C57BL/6 lung epithelial cell line TC-1 expressing Kb and B7.1 was a gift from Dr. T. C. Wu (The Johns Hopkins University, Baltimore, MD). TC-1/E2 cells were transfected with wt HER-2 as previously described (Radkevich-Brown O. et al., (2010). Cancer Immunol Immunother. 59: 409-17. doi: 10.1007/s00262-009-0760-. TC-1 and TC-1/E2 cells are validated by tumor growth in C57BL/6 mice and by their expression of Kb as detected by mAb Af6-88.5.5.3 (eBioscience). Stable clones were maintained in G418 and puromycin medium (3T3/KB) or zeocin (3T3/NKB). SKOV3 cells were purchased from the American Type Culture Collection. D2F2 is a mouse mammary tumor that arose in a BALB/c hyperplastic alveolar nodule line, D2 described in Piechocki M P, et al., 2001, J. Immunol., 167: 3367-74; Medina D, et al., 1970, J. Natl. Cancer Inst., 45: 353-63; and Wei W Z, et al., 1986, Cancer Res. 46: 2680-5. D2F2 cells were co-transfected with pRSV/neo and pCMV/Neu, which encodes wild-type rat Neu to establish D2F2/Neu, as described in Jacob J, et al., 2006, Cell Immunol., 240: 96-106. doi: 10.1016/j.cellimm.2006.07.002. D2F2/E2 cells were generated by co-transfection with a HinDIII WAP-HER-2 expression cassette (6.9-kb) and linearized pRSV/neo as detailed in Piechocki M P, et al., 2001, J. Immunol., 167: 3367-74. D2F2/E2t cells were selected from D2F2/E2 cells by serial passage in BALB/c mice. D2F2 cells and derivatives are validated by tumor growth in BALB/c mice and by their expression of Kd as detected by mAb SF1-1.1. Expression of HER2 in D2F2/E2 and D2F2/E2t is verified by mAb Ab5, using flow cytometry. SK-BR-3 and SKOV3 cells were purchased from the American Type Culture Collection. Authentication of SKBR-3 and SKOV3 cells by short tandem repeat (STR) profiling was carried out with Promega's Cell ID System. Transfected cells were maintained in medium with 0.8 mg/mL G418 (Geneticin; Invitrogen) puromycin or zeocin.

### Derivation of D2F2/E2t

D2F2/E2 cells (2×10⁵ cells) were inoculated into the mammary fat pads of female BALB/c mice and the outgrowth was serially transplanted into naïve female BALB/c mammary fat pads for a total of 7 times. HER2 expression was evaluated after each passage by flow cytometry. Tumor cells were dissociated after the 7th transplantation and cloned. The cell D2F2/E2 clone cells maintaining with the highest HER2 expression were selected and designated D2F2/E2t. D2F2/E2t is maintained in medium containing 0.6 mg/ml G418.

### Validation of Cell Lines

Validation of cell lines by short tandem repeat (STR) profiling was carried out with Promega's Cell ID System as described by the supplier. BALB/c origin of D2F2 and derivatives was validated by tumor growth in BALB/c mice.

### DNA Electrovaccination

pcDNA/Neu encoding the extracellular and transmembrane domains of rat Neu was described in detail in Rovero S, et al., J. Immunol., 2000, 165:5133-42. pEF-Bos/granulocyte macrophage colony-stimulating factor (pGM-CSF) encoding murine GM-CSF was provided by Dr. N. Nishisaka at Osaka University, Osaka, Japan. Mice were electrovaccinated as described in detail in Wei W Z, et al., Int. J. Cancer, 1999, 81:748-54. Mice are anesthetized and 50 micrograms of test DNA construct admixed with 20 micrograms pGM-CSF in 50 μL PBS is injected intramuscularly (i.m.) in the quadriceps. Immediately following injection, square wave electroporation is applied over the injection site as described in Wei W Z, et al., Int. J. Cancer, 1999, 81:748-54 using a BTX830 (BTX Harvard Apparatus) or NEPA21 super electroporator (Nepa Gene) as described in detail in Roque-Afonso A M, et al., Antivir. Ther., 2007, 12:1255-63. The pulses at 100V with 20 msec duration are delivered 8 times in two opposite orientations. Mice were electrovaccinated one to three times at 2 wk intervals as described in the Results.

### Regulatory T Cell Depletion

In B6 HER-2 Tg mice, regulatory T cells (Treg) were depleted 10 days prior to vaccination by injecting i.p. 0.25 mg anti-CD25 mAb PC61.

### Immune Monitoring

Sera, peripheral blood lymphocytes (PBL) and(or) splenocytes (SC) were collected 2 wks following the last electrovaccination. Anti-HER-2 antibodies were measured by binding to HER-2 overexpressing SKOV3 cells using flow cytometry and antibody concentrations were calculated by regression analysis using mAb TA-1 as the standard as described in detail in Piechocki M P, et al, 2002, J. Immunol. Methods, 259: 33-42. Normal mouse serum or isotype matched mAb was the control. Anti-Neu antibodies were measured with 3T3/NKB cells and the standard curve is generated using mAb 7.16.4 (Ab4). Differences in antibody concentration are analyzed by the Student's t test.

ELISpot reagents were purchased from BD Biosciences. HER-2 reactive T cells were enumerated by IFN-γ ELISpot assay as described in detail in Jacob J B, et al., Cancer Res., 2010, 70:119-28; and Radkevich-Brown O, et al., Cancer Immunol. Immunother., 2010, 59:409-17. Peripheral blood (PBL) or spleen cells (SC) were incubated for 48 hours with the antigen presenting cells (APC) 3T3/EKB or TC-1/E2 (3T3/KB and TC-1 were negative controls) at an APC:lymphocyte ratio of 1:10 or recombinant HER2 or Neu protein (ecd-Fc fusion; SinoBiological). The results were expressed as number of cytokine-producing cells per 10⁶ SC. Data are analyzed using Student's t-test.

### BLOSUM Scores

To quantify the biochemical alteration from amino acid substitutions in cancer vaccines, the Blocks Substitutions Matrix (BLOSUM) score, see Henikoff S, et al., 1992, Proc Natl Acad Sci USA., 89: 10915-9; and Pertsemlidis A, et al., 2001, Genome Biol., 2: REVIEWS2002, was utilized.

Blocks Substitutions Matrix (BLOSUM) scores (described in detail in Styczynski M P, et al., 2008, Nat. Biotechnol., 26: 274-5. doi: 10.1038/nbt0308-274) are log of odds calculated from the frequency of amino acid substitutions in closely related protein sequences. BLOSUM62 was established by comparing protein blocks containing >62% sequence identity, see Styczynski M P, et al., 2008, Nat. Biotechnol., 26: 274-5. doi: 10.1038/nbt0308-274.

Each of the 20 amino acids is assigned a log odds score of +4 to +11. Higher values indicate the invariant nature of those residues. For the 190 possible amino acid substitutions, each is assigned a BLOSUM score of +3 to −4, with 0 indicating a substitution with a “neutral” probability”, i.e., equal probability of the alternative or original amino acid residues occurring.

Positive BLOSUM scores indicate conservative substitution with little impact on the protein. Very negative scores such as −4 indicate extremely rare substitution, which have 10,000 fold greater chance than the score of 0 for protein alterations.

### Peptide Binding Profiling

Peptide binding analysis was performed with a microarray that displays a library of 168 HER2 ECD 15-mer peptides with 11-mer overlap (JPT Peptide Technologies, Germany). The peptides were covalently immobilized on glass slides (PepStar Peptide Microarrays, JPT). Immune serum samples diluted 1:200 were incubated on the array for 1 hr at 30° C. Bound Ab was visualized with a fluorescently labeled anti-mouse IgG.

After washing and drying, the slides were scanned at 635 nm to obtain fluorescence intensity profiles. The images were quantified to generate a mean pixel value for each peptide. A color-coded heat map was computed to show relative fluorescence intensities.

### Inhibition of Tumor Cell Proliferation

Inhibition of tumor cell proliferation was measured by incubating human breast cancer cell line SK-BR-3 with HER2 immune serum in flat bottom 96 well plates. Serum from mice receiving blank pVax was the negative control. Gefitinib was used as a positive control. Cell survival was measured by Alamar Blue assay after 48 hours of incubation. Statistical significance was determined by Student's t test.

### Inhibition of Tumor Growth In Vivo

BALB HER2Tg mice were electrovaccinated twice with pE2Neu or ph(es)E2TM, at 2 week intervals. At 2 weeks after the second vaccination, D2F2/E2t cells were implanted into the #4 mammary fat pad and tumor growth was monitored by palpation twice per week. The tumor size was measured with a caliper and calculated by XY²/2 where X represents the longer axis and Y is the short axis.

### Results

### Hybrid HER2 Vaccine Constructs

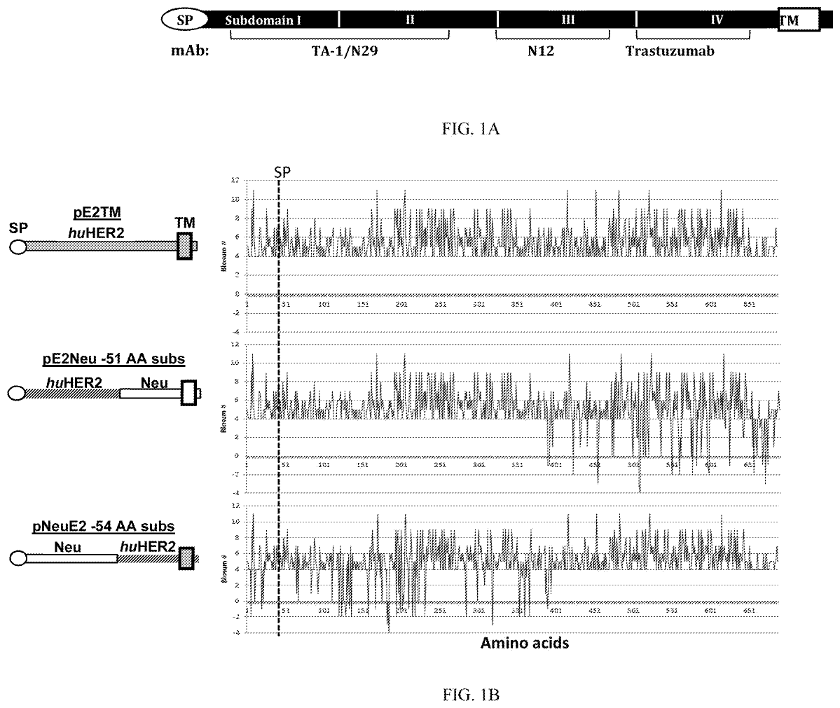

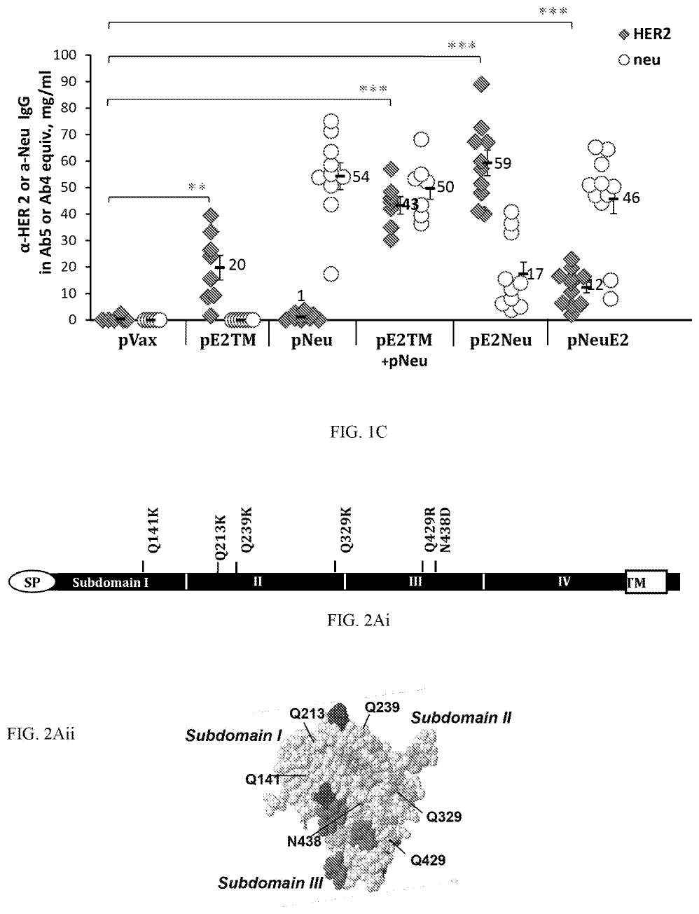

The amino acid BLOSUM scores were evaluated in three existing HER2 vaccines FIG. 1A/B): pE2TM encoding native HER2 ECD and TM domains and a 12 AA fragment of the ICD; and the hybrid constructs pE2Neu and pNeuE2, each containing distinct AA substitutions. FIG. 1B shows BLOSUM scores for the 687 residues in pE2TM (top row), ranging from +4 to +11. Relative to pE2TM, pE2Neu contains 51 substitutions in ECD subdomains III/IV and 3 extra residues (AEF) in ECD subdomain III, following codon #389, due to DNA cloning methodology. Thirty-two of the 51 substitutions were conservative, with BLOSUM scores=0. The other 19 scored −1 to −4. The reverse construct, pNeuE2, harbors 54 Neu-derived AA substitutions in subdomains I/II with 25 non-conservative changes scoring at −1 to −4.

Potency of these vaccines were compared in BALB/c (BALB) HER2 Tg mice (Styczynski M P, et al., 2008, Nat. Biotechnol., 26: 274-5. doi: 10.1038/nbt0308-274) after 3× electrovaccination at 2 wk intervals as described herein and antibody levels were measured 2 weeks following each vaccination. Vaccination controls were pVax, pNeu encoding rat Neu, and admixed pE2TM and pNeu. FIG. 1C shows HER2 binding Ab (shaded diamonds) induced by DNA electrovaccination. Compared to pE2TM, pE2Neu and admixed pE2TM+pNeu induced 2-3 fold elevation of IgG (59±15 or 43±9 vs 20±13 μg/ml), implicating heterologous Neu epitopes in promoting HER2 self-reactivity (FIG. 1C). The reverse hybrid, pNeuE2, did not enhance HER2 Ab even though anti-Neu Ab level was high (46±18 μg/ml), see Jacob J B, et al., 2010, Cancer Res., 70: 119-28. doi: 10.1158/0008-5472.CAN-09-2554.

HER2 Vaccines with Single Amino Acid Substitutions

The increased antibody response in pE2Neu vaccinated HER2 Tg mice indicates pE2Neu as an effective vaccine. However, the structural, biochemical and immunological impact of substituting 51 of 687 residues and inserting 3 additional residues (AEF) could not be readily elucidated, making it difficult to extrapolate to other TAA. A rational HER2 vaccine design with minimal amino acid substitutions was sought. A panel of six pE2TM vaccines harboring single amino acid substitutions were generated and tested.

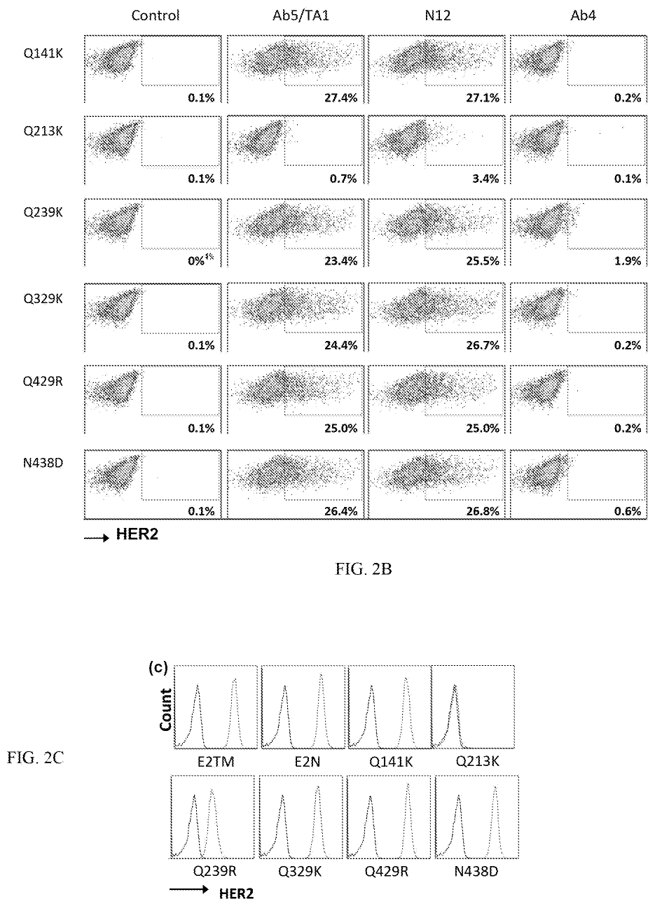

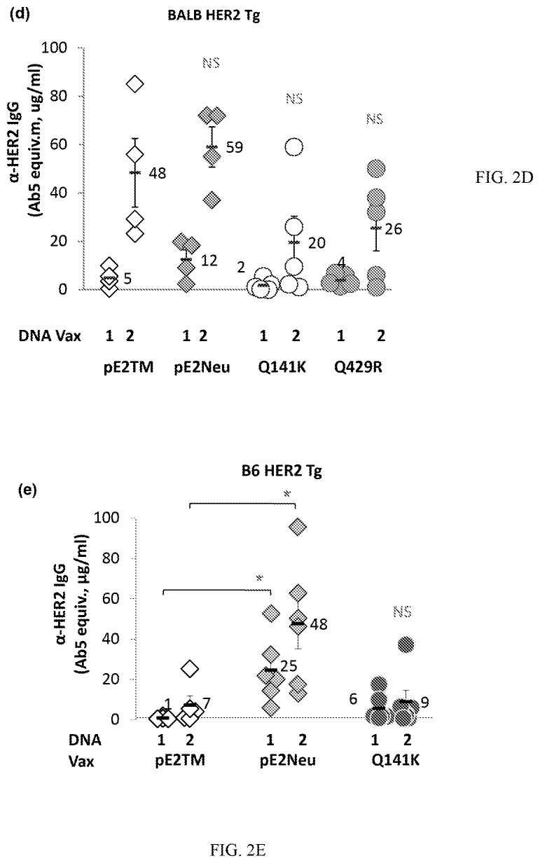

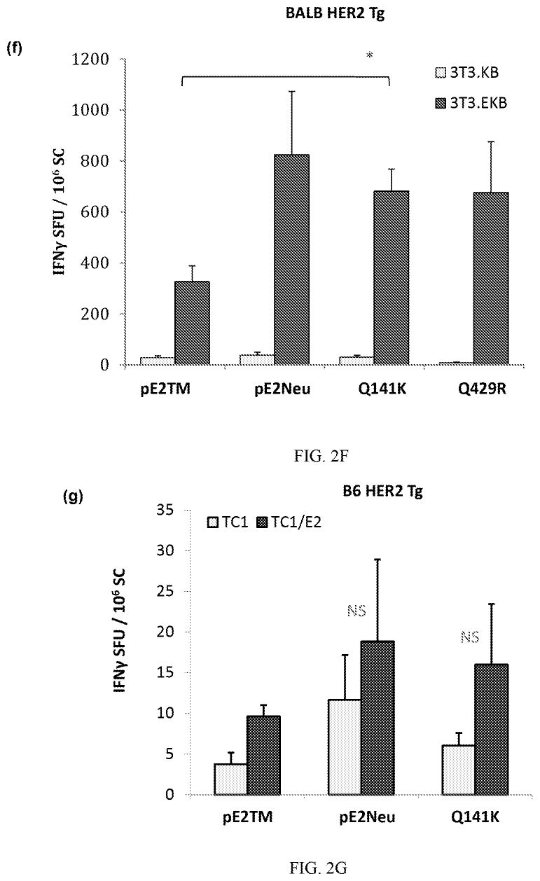

Glutamine (Q) or asparagine (N) located on the external surface of HER2 were replaced with AA carrying BLOSUM scores ≥1: Q141K, Q213K, Q239K, and Q329K, and Q429R and N438D (FIG. 2A). Stable expression of all mutants except Q231K was detected in transiently transfected 3T3 cells by anti-HER2 mAb TA-1 and N12, indicating preservation of the protein structure (FIG. 2B). Anti-neu mAb Ab4 was a negative control. To test immunogenicity in vivo, wt BALB/c mice were electrovaccinated once with the test constructs and HER2 binding Ab were measured two weeks following vaccination. All but Q213K test vaccines induced Ab (FIG. 2C).

Preliminary testing of the 5 stable constructs shows Q141K and Q429R mutants producing more consistent IgG response in BALB HER2 Tg mice. The immunogenicity of these two mutant HER2 vaccines were tested in then comparisoned with pE2TM and pE2Neu vaccines. BALB HER2 Tg mice electrovaccinated 2× produced 48±12, 59±8, 20±11 and 26±10 μg/ml HER2 binding Ab after vaccination with pE2TM, pE2Neu, pE2TM-Q141K and pE2TM-Q429R mutant constructs, respectively, showing no advantage of single residue substitutions (FIG. 2D). 2× vaccination of Treg-depleted C57BL/6 (B6) HER2 Tg mice with pE2TM, pE2Neu or pE2TM-Q141K induced 7±5, 48±12 and 9±6 μg/ml HER2 binding Ab (FIG. 2E). Treg were depleted 10 days before vaccination because this mouse strain responds poorly in the presence of Treg (see Radkevich-Brown O. et al., (2009) Cancer Res. 69: 212-8. doi: 10.1158/0008-5472.CAN-08-3092). Q141K mutants induced elevated HER2-specific IFN-γ-producing T cells in BALB HER2 Tg mice when compared with pE2TM (FIG. 2F) but not in B6 HER2 Tg (FIG. 2G). Therefore, the overall trend that single residue substitution is less effective than pE2Neu is shown consistent in both BALB HER2 Tg and C57BL/6 HER2 Tg mice.

Evolution-Selected HER2 Vaccine ph(es)E2TM

To produce HER2 proteins that closely resemble human HER2 in order to preserve most antigenic epitopes, yet with sufficient alteration to overcome HER2 self-tolerance, common amino acid substitutions were sought in HER2 sequences from twelve primate species that share >95% sequence identity with human HER2 (Table 1).

TABLE 1

| | | | | | | SEQ |
|---|---|---|---|---|---|---|
| | Sequence | | | | % | ID |
| Sequence | source | Start | End | Match | Match | NO: |
| huE2ectm | NP_004439 | 1 | 687 | | | 1 |
| protein | | | | | | |
| Bonobo- | XP_008955053 | 1 | 687 | 686 | 99 | 3 |
| XP_008955 | | | | | | |
| BolivSqMonkey- | XP_010328997 | 38 | 701 | 644 | 93 | 4 |
| XP | | | | | | |
| Chimp- | XP_003315512 | 1 | 687 | 685 | 99 | 5 |
| XP_0033155 | | | | | | |
| GoldSnNo- | XP_010377602 | 1 | 687 | 673 | 97 | 6 |
| Monkey-X | | | | | | |
| Gorilla- | XP_004041868 | 1 | 687 | 684 | 99 | 7 |
| XP_00404 | | | | | | |
| GrnMonkey- | XP_008011036 | 1 | 687 | 679 | 98 | 8 |
| XP_008 | | | | | | |
| OliveBaboon- | XP_003912981 | 1 | 687 | 681 | 99 | 9 |
| XP_0 | | | | | | |
| RhesusErbB2- | XM_001090430 | 1 | 687 | 677 | 98 | 10 |
| XP_0 | | | | | | |
| Rhesus- | U.S. Pat. No. | 1 | 687 | 677 | 98 | 11 |
| HER2prot2 | 7,282,365 | | | | | |
| SumOrangutan- | XP_009250137 | 1 | 658 | 630 | 91 | 12 |
| XP_ | | | | | | |
| WhChGibbon- | XP_003278275 | 37 | 699 | 654 | 95 | 13 |
| XP_00 | | | | | | |
| WhTufEar- | XP_002806904 | 1 | 687 | 660 | 96 | 14 |
| Marmoset | | | | | | |
| Macaca- | XP_005584091 | 98 | 784 | 677 | 98 | 15 |
| XP_005584 | | | | | | |

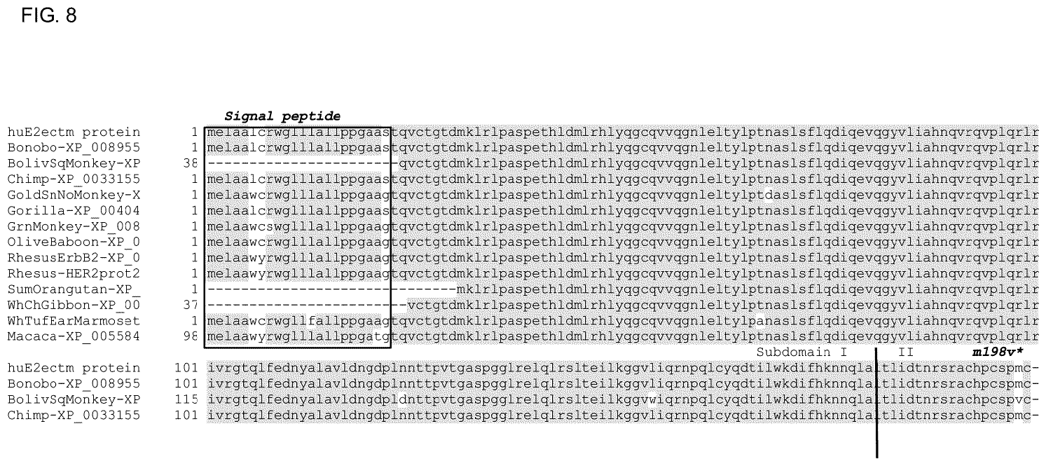

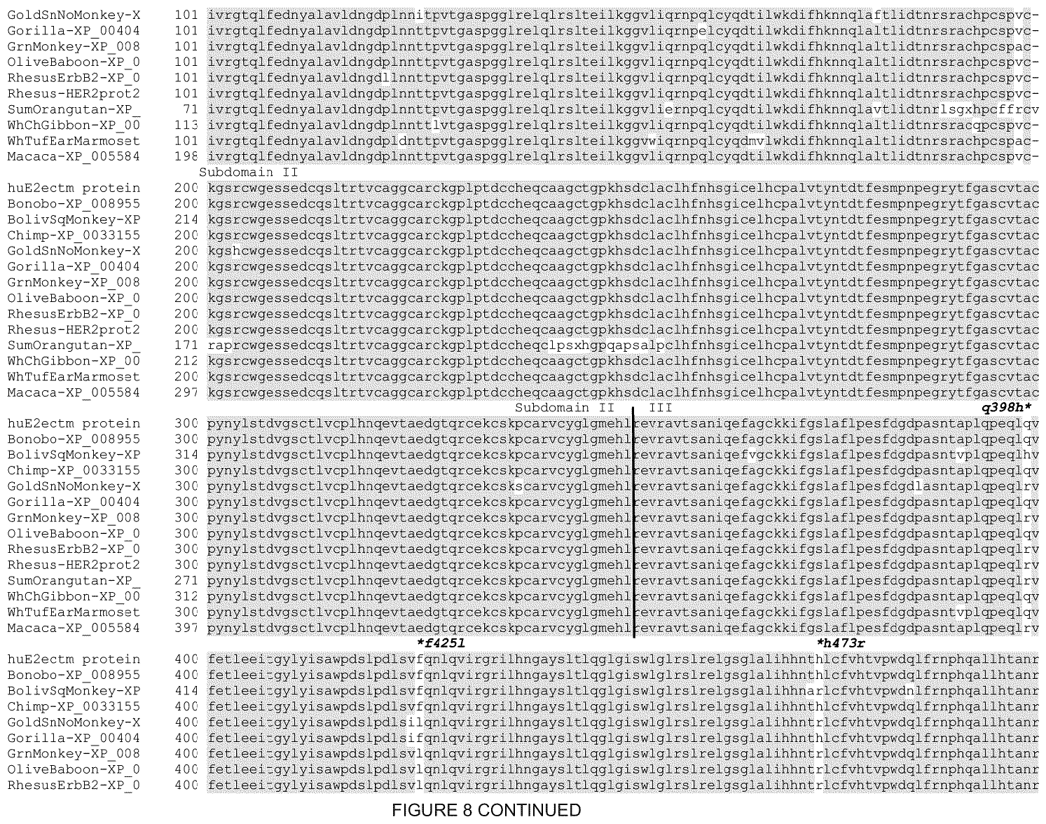

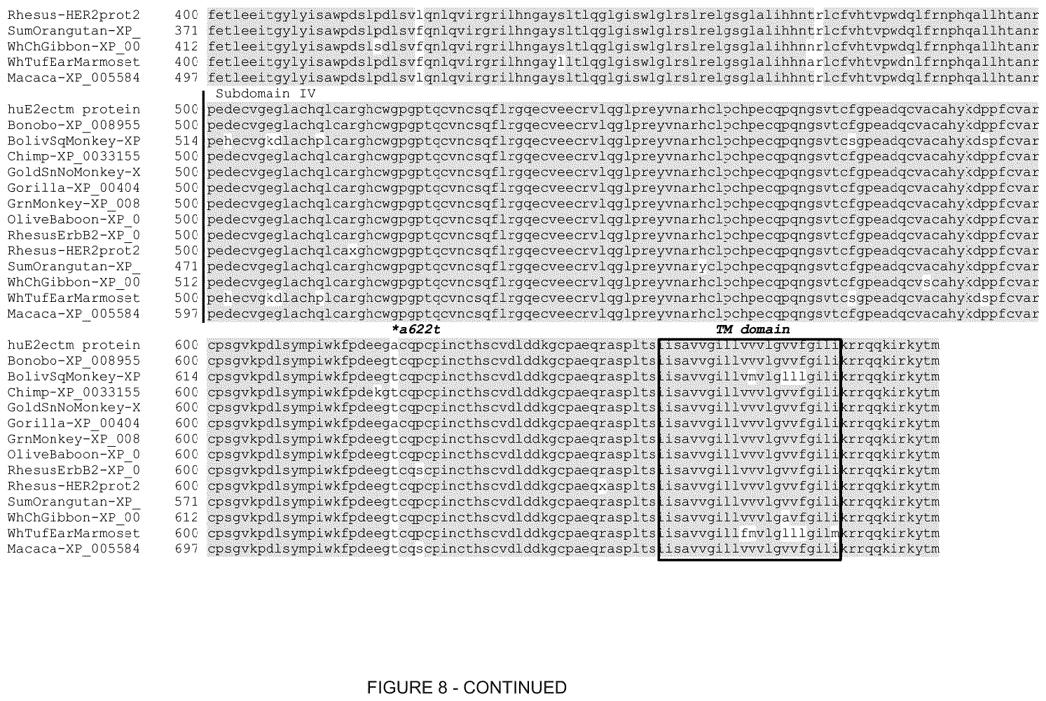

FIG. 8 shows the alignment of 14 protein sequences listed in Table 1. For this alignment, global protein alignment against reference molecule was used, wherein the reference molecule as huE2ectm protein, Region 1 to 687. Fourteen sequences were aligned and the total length of aligned sequences with gaps was 688 amino acids. Parameters: Scoring matrix: BLOSUM 62.

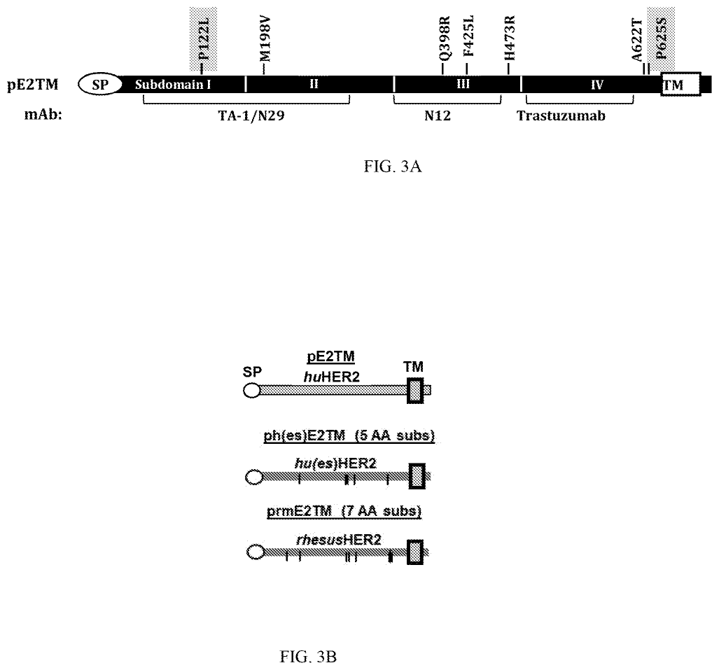

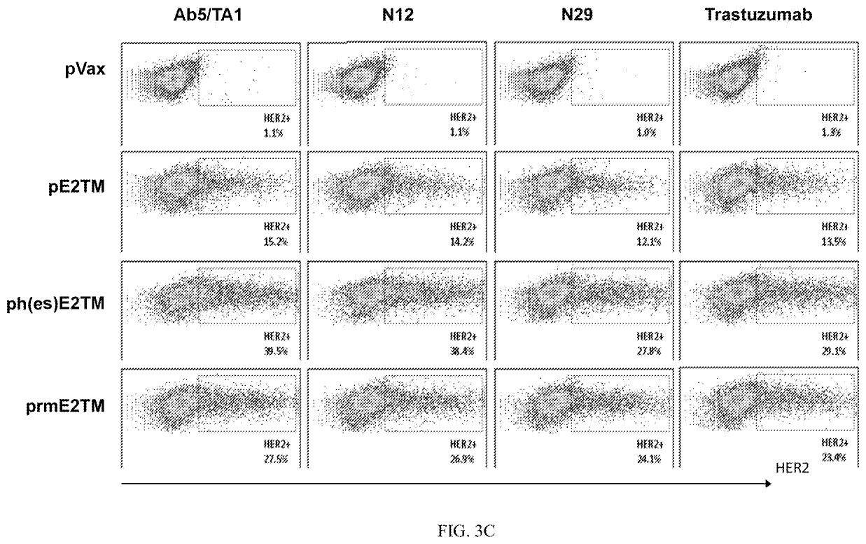

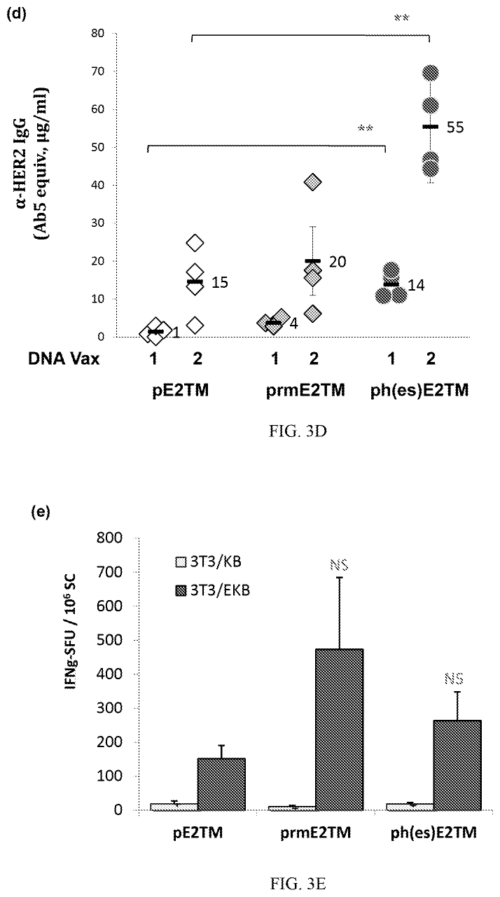

From this panel of ‘evolution-selected alterations’, 5 amino acids in HER2 ECD were found to be frequently replaced by a common residue (FIG. 8): M198V (ECD subdomain I), Q398R (III), F425L (III), H473R (III) and A622T (IV), see FIG. 3A. BLOSUM scores of these substitutions are all 0 and +1, indicating they are relatively neutral changes. A new HER2 vaccine, ph(es)E2TM, was generated to incorporate these five common substitutions as shown in FIG. 3B.

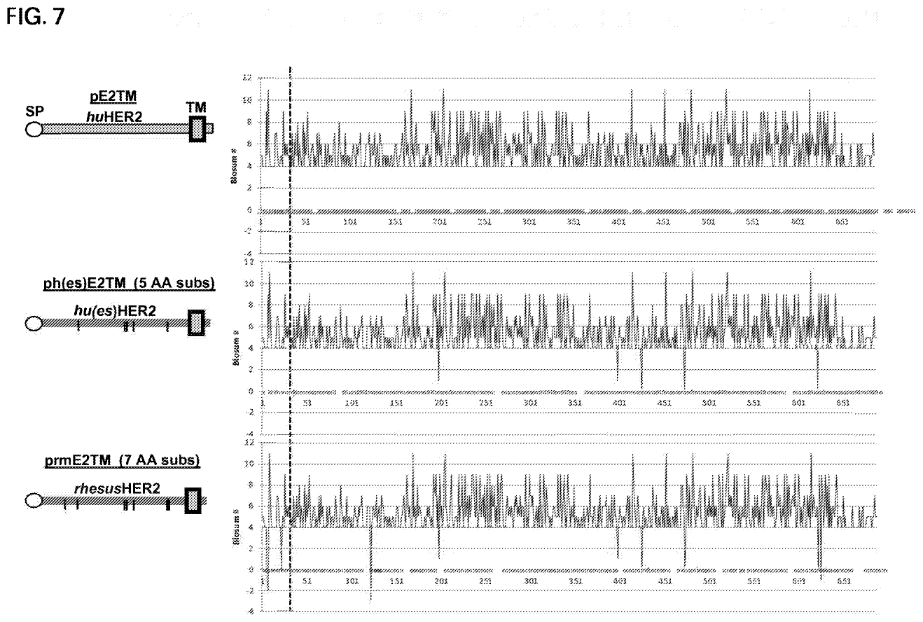

Alterations in ph(es)E2TM are readily quantifiable (FIG. 7). A rhesus monkey HER2 (rmE2TM) (XM_001090430) encodes 2 additional amino acid substitutions that are more drastic and not commonly observed: P122L in subdomain I (BLOSUM score=−3) and P625S in subdomain IV (score=−2) (shaded blocks) (Fattori E, et al., 2009, Hum. Gene Ther., 20: 253-65. doi: 10.1089/hum.2008.153)

To test whether drastic amino acid substitutions would detract immune response from native HER2, the prmE2TM DNA construct with all 7 residue substitutions was generated for comparison.

Expression and Immunogenicity of ph(es)E2TM and prmE2TM

Recombinant protein was measured by flow cytometry using 3T3 cells transiently transfected with ph(es)E2TM or prmE2TM (FIG. 3C). The controls were pVax and pE2TM. Anti-HER2 mAb TA1, N29, N12, and trastuzumab recognized both ph(es)E2TM and rmE2TM, recombinant proteins, indicating preservation of these epitopes.

Immunogenicity of ph(es)E2TM and prmE2TM versus native pE2TM was compared. BALB HER2 Tg mice were electrovaccinated twice and anti-HER2 response monitored (FIG. 3D-E). HER2 binding Ab increased by more than three fold in ph(es)E2TM immunized mice (55±6 vs 15±5, p<0.01), showing augmented HER2 immunogenicity from the 5 substitutions. In contrast, prmE2TM did not enhance HER2 Ab response (20±7 vs 15±6 μg/ml)(FIG. 3D). It is possible that P122L and P625S substitutions resulted in neoantigen to detract immunity from native HER2 (FIG. 3D). HER2-specific IFN-γ-producing T cells were induced in pE2TM, prmE2TM and ph(es)E2TM vaccinated mice at 152±39, 474±211 and 264±85 spot-forming units/10⁶ SC, respectively (FIG. 3E). There may be a trend toward higher T cell response in prmE2TM and ph(Es)E2TM immunized mice, but the difference was not statistically significant.

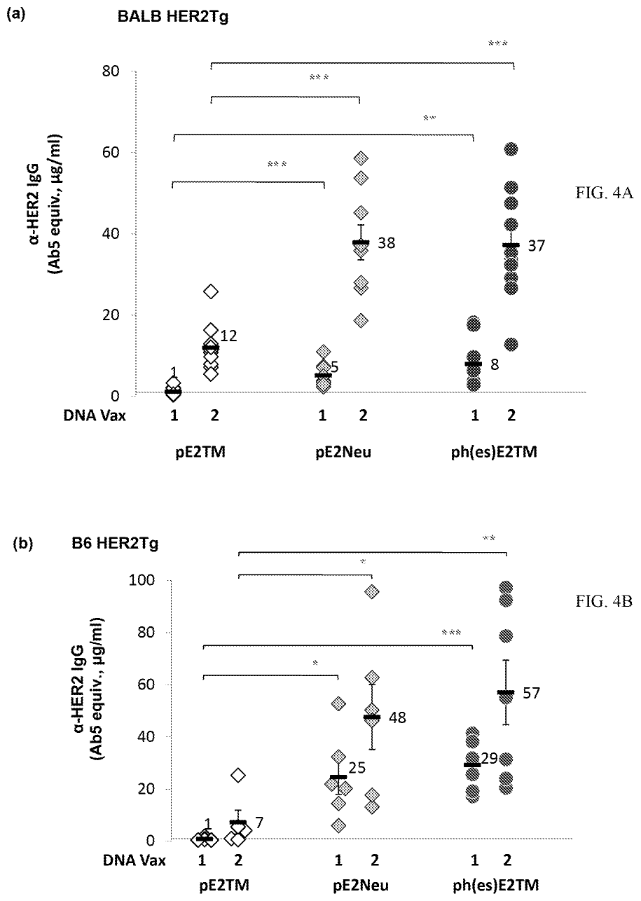

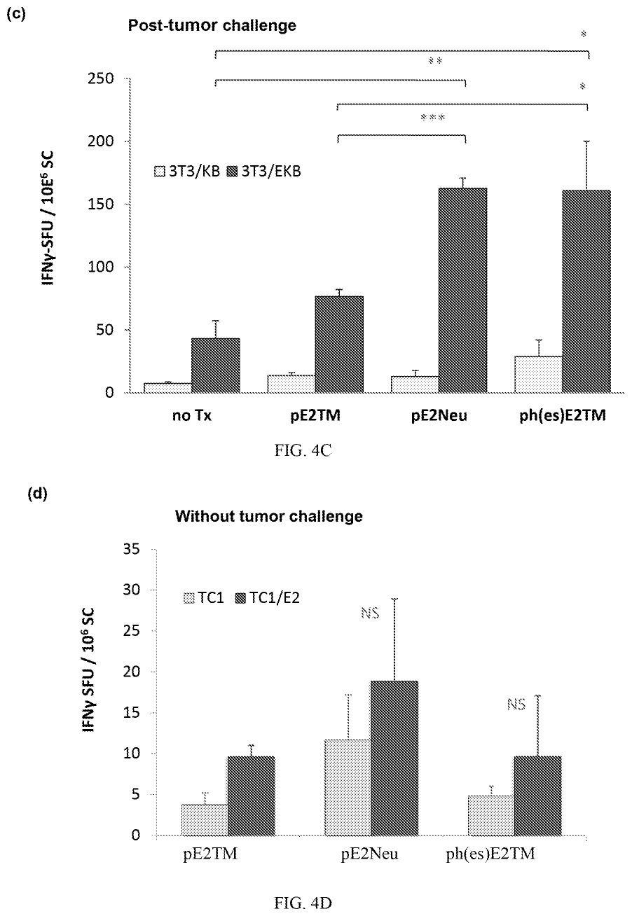

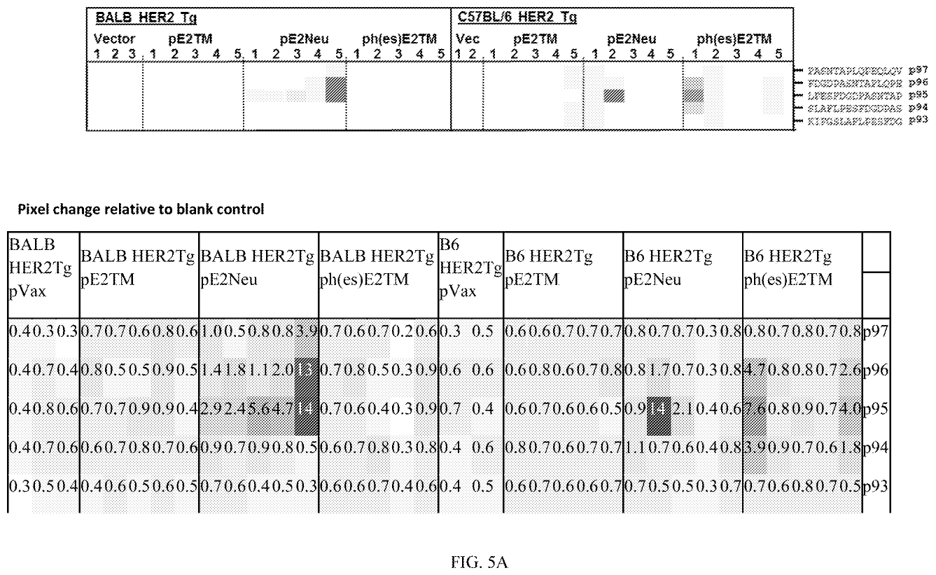

Immunogenicity of ph(es)E2TM was also compared with pE2Neu in BALB and B6 HER2 Tg mice. Mice were electrovaccinated twice, 2 weeks apart, and serum was collected two weeks after each vaccination. B6 HER2 Tg mice received mAb PC61 before vaccination to remove Treg. FIG. 4A shows pE2TM, pE2Neu and ph(es)E2TM inducing 12±1.8, 38±4.3 and 37±4.3 μg/ml HER2-binding Ab, respectively, in BALB HER2 Tg mice. ph(es)E2TM and pE2Neu induced comparable levels of HER2 binding IgG even though ph(es)E2TM has only 5 substituted residues. In Treg depleted, twice vaccinated B6 HER2 Tg mice, pE2Neu and ph(es)E2TM induced 48±12.4 μ/ml and 57±12.4 μg/ml HER2-binding Ab, respectively, compared to 7±4.6 μg/ml by pE2TM (FIG. 4B). Therefore, in both BALB and B6 HER2 Tg mice, ph(es)E2TM was as effective as pE2Neu in generating Ab to self HER2. Of the 5 substituted amino acids, four are located in ECD subdomains III and IV. M198V substitution in subdomain I has a BLOSUM score of +1. Incorporation of conservative AA substitutions selected and preserved during evolution resulted in an improved HER2 vaccine for antibody induction.

To measure HER2-specific T cell response, twice-vaccinated BALB HER2 Tg mice received intra-fat pad injection of syngeneic D2F2/E2t mammary tumor cells expressing human HER2 at 2 weeks post-2^{nd} vaccination. Immune SC harvested 3 weeks later showed a significant increase in T cell response in ph(es)E2TM or pE2Neu vaccinated mice, when compared to pE2TM immunization (FIG. 4C). A modest T cell response was induced in B6 HER2 Tg mice regardless of the vaccine. B6 HER2 Tg mice were not challenged with tumors (FIG. 4D). Taken together, ph(es)E2TM, like pE2Neu, induced elevated humoral immunity in both strains of mice and elevated T cell immunity in BALB HER2 Tg mice.

### Peptide Binding Profiles of Immune Serum

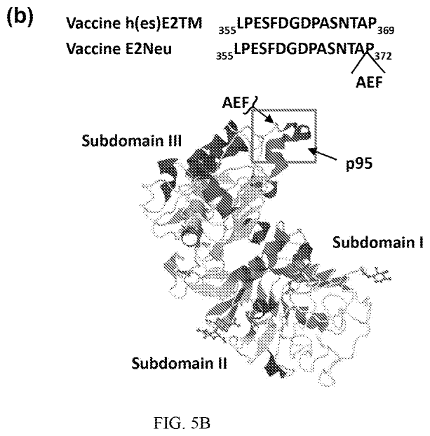

To determine the epitopes recognized by the immune serum, a library of 168 human HER2 15-mer peptides with 11-mer overlaps was used to evaluate the Ab binding profile. Peptides were covalently immobilized to glass slides. Immune serum was incubated on the peptide microarray slide at 1:200 dilution and bound antibodies were detected with a fluorescence labeled anti-mouse IgG and scanned at 635 nm. Specific binding was expressed by the fold increase in mean pixel value for a particular peptide over the average pixel values of all peptides excluding 3 non-specific binding peptides (85, 121 and 128) (FIG. 5A). An arbitrary cut-off of 2 fold increase was used to identify positive binding peptide.

A single P95 355LPESFDGDPASNTAP369 (SEQ ID NO:40) emerged as the target of the immune serum from B6 HER2 Tg mice that received pE2Neu (2/5) or ph(es)E2TM (2/5), but not pE2TM (0/5). In BALB HER2 Tg mice, pE2Neu immune serum recognized p95 (5/5), but not ph(es)E2TM or pE2TM immune serum. p95 in subdomain III is situated on the external surface of HER2 ECD (FIG. 5B). In pE2Neu, the 3 amino acid insertion (AEF) introduced during pE2Neu construction is located between residue 368-369 within p95, and may contribute to the exposure of p95 in E2Neu.

### Vaccine Induced Anti-Tumor Immunity

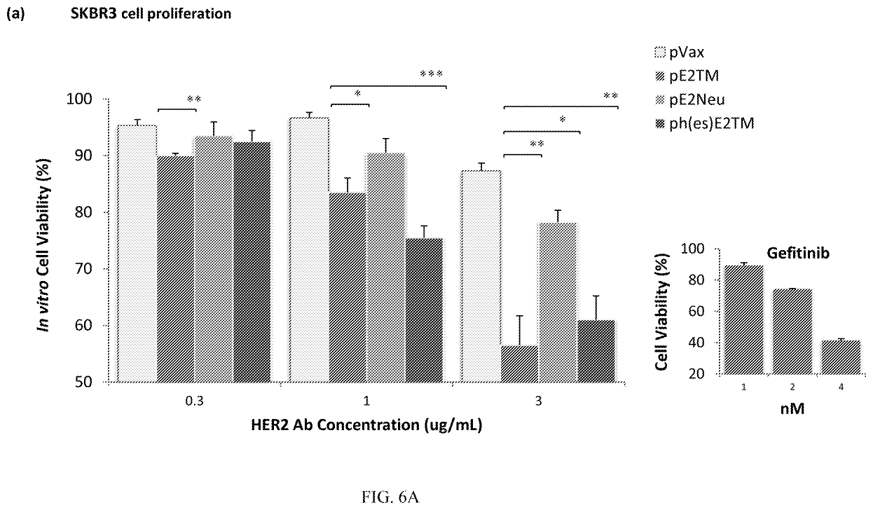

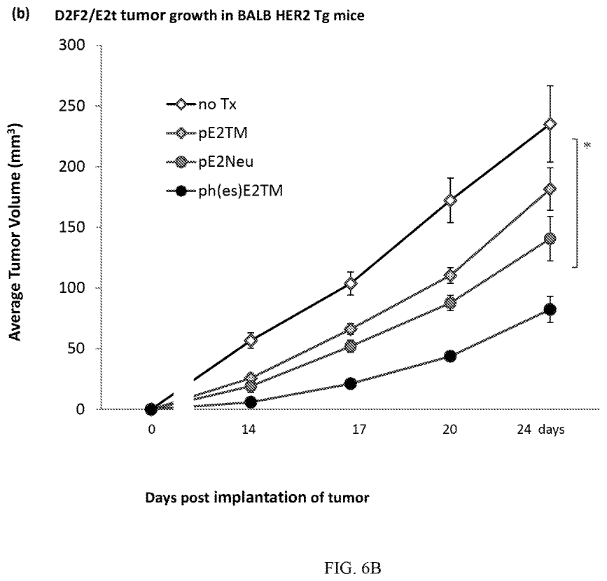

Functionality of HER2 immune serum was measured by incubating graded concentrations of immune serum from BALB HER2 Tg mice (FIG. 4A-B) with human SK-BR-3 cells for 48 hours (FIG. 6). Antibody concentrations were calculated based on their HER2 binding activity as determined by flow cytometry. Surviving cells were quantified by Alamar Blue. Reduction in cell survival at 45, 20 and 40%, was observed when the cells were incubated with 3 μg/ml of pE2TM, pE2Neu and ph(es)E2TM immune serum, respectively. At 1 μg/ml, only pE2TM (15% inhibition) and ph(es)E2TM (25% inhibition) immune sera inhibited tumor cell proliferation. Examination of IgG subtypes indicate similar IgG1/IgG2 composition in the three test sera.

The efficacy of controlling tumor growth in vivo was compared in BALB HER2 Tg mice (FIG. 6B). After two electrovaccinations, mice received 2×10⁵ BALB/c D2F2/E2t cells in their #4 fat pads. Tumor growth was monitored twice weekly. Tumor volume was reduced in mice whether they were vaccinated with pE2Neu or ph(es)E2TM, but reduction was greater in ph(es)E2TM immunized mice.

An effective HER2 cancer vaccine, ph(es)E2TM, was produced by substituting just 5 AA that occur frequently in closely related primates. These five substitutions (M198V, Q398R, F425L, H473R and A622T) are relatively conservative as defined by their BLOSUM scores of 0 to +1. This selection process resulted in a natural design template for generating tumor-associated self-antigens (TAA) vaccines to boost endogenous immunity. Vaccination with ph(es)E2TM induced HER2 immunity that inhibited tumor growth in HER2 Tg mice. Introduction of 2 additional drastic substitutions, P122L (score=−3) and P625S (score=−2), abolished elevated response to HER2, showing that inclusion of uncommon substitutions can be detrimental. Based on these findings with ph(es)E2TM, incorporation of evolution-selected, conservative substitutions may be most appropriate for boosting endogenous immunity to unmodified TAA.

The test vaccines were delivered by intramuscular DNA electroporation. Naked plasmid DNA can be readily generated and modified. It is stable and relatively easy to produce in large quantity. Intramuscular Delivery by i.m. injection is safe and consistent. Application of electroporation at the injection site enhances DNA uptake and expression with little adverse effect.

The importance of preserving wt HER2 ECD subdomains I and II in a vaccine construct was indicated by the poor vaccine response from pNeuE2, in which subdomains I and II were replaced with rat sequences. Critical epitopes may be lost or foreign epitopes created that detract from self HER2. Note that the predicted Nglycosylation sites in pNeuE2 ECD subdomain I differ significantly from wt pE2TM, ph(es)E2TM, or pE2neu. The post-translational glycosylation patterns may also contribute to altered antigenicity.

The ph(es)E2TM recombinant protein is recognized by a panel of four anti-HER2 mAbs that recognize specific epitopes in subdomains I/II, III and IV, showing structural preservation of HER2 ECD. Recombinant rmE2TM was also recognized by these mAbs, but prmE2TM was ineffective at elevating HER2 binding antibodies.

Epitope scanning revealed a linear epitope p95 355LPESFDGDPASNTAP369 (SEQ ID NO:40) preferentially recognized by pE2Neu immune serum from both strains and by ph(es)E2TM immune serum from B6 HER2 Tg mice. Treg depletion in B6 HER2 Tg mice prior to vaccination may result in a broader immune recognition. Overlapping peptides p94, p96 and p97 are also recognized by the reactive immune sera, revealing ESFDGDPASNT (SEQ ID NO:45) as the core peptide. The insertion of AEF between residues 368 and 369 at the C-terminus of p95 may expose p95 for B cell and Ab recognition. ph(es)E2TM does not contain this insertion and the closest substitution is Q398R.

ph(es)E2TM was tested in HER2 Tg mice of BALB/c and C57BL/6 backgrounds and provided elevated immune response in both strains. Although BALB/c and C57BL/6 mice have distinct MHC genotypes, and HER2 Tg mice of these two backgrounds showed very different intrinsic response to HER2 vaccines, ph(es)E2TM induced a stronger HER2 immunity compared to native pE2TM or pE2Neu in either strain. ph(es)E2TM with evolution-selected conservative residue substitutions represents a new and novel principle for vaccine formulation.

| Sequences | |
|---|---|
| SEQ ID NO: 1 - Homo sapiens (Human; “E2ectm”) NP_004439.2 (1-687) | |
| 1 | melaalcrwg lllallppga astqvctgtd mklrlpaspe thldmlrhly qgcqvvqgnl |
| 61 | eltylptnas lsflqdiqev qgvvliahnq vrqvplqrlr ivrgtqlfed nvalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytm |
| SEQ ID NO: 2 - h(es)E2ectm (human) (1-687; with substitutions M198V, | |
| Q398R, F4251, H473R, A622T) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcvglgmehl revravtsan |
| 361 | igefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 3 - Pan paniscus (Bonobo) XP_0089590.53.1 (1-145, 170-711) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO:4 - Saimiri boliviensis boliviensis (Bolivian Squirrel Monkey) | |
| XP_010328997.1 (38-701) | |
| 38 | qvctgtdmkl rlpaspethl dmlrhlvqgc qvvqgnlelt ylptnaslsf lqdiqevqgy |
| 98 | vliahnqvrq vplqrlrivr gtqlfednya lavldngdpl dnttpvtgas pgglrelqlr |
| 158 | slteilkggv wiqrnpqlcy qdtilwkdif hknnqlaltl idtnrsrach pcspvckgsr |
| 218 | cwgessedcq sltrtvcagg carckgplpt dccheqcaag ctgpkhsdcl aclhfnhsgi |
| 278 | celhcpalvt yntdtfesmp npegrytfga scvtacpvny lstdvgsctl vcplhnqevt |
| 338 | aedgtqrcek cskpcarvcy glgmehlrev ravtsaniqe fvgckkifgs laflpesfdg |
| 398 | dpasntvplq peqlhvfetl eeitgylyis awpdslpdls vfqnlqvirg rilhngaysl |
| 458 | tlqglaiswl glrslrelgs glalihhnar lcfvhtvpwd nlfrnphqal lhtanrpehe |
| 518 | cvgkdlachp lcarghcwgp gptqcvncsq flrgqecvee crvlqglpre yvnarhclpc |
| 578 | hpecqpqngs vtcsgpeadq cvacahykds pfcvarcpsg vkpdisympi wkfpdeegtc |
| 638 | qpcpincths cvdlddkgcp aeqrasplts iisavvgill vmvlglllgi likrrqqkir |
| 698 | kytm |
| SEQ ID NO: 5 - Pan troglodytes (Chimp) XP_003315512.2 (1-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglamehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdek gtcqpcpinc thscvdlddk gcpaeqrasp ltsiisavvg |
| 661 | illvvvlgvv fgilikrrqq kirkytm |
| SEQ ID NO: 6 - Rhinopithecus roxellana (Gold Snooted No Monkey | |
| XP_010377602.1 (1-687) | |
| 1 | melaawcrwg lllallppga agtqvctgtd mklrlpaspe thldmlrhly qgcqvvqgnl |
| 61 | eltylptdas lsflqdiqev qgyvliahnq vrqvplqrlr ivrgtqlfed nyalavldng |
| 121 | dplnnitpvt gaspgglrel qlrslteilk ggvliqrnpq lcvqdtilwk difhknnqla |
| 181 | ftlidtnrsr achpcspvck gshcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskscar vcyglgmehl revravtsan |
| 361 | iqefagckki fgslaflpes fdgdlasnta plqpeqlrvf etleeitgyl yisawpdslp |
| 421 | dlsilqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 7 - Gorilla gorilla gorilla (Gorilla) XP_004041868.1 (1-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpe lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefaackki fgslaflpes fdgdpasnta plqpeqlqvf etleeitgyl yisawpdslp |
| 421 | dlsifqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytm |
| SEQ ID NO: 8 - Chlorocebus sabaeus (Green Monkey) XP_008011036.1 (1-687) | |
| 1 | melaawcswg lilallppga agtqvctgtd mklrlpaspe thldmlrhly qgcqvvqgnl |
| 61 | eltylptnas lsflqdiqev qgyvliahnq vrqvplqrlr ivrgtqlfed nvalavldng |
| 121 | dplnnttpvt gaspgglrel qlrslteilk ggvliqrnpq lcyqdtilwk difhknnqla |
| 181 | ltlidtnrsr achpcspack gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | vnylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgeala chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 9 - Papio anubis (Olive Baboon) XP_003912981.1 (1-687) | |
| 1 | melaawcrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynvlstdvgs ctlvcplhnq evtaedgtqr cekcskpcar vcyglgmehl revravtsan |
| 361 | iqefaackki fgslaflpes fdgdpasnta plqpeqlqvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 10 - Macaca mulatta (Rhesus) XP_001090430.1 (1-687) | |
| 1 | melaawyrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dllnnttpvt gaspgglrel qlrslteilk ggvliqrnpq lcyqdtilwk difhknnqla |
| 181 | ltlidtnrsr achpcspvck gsrcwgesse dcqsltrtvc aagcarckgp yptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqscpinc thscvdlddk gcpaeqrasp ltsiisavvg |
| 661 | illvvvlgvv fgilikrrqq kirkytm |
| SEQ ID NO: 11 - Macaca mulatta (Rhesus-HER2prot2) US7282365 B2 (#41) | |
| (1-687) | |
| 1 | melaawyrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcvqdtilwk difhknnqla |
| 181 | ltlidtnrsr achpcspvck gsrcwgesse dcqsitrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcaxghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqxasp ltsiisavvg |
| 661 | illvvvlgvv fgilikrrqq kirkytm |
| SEQ ID NO: 12 - Pongo abelii (Sumatran Orangutan) XP_009250137.1 | |
| (1-221, 229-666) | |
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliernpq lcyqdtilwk difhknnqla vtlidtnrls gxhpcffrcv raprcwgess |
| 181 | edcqsltrtv caggcarckg plptdccheq clpsxhgpqa psalpclhfn hsgicelhcp |
| 241 | alvtyntdtf esmpnpegry tfgascvtac pynylstdvg sctlvcplhn qevtaedgtq |
| 301 | rcekcskpca rvcyglgmeh lrevravtsa niqefagckk ifgslaflpe sfdgdpasnt |
| 361 | aplqpeqlrv fetleeitgy lyisawpdsl pdlsvfqnlq virgrilhng aysltlqglg |
| 421 | iswlglrslr elgsglalih hntrlcfvht vpwdqlfrnp hqallhtanr pedecvgegl |
| 481 | achqlcargh cwgpgptqcv ncsqflrgqe cveecrvlqg lpreyvnary clpchpecqp |
| 541 | qngsvtcfgp eadqcvacah vkdppfcvar cpsgvkpdls ympiwkfpde egtcqpcpin |
| 601 | cthscvdldd kgcpaeqras pltsiisavv gillvvvlgv vfgilikrrq qkirkytm |
| SEQ ID NO: 13 - Nomascus leucogenys (WhChGibbon) XP_003278275.1 (37-699) | |
| 37 | vctgtdmklr lpaspethld mlrhlyqgcq vvqgnlelty lptnaslsfl qdiqevqgyv |
| 97 | liahnqvrqv plqrlrivrg tqlfednyal avldngdpln nttlvtgasp gglrelqlrs |
| 157 | iteilkggvl iqrnpqlcyq dtilwkdifh knnqlaltli dtnrsracqp cspvckgsrc |
| 217 | wgessedcqs ltrtvcaggc arckgpiptd ccheqcaagc tapkhsdcla clhfnhsgic |
| 277 | elhcpalvty ntdtfesmpn pegrytfgas cvtacpynyl stdvgsctlv cplhnqevta |
| 337 | edgtqrcekc skpcarvcyg lgmehlrevr avtsaniqef agckkifgsl aflpesfdgd |
| 397 | pasntaplqp eqlqvfetle eitgylyisa wpdslsdlsv fqnlqvirgr ilhngayslt |
| 457 | iqglgiswlg lrslrelgsg lalihhnnrl cfvhtvpwdq ifrnphqall htanrpedec |
| 517 | vaeglachql carghcwgpg ptqcvncsqf lrgqecveec rvlqglprey vnarhclpch |
| 577 | pecqpqngsv tcfgpeadqc vscahykdpp fcvarcpsgv kpdlsvmpiw kfpdeegtcq |
| 637 | pcpincthsc vdlddkgcpa eqraspltsi isavvgillv vvlgavfgil ikrrqqkirk |
| 697 | ytm |
| SEQ ID NO: 14 - Callithrix jacchus (WhTufEarMarmoset) XP_002806904.1 | |
| (1-687) | |
| 1 | melaawcrwg llfallppga agtqvctatd mklrlpaspe thldmlrhly qgcqvvqgnl |
| 61 | eltylpanas lsflqdiqev qgyvliahnq vrqvplqrlr ivrgtqlfed nyalavldng |
| 121 | dpldnttpvt gaspgglrel qlrslteilk ggvwiqrnpq lcyqdmvlwk difhknnqla |
| 181 | ltlidtnrsr achpcspack gsrcwgesse dcqsltrtvc aggcarckgp lptdccheqc |
| 241 | aagctgpkhs dclacihfnh sgicelhcpa lvtyntdtfe smpnpegryt fgascvtacp |
| 301 | ynylstdvgs ctlvcplhnq evtaedgtqr cekcskpcar vcyglamehl revravtsan |
| 361 | iqefagckki fgslaflpes fdgdpasntv plqpeqlqvf etleeitgyl yisawpdslp |
| 421 | dlsvfqnlqv irgrilhnga ylltlqglgi swlglrslre lgsglalihh narlcfvhtv |
| 481 | pwdnlfrnph qallhtanrp ehecvgkdla chplcarghc wgpgptqcvn csqflrgqec |
| 541 | veecrvlqgl preyvnarhc lpchpecqpq ngsvtcsgpe adqcvacahy kdspfcvarc |
| 601 | psgvkpdlsv mpiwkfpdee gtcqpcpinc thscvdlddk gcpaeqrasp ltsiisavvg |
| 661 | illfmvlgll lgilmkrrqq kirkytm |
| SEQ ID NO: 15 - Macaca fascicularis (Macaca) XP_005584091.2 (98-784) | |
| 98 | melaawyrwg lllallppga tgtqvctgtd mklrlpaspe thldmlrhly qgcqvvqgnl |
| 158 | eltylptnas lsflqdiqev qgyvliahnq vrqvplqrlr ivrgtqlfed nyalavldng |
| 218 | dplnnttpvt gaspgglrel qlrslteilk ggvliqrnpq lcvqdtilwk difhknnqla |
| 278 | ltlidtnrsr achpcspvck gsrcwgesse dcqsltrtvc aggcarckgp lptdccheqc |
| 338 | aagctgpkhs dclaclhfnh sgicelhcpa lvtyntdtfe smpnpegryt fgascvtacp |
| 398 | vnylstdvgs ctlvcplhnq evtaedgtqr cekcskpcar vcyglgmehl revravtsan |
| 458 | igefagckki fgslaflpes fdgdpasnta plqpeqlrvf etleeitgyl yisawpdslp |
| 518 | dlsvlqnlqv irgrilhnga ysltlqglgi swlglrslre lgsglalihh ntrlcfvhtv |
| 578 | pwdqlfrnph qallhtanrp edecvgegla chqlcarghc wgpgptqcvn csqflrgqec |
| 638 | veecrvlqgl preyvnarhc lpchpecqpq ngsvtcfgpe adqcvacahy kdppfcvarc |
| 698 | psgvkpdlsv mpiwkfpdee gtcqscpinc thscvdlddk gcpaeqrasp ltsiisavvg |
| 758 | illvvvlgvv fgilikrrqq kirkytm |
| SEQ ID NO: 16 - Human ERBB2ectm-delta 16 NP_004439.2 (1-632, 649-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq icyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegrvt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thspltsiis avvgillvvv lgvvfgilik |
| 661 | rrqqkirkyt m |
| SEQ ID NO: 17 - Human (es)E2ectm-delta 16 (1-632, 649-687; with | |
| substitutions M198V, Q398R, F425L, H473R, A622T) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcvglamehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thspltsiis avvgillvvv lgvvfgilik |
| 661 | rrqqkirkyt m |
| SEQ ID NO: 18 - Feline E2ectm (wt) Translate of JN990983 (1-687) | |
| 1 | melaawcrwg lllallpsga tgtqvctgtd mklrlpaspe thldmlrhly qgcqvvqgnl |
| 61 | eltylhanas lsflqdiqev qgyvliahnq vkqvplqrlr ivrgtqlfed nyalavldng |
| 121 | dpldsgtpat gaalgglrel qlrslteilk ggvliqrnpq lchqdtilwk difhknnqla |
| 131 | lmlidtnrsr acqpcspack dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlrvf ealeeitgyl yisawpdslp |
| 421 | nlsvfqnlrv irgrvlhdga ysltlqglgi swlglrslre lgsglalihr nsrlcfvhtv |
| 481 | pwdqlfrnph qallhsanrp edecagegla cvplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gtcqpcpinc thscadldek gcpaeqrasp vtsiiaavvg |
| 661 | illvvvvglv lgilikrrrq kirkytm |
| SEQ ID NO: 19 - Feline (es)E2ectm (1-687; with substitutions A198V, | |
| R398Q, F425L, R473H, T622A) | |
| 1 | melaawcrwg lllallpsga tgtqvctgtd mklrlpaspe thldmlrhly qgcqvvqgnl |
| 61 | eltylhanas lsflqdiqev qgyvliahnq vkqvplqrlr ivrgtqlfed nyalavldng |
| 121 | dpldsgtpat gaalgglrel qlrslteilk ggvliqrnpq lchqdtilwk difhknnqla |
| 181 | lmlidtnrsr acqpcspvck dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlqvf ealeeitgyl yisawpdslp |
| 421 | nlsvlqnlrv irgrvlhdga ysltlqglgi swlglrslre lgsglalihr nshlcfvhtv |
| 481 | pwdqlfrnph qallhsanrp edecagegla cyplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gacqpcpinc thscadldek gcpaeqrasp vtsiiaavvg |
| 661 | illvvvvglv lgilikrrrq kirkytm |
| SEQ ID NO: 20 - Feline (es)E2ectm-delta16 Feline (es)E2ectm (1-632, | |
| 649-687) | |
| 1 | melaawcrwg lllallpsga tgtqvctgtd mklrlpaspe thldmlrhly qgcqvvqgnl |
| 61 | eltylhanas lsflqdiqev qgvvliahnq vkqvplqrlr ivrgtqlfed nvalavldng |
| 121 | dpldsgtpat gaalgglrel qlrslteilk ggvliqrnpq lchqdtilwk difhknnqla |
| 181 | lmlidtnrsr acqpcspvck dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlqvf ealeeitgyl yisawpdslp |
| 421 | nlsvlqnlrv irgrvlhdga ysltlqglgi swlglrslre lgsglalihr nshlcfvhtv |
| 431 | pwdqlfrnph qallhsanrp edecagegla cyplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gacqpcpinc thspvtsiia avvgillvvv vglvlgilik |
| 661 | rrrqkirkvt m |
| SEQ ID NO: 21 - Dog E2ectm(wt) NP_001003217 (1-687) | |
| 1 | melaawcrwg lllallpsga agtavctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dpleggipap gaapgglrel qlrslteilk ggvliqrspq lchqdtilwk dvfhknnqla |
| 131 | ltlidtnrsr acppcspack dahcwgassg dcqsltrtvc aggcarckgp qptdccheqc |
| 241 | aagctgpkhs dclaclhfnh sgicelhcpa lvtyntdtfe smpnpegryt fgascvtscp |
| 301 | ynylstdvgs ctlvcplnnq evtaedgtqr cekcskpcar vcvglgmehl revravtsan |
| 361 | igefagckki fgslaflpes fegdpasnta plqpeqlrvf ealeeitgyl yisawpdslp |
| 421 | nlsvfqnlrv irgrvlhdga ysltlqglgi swlglrslre lgsglalihr narlcfvhtv |
| 431 | pwdqlfrnph qallhsanrp eeecvgegla cyplcahghc wgpgptqcvn csqflrgqec |
| 541 | veecrvlqgl preyvkdryc lpchsecqpq nasvtcfgse adqcvacahy kdppfcvarc |
| 601 | psgvkpdlsf mpiwkfadee gtcqpcpinc thscadldek gcpaearasp vtsiiaavvg |
| 661 | illavvvglv lgilikrrrq kirkvtm |
| SEQ ID NO: 22 - Dog (es)E2ectm NP_001003217 (1-687; with substitutions | |
| A198V, R398Q, F425L, R473H, T622A) | |
| 1 | melaawcrwg lllallpsga agtqvctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dplegaipap gaapgglrel qlrslteilk ggvliqrspq lchqdtilwk dvfhknnqla |
| 181 | ltlidtnrsr acppcspvck dahcwgassg dcqsltrtvc aggcarckgp qptdccheqc |
| 241 | aagctgpkhs dclaclhfnh sgicelhcpa lvtvntdtfe smpnpegryt fgascvtscp |
| 301 | ynylstdvgs ctlvcplnnq evtaedgtqr cekcskpcar vcyglgmehl revravtsan |
| 361 | iqefagckki fgslaflpes fegdpasnta plqpeqlqvf ealeeitgyl yisawpdslp |
| 421 | nlsvlqnlrv irgrvlhdga ysltlqglgi swlglrslre lgsglalihr nahlcfvhtv |
| 481 | pwdqlfrnph qallhsanrp eeecvgegla cyplcahghc wgpgptqcvn csqflrgqec |
| 541 | veecrvlqgl preyvkdryc lpchsecqpq ngsvtcfgse adqcvacahy kdppfcvarc |
| 601 | psgvkpdlsf mpiwkfadee gacqpcpinc thscadldek gcpaeqrasp vtsiiaavvg |
| 661 | illavvvglv lgilikrrrq kirkvtm |
| SEQ ID NO: 23 - Dog (es)E2ectm-delta16 Dog (es)E2ectm (1-632, 649-687) | |
| 1 | melaawcrwg lllallpsga agtqvctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dpleggipap gaapgglrel qlrslteilk ggvliqrspq lchqdtilwk dvfhknnqla |
| 181 | ltlidtnrsr acppcspvck dahcwgassg dcqsltrtvc aggcarckgp qptdccheqc |
| 241 | aagctgpkhs dclaclhfnh sgicelhcpa lvtyntdtfe smpnpegryt fgascvtscp |
| 301 | ynylstdvgs ctlvcpinnq evtaedgtqr cekcskpcar vcyglgmehl revravtsan |
| 361 | iqefagckki fgslaflpes fegdpasnta plqpeqlqvf ealeeitgvl yisawpdslp |
| 421 | nlsvlqnlrv irgrvlhdga ysltlqglgi swlglrslre lgsglalihr nahlcfvhtv |
| 481 | pwdqlfrnph qallhsanrp eeecvgegla cyplcahghc wgpgptqcvn csqflrgqec |
| 541 | veecrvlqgl pryvvkdryc lpchsecqpq ngsvtcfgse adqcvacahy kdppfcvarc |
| 601 | psgvkpdlsf mpiwkfadee gacqpcpinc thspvtsiia avvgillavv vglvlgilik |
| 661 | rrrqkirkyt m |
| ERBB2 cDNAs for human, cat, dog; wt, (es), (es)d16 | |
| Technical note: The feline ERBB2 cDNAs, for example, are derived from | |
| accession #JN990983 (which inventors cloned). This has a 135 nt 5′ UTR, | |
| where the orf begins at nt 136. The numbering of the feline ERBB2 cDNA | |
| derivatives is based on this. In the case of (es) sequences, the sub- | |
| stitutions are indicated. Finally, since the ectm vaccines include only the | |
| first ~687 codons, a stop codon was added, as indicated. | |
| SEQ ID NO: 24 - Homo sapiens (Human; “E2ectm”) NM_004448 (1-2061, +TGA) | |
| 1 | atggagctgg cggccttgtg ccgctggggg ctcctcctcg ccctcttgcc ccccggagcc |
| 61 | gcgaacaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | aacccgctaa acaataccac ccctgtcaca ggggcctccc caggaggcct gcgggagcta |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc gatgtgtaag |
| 601 | ggctcccgct gctggggaga gagttctgag gattgtcaga gcctgacgcg cactgtctgt |
| 661 | gccgatggct gtgcccgcta caagggacca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccatgccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | aaggtgacag cagagaatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatgggg acccagcctc caacactgcc ccgctccagc cagagcagct ccaagtgttt |
| 1201 | gagactctgg aagagatcac aggttaccta tacatctcag catggccgga cagcctgcct |
| 1261 | gacctcagcg tcttccagaa cctgcaagta atccggggac aaattctgca caatgacgcc |
| 1321 | tactcgctga ccctgcaagg gctgggcatc agctggctgg ggctgcgctc actgagggaa |
| 1381 | ctgggcagtg gactggccct catccaccat aacacccacc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcgcatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctgg tcgtggtctt gggggtagtc tttaggatcc tcatcaagcg acggcagcag |
| 2041 | aagatccgga agtacacgat gtga |
| SEQ ID NO: 25 -h(es)E2tm (human) | |
| 1 | atggagctgg cggccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | aaactcacct acctgcccac caatgccagc ctgtccttcc tgcaagatat ccaggaggta |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctactacc aagacacgat tttgtgaaag gacatcttcc acaagaacaa ccagctggct |
| 541 | ctcacactga tagacaccaa ccgctctcgg gcctgccacc cctgttctcc ggtgtgtaag |
| 601 | ggctcccgct gctggggaga gagttctgag gattgtcaga gcctgacgcg cactgtctgt |
| 661 | gccggtggct gtgcccgctg caaggggcca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgaa |
| 841 | tccatgccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | gaggtgacag cagaggatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggcta caagaaaatc tttgggagcc tggcatttct gccggagagc |
| 1141 | tttgatgggg acccagcctc caacactgcc ccgctccagc cagagcagct cagagtgttt |
| 1201 | gagactctgg aagagatcac aggttaccta tacatctcag catggccgga cagcctgcct |
| 1261 | gacctcagcg tcctccagaa cctgcaagta atccggggac gaattctgca caatggcgcc |
| 1321 | tactcgctga ccctgcaagg gctgggcatc agctggctgg ggctgcgctc actgagggaa |
| 1381 | ctgggcagtg gactggccct catccaccat aacacccgcc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagta tcagccccag aatagctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcacatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctag tcgtgatctt ggaggtggtc tttgggatcc tcatcaagcg acggcagcaa |
| 2041 | aagatccgga agtacacgat gtaa |
| SEQ ID NO:26 - Human E2ectm-delta 16 NM_004448 (1-1898, 1947-2061, +TAA) | |
| 1 | atggagctgg cggccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctcca ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc gatgtgtaag |
| 601 | ggctcccgct gctgggaaga gagttctgag gattgtcaga gcctgacgcg cactgtctgt |
| 661 | gccgatggct gtgcccgcta caaggggcca ctgcccacta actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccataccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | aaggtgacag cagagaatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatggga acccagcctc caacactgcc ccgctccagc cagagcagct ccaagtgttt |
| 1201 | gagactctgg aagagatcac aggttaccta tacatctcag catggccgga cagcctgcct |
| 1261 | gacctcagcg tcttccagaa cctgcaagta atccggggac gaattctgca caatggcgcc |
| 1321 | tactcgctga ccctgcaagg gctgggcatc agctggctgg ggctgcgctc actgagggaa |
| 1381 | ctgggcagtg gactggccct catccaccat aacacccacc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcgcatgcc aaccttgccc catcaactgc acccactccc ctctgacgtc catcatctct |
| 1921 | gcggtggttg gcattctgct ggtcgtggtc ttgggggtgg tctttgggat cctcatcaag |
| 1981 | cgacggcagc agaagatccg gaagtacacg atgtaa |
| SEQ ID NO: 27 - Human (es)E2ectm-delta 16 | |
| 1 | atggaactgg cgaccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc ggtgtgtaag |
| 601 | ggctcccgct gctggggaga gagttctgag gattgtcaga gcctgacgcg cactgtctgt |
| 661 | gccggtggct gtgcccgctg caaggggcca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccatgccca atcccgaggg ccggtataca ttcggcgcca gctgtgtgac tgcctgtccc |
| 901 | tacaactacc tttctacgga cgtaggatcc tgcaccctcg tctgccccct gcacaaccaa |
| 961 | gaggtgacag cagaggatgg aacacagcgg tgtgagaagt gcagcaagcc ctatgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatgggg acccagcctc caacactgcc ccgctccagc cagagcagct cagagtgttt |
| 1201 | gagactctgg aaaagatcac aggttaccta tacatctcag catggccgga cagcctgcct |
| 1261 | gacctcagcg tcctccagaa cctgcaaata atccggggac gaattctgca caatggcgcc |
| 1321 | tactcgctga ccctgcaagg gctgggcatc agctggctgg ggctgcgctc actgaggaaa |
| 1381 | ctgggcagtg gactggccct catccaccat aacacccgcc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaagggctc cccagggagt atgtgaatgc caagcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcacatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctgg tcgtggtctt gggggtggtc tttgggatcc tcatcaagcg acggcagcag |
| 2041 | aagatccgga agtacacgat gtaa |
| SEQ ID NO: 28 - Feline E2ectm (wt) JN990983 (136-2196, +TGA) | |
| 1 | atggagctgg cggcctggtg ccgctggggg ctcctcctcg ccctcctgcc ctccggagcc |
| 61 | acgggcaccc aagtgtacac cggcacagac atgaagctgc ggctcccagc cagtcccgag |
| 121 | acccacctgg acatgctccg ccacctctac cagggctgtc aagtgataca gggcaacctg |
| 181 | gagctcacct acctgcatgc caatgccagc ctctccttcc tgcaggatat ccaggaggtg |
| 241 | caaggctatg tgctcattgc ccacaaccaa gtgaaacagg tcccactgca gaggctacga |
| 301 | atcgtgcgag gcacccagct ctttgaggac aactacgccc tggccgtgct ggacaacgga |
| 361 | gacccactgg acagtggcac ccctgctaca ggggctgccc taggagggct gcgggagctg |
| 421 | cagctccgaa gcctcacaga gatcctgaag ggaggggtcc tcattcagcg gaacccgcag |
| 481 | ctctgccacc aggacacgat tctgtggaag gacatcttcc acaagaacaa ccagctggcc |
| 541 | ctcatgctga tagacaccaa ccgctctcgg gcctgccaac cctgttctcc agcttgtaaa |
| 601 | gactcccact gctggggagc aagttccggg gactgtcaga gcttgactcg aactgtctgt |
| 661 | gctggcggct gtgcccgctg caagggcccg cagcccaccg actgctgcca cgagcaatgt |
| 721 | gctgctggct gcacgggccc caagcattct gactgcctgg cctgcctcca cttcaaccac |
| 781 | agtggcatct gtgagctgca ctgcccagcc ctggtcacct acaacacgga caccttcgaa |
| 841 | tccatgccca accctgaggg ccgttatacc ttcggtgcca gctgtgtgac tgcctgtccc |
| 901 | tacaactacc tgtctacgga cgtgggatcc tgcaccctgg tctgtcccct gaacaaccaa |
| 961 | gaggtaacag ctaaggatgg aacacagcgg tgtgagaaat gcagcaagcc ctgtgcccga |
| 1021 | gtgtgctacg gcctaggcat ggagcacctg cgggaggcga gggcagtcac cagtgccaac |
| 1081 | atccaagaat ttgtcggctg caagaagatc tttgggagcc tggcgtttct gccagagagc |
| 1141 | tttgaggggg acccagcctc caacactgcc cccctgcagc ctgagcagct cagagtgttt |
| 1201 | gaggctctgg aggagattac aggttacctg tacatctcag cgtggccaga cagcttgcct |
| 1261 | aacctcagtg tcttccagaa cctcagagtg atccggggcc gagttctgca tgacggtgct |
| 1321 | tactcgctga cccttcaagg gctgggcatc agctggctgg ggctgcgctc gctgggggag |
| 1381 | ctggacagtg gactggccct catccaccgc aactcccgcc tctgcttcgt acacacggtg |
| 1441 | ccctgggacc agctcttccg gaacccccac caggccctgc tccacagcgc caaccggcca |
| 1501 | gaggacgagt gcgcgggtga gggcctggcc tgctatccgc tgtgtgccca cgggcactgc |
| 1561 | tggggtccgg gacccaccca gtgtgtcaac tgcagccagt tccttcgggg ccaggagtgc |
| 1621 | gtggaggaat gccgagtatt gcaggggctt ccccgggagt atgtgaagga taggttctgt |
| 1681 | ctgccatgcc acccgaagtg tcagccccag aatggctcag tgacctgctt gggctcggaa |
| 1741 | gctgaccagt gtgtggcctg tgcccactac aaggaccctc ctttctgtgt ggctcgctgc |
| 1801 | cccagtgggg tgaaacctga cctctccttc atgcccatct ggaagttcgc agatgaggag |
| 1861 | ggcacgtgcc agccatgccc catcaactgc acccactcct gtgcgaacct ggacgagaag |
| 1921 | ggctgccccg ccgagcagag agccagccct atgacgtcca tcattgctgc tgtggtgggc |
| 1981 | attctgctgg tcgtggttgt ggggctggtc cttggcatcc taatcaagcg aaggcggcag |
| 2041 | aagatccgga agtacacgat gtga |
| SEQ ID NO: 29 - Feline (es)ERBB2ectm JN990983 (136-2196; with substitutions | |
| c728t, t729g, a1327c, g1328a, a1329g, t1408c, g1553a, a1999g, g2001c; taa) | |
| 1 | gtacaagaat gaagttgtgg agctgagagt cccctgcgtc gtgccccgag agccgaacag |
| 61 | agctcccagg cagccgcccg gcccttcgca gcccggtcca gcccgagcca tggggccgga |
| 121 | gccgcagtga gcaccatgga gctggcggcc tggtgccgct gggggctcct cctcgccctc |
| 181 | ctgccctccg gagccacggg cacccaagtg tgcaccggca cagacatgaa gctgcggctc |
| 241 | ccagccagtc ccgagaccca cctggacatg ctccgccacc tctaccaggg ctgtcaagtg |
| 301 | gtacagggca acctggagct cacctacctg catgccaatg ccagcctctc cttcctgcag |
| 361 | gatatccagg aggtgcaagg ctatgtgctc attgcccaca accaagtgaa acaggtccca |
| 421 | ctgcagaggc tacgaatcgt gcgaggcacc cagctctttg aggacaacta cgccctggcc |
| 481 | gtgctggaca acggagaccc actggacagt ggcacccctg ctacaggggc tgccctagga |
| 541 | gggctgcggg agctgcagct ccgaagcctc acagagatcc tgaagggagg ggtcctcatt |
| 601 | cagcggaacc cgcagctctg ccaccaggac acgattctgt ggaaggacat cttccacaag |
| 661 | aacaaccagc tggccctcat gctgatagac accaaccgct ctcgggcctg ccaaccctgt |
| 721 | tctccagtgt gtaaagactc ccactgctgg ggagcaagtt ccggggactg tcagagcttg |
| 781 | actcgaactg tctgtgctgg cggctgtgcc cgctgcaagg gcccgcagcc caccgactgc |
| 841 | tgccacgagc aatgtgctgc tggctgcacg ggccccaagc attctgactg cctggcctgc |
| 901 | ctccacttca accacagtgg catctgtgag ctgcactgcc cagccctggt cacctacaac |
| 961 | acggacacct tcgaatccat gcccaaccct gagggccgtt ataccttcgg tgccagctgt |
| 1021 | gtgactgcct gtccctacaa ctacctgtct acggacgtgg gatcctgcac cctggtctgt |
| 1081 | cccctgaaca accaagaggt gacagctgag gatggaacac agcggtgtga gaaatgcagc |
| 1141 | aagccctgtg cccgagtgtg ctacggccta ggcatggagc acctgcggga ggcgagagca |
| 1201 | gtcaccagtg ccaacatcca agaatttgtc ggctgcaaga agatctttgg gagcctggcg |
| 1261 | tttctgccag agagctttga gggggaccca gcctccaaca ctgcccccct gcagcctgag |
| 1321 | cagctccagg tgtttgaggc tctggaggag attacaggtt acctgtacat ctcagcgtgg |
| 1381 | ccagacagct tgcctaacct cagtgtcctc cagaacctca gagtgatccg ggaccgagtt |
| 1441 | ctgcatgacg gtgcttactc gctaaccctt caagggctgg gcatcagctg gctggggctg |
| 1501 | cgctcgctgc gagagctggg cagtgggctg gccctcatcc accgcaactc ccacctctgc |
| 1561 | ttcgtacaca cggtgccctg ggaccagctc ttccggaacc cccaccaggc cctgctccac |
| 1621 | agcgccaacc ggccagagga cgagtgcgcg ggtgagggcc tggcctgcta tccgctgtgt |
| 1681 | gcccacgggc actgctgggg tccgggaccc acccagtgtg tcaactgcag ccagttcctt |
| 1741 | cggggccagg agtgcgtgga ggaatgccga gtattgcagg gacttccccg ggagtatgtg |
| 1801 | aaggataggt tctgtctgcc atgccacccg gagtgtcagc cccaaaatgg ctcagtgacc |
| 1861 | tgcttgggct cggaagctga ccagtgtgtg gcctgtgccc actacaagga ccctcctttc |
| 1921 | tgtgtggctc gctgccccag tggggtgaaa cctgacctct ccttcatgcc catctggaag |
| 1981 | ttcgcagatg aggaggacgc ctgccagcca tgccccatca actgcaccca ctcctgtgcg |
| 2041 | gacctggacg agaagggctg ccccgccgag cagagagcca gccctgtgac gtccatcatt |
| 2101 | gctgctgtgg tgggcattct gctggtcgtg gttgtggggc tggtccttgg catcctaatc |
| 2161 | aagcgaaggc ggcagaagat ccggaagtac acgatgtaa |
| SEQ ID NO: 30 - Feline (es)ERBB2ectm-delta16 Feline (es)ERBB2ectm | |
| (136-2033, 2082-2196; taa) | |
| 1 | gtacaagaat gaagttgtgg agctgagagt cccctgcgtc gtgccccgag agccgaacag |
| 61 | agctcccagg cagccgcccg gcccttcgca gcccggtcca gcccaagcca tggggccgga |
| 121 | gccgcagtaa gcaccatgga gctggcggcc tggtgccgct ggggactcct cctcgccctc |
| 181 | ctgccctccg gagccacggg cacccaagtg tgcaccggca cagacatgaa gctgcggctc |
| 241 | ccagccagtc ccgagaccca cctggacatg ctccgccacc tctaccaggg ctgtcaagtg |
| 301 | gtacagggca acctggagct cacctacctg catgccaatg ccagcctctc cttcctgcag |
| 361 | gatatccagg aagtgcaaga ctatgtgctc attgcccaca accaagtgaa acaggtccca |
| 421 | ctgcagaggc tacgaatcgt gcgaggcacc cagctctttg aggacaacta cgccctggcc |
| 481 | gtgctggaca acggagaccc actggacagt ggcacccctg ctacaggggc tgccctagga |
| 541 | gggctgcggg agctgcagct ccgaagcctc acagagatcc tgaagggagg ggtcctcatt |
| 601 | cagcggaacc cgcagctctg ccaccagaac acgattctgt gaaaggacat cttccacaag |
| 661 | aacaaccagc tggccctcat gctgatagac accaaccgct ctcgggcctg ccaaccctgt |
| 721 | tctccagtgt gtaaagactc ccactgctgg ggagcaagtt ccggggactg tcagagcttg |
| 781 | actcgaacta tctgtgctgg cggctgtgcc cgctgcaagg gcccgcagcc caccgactgc |
| 841 | taccacgagc aatgtgctgc tggctgcacg ggccccaagc attctagctg cctggcctgc |
| 901 | ctccacttca accacagtga catctgtgag ctgcactgcc cagccctggt cacctacaac |
| 961 | acggacacct tcgaatccat gcccaaccct gagggccgtt ataccttcgg tgccagctgt |
| 1021 | gtgactgcct gtccctacaa ctacctgtct acggacgtgg gatcctgcac cctggtctgt |
| 1081 | cccctaaaca accaagaggt gacagctgag gatggaacac agcggtgtga gaaatgcagc |
| 1141 | aagccctgtg cccgagtgtg ctacggccta ggcatggagc acctgcggga ggcgagggca |
| 1201 | gtcaccagtg ccaacatcca agaatttgtc ggctgcaaga agatctttgg gagcctggcg |
| 1261 | tttctgccag agagctttga gggggaccca gcctccaaca ctgcccccct gcagcctgag |
| 1321 | cagctccagg tgtttgaggc tctggaggag attacaggtt acctgtacat ctcagcgtgg |
| 1381 | ccagacagct tgcctaacct cagtgtcctc cagaacctca gagtgatccg gggccgagtt |
| 1441 | ctgcatgaca gtgcttactc gctgaccctt caagggctgg gcatcagctg gctggggctg |
| 1501 | cgctcgctgc gggagctggg cagtgggctg gccctcatcc accgcaactc ccacctctgc |
| 1561 | ttcgtacaca cggtgccctg ggaccagctc ttccggaacc cccaccaggc cctgctccac |
| 1621 | agcgccaacc ggccagagga cgagtgcgcg ggtgagggcc tggcctgcta tccgctgtgt |
| 1681 | gcccacgggc actgctgggg tccgggaccc acccagtgtg tcaactgcag ccagttcctt |
| 1741 | cggggccagg agtgcgtgga ggaatgccga gtattgcagg gacttccccg ggagtatgtg |
| 1801 | aaggataggt tctgtctgcc atgccacccg gagtgtcagc cccagaatgg ctcagtgacc |
| 1861 | tgcttgggct cggaagctga ccagtgtgtg gcctgtgccc actacaagga ccctcctttc |
| 1921 | tgtgtggctc gctgccccag tggggtgaaa cctgacctct ccttcatgcc catctggaag |
| 1981 | ttcgcagatg aggaggacgc ctgccagcca tgccccatca actgcaccca ctcccctgtg |
| 2041 | acgtccatca ttgctgctgt ggtgggcatt ctgctggtcg tggttgtggg gctggtcctt |
| 2101 | ggcatcctaa tcaagcgaag gcggcagaag atccggaagt acacgatgta a |
| SEQ ID NO: 31 - Canine E2ectm (wt) NM_001003217 (1-2058, +TGA) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aaatggtaca ggggaacctg |
| 181 | gagctcactt acctgcctgc caatgccagc ctgtccttcc tgcaggatat ccaggaggtg |
| 241 | cagggctatg tgctcattgc tcacagccaa atgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | caacttcgaa gcctcacaga gatcctgaag gaaggggtct tgattcagcg gagcccgcag |
| 481 | ctctgccacc aggacacgat tttatggaag gacgtcttcc ataagaacaa ccaactggcc |
| 541 | ctcacgctga tagacaccaa ccgcttttcg gcctacccgc cctgttctcc agcttgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgagaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttaatgggg acccagcctc caacactgcc cccctacagc ctgagcagct cagagtgttt |
| 1201 | gaggctctgg aggagatcac aggttacctg tacatctcag cgtggccaga cagcctgcct |
| 1261 | aacctcagtg tcttccagaa cctgcgagta atccggggac gagttctgca tgatggtgcc |
| 1321 | tactcgctga ccctgcaagg gctgggcatc agctgactgg ggctgcgctc gctgcggaaa |
| 1381 | ctgggcagtg ggctggccct catccaccgc aacgcccgcc tttgcttcgt gcacacggtg |
| 1441 | ccctgggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggaatgcgtg |
| 1621 | gaagaatgcc gagtactgca ggggctgccc cgagagtatg tgaaggacag gtactgtcta |
| 1681 | ccgtgccact cagagtgtca gccccagaat ggctcagtga cctgtttcgg atcggaagct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | acttgccagc cgtaccccat caactgcacc cactcctgtg cgaacctgga cgagaagggc |
| 1921 | tgtcccgccg agcagagagc cagccctgtg acatccatca ttaccgctgt ggtgggcatt |
| 1981 | ctgctggctg tggtcgtggg gctggtcctc ggcatcctga tcaagcgaag gcggcagaag |
| 2041 | atccggaagt acactatgtg a |
| SEQ ID NO: 32 - Canine (es)E2ectm NM _001003217 (1-2058, +TGA; with | |
| substitutions c593t, t594g, a1192c, g1193a, a1194g, t1273c, g1418a, | |
| a1861g, t1863c)) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aagtggtaca gggaaacctg |
| 181 | gagctcactt acctgcctgc caatgccagc ctgtccttcc tacaggatat ccaggaagtg |
| 241 | cagggctatg tgctcattgc tcacagccaa gtgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | cagcttcgaa gcctcacaga gatcctgaag ggaggggtct tgattcagcg gagcccgcag |
| 481 | ctctgccacc aggacacgat tttatggaag gacgtcttcc ataagaacaa ccagctggcc |
| 541 | ctcacgctga tagacaccaa ccgcttttcg gcctgcccgc cctgttctcc agtgtgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgaaaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttgatgggg acccagcctc caacactgcc cccctacagc ctgagcagct ccaggtgttt |
| 1201 | gaggctctgg aggagatcac aggttacctg tacatctcag cgtggccaga cagcctgcct |
| 1261 | aacctcagtg tcctccagaa cctgcgagta atccggggac gagttctgca tgatggtgcc |
| 1321 | tactcgctga ccctgcaagg gctgggcatc agctggctgg ggctgcgctc gctgcgggaa |
| 1381 | ctgggcagtg ggctggccct catccaccgc aacgcccacc tttgcttcgt gcacacggtg |
| 1441 | ccctgggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggagtgcgtg |
| 1621 | gaggaatgcc gagtactgca ggggctgccc cgagaatatg tgaaggacag gtactgtcta |
| 1681 | ccgtgccact cagagtatca gccccagaat ggctcagtga cctgtttcgg atcggaggct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | gcctgccagc cgtgccccat caactgcacc cactcctgtg cggacctgga cgaaaagggc |
| 1921 | tgtcccgccg agcagagagc cagccctgtg acatccatca ttgccgctgt ggtaggcatt |
| 1981 | ctgctggctg tggtcgtggg gctggtcctc ggcatcctga tcaagcgaag gcggcagaag |
| 2041 | atccggaagt acactatgtg a |
| SEQ ID NO: 33 - Dog (es)E2ectm-deltal6 Dog (es)E2ectm (with 1896-1943 | |
| deleted) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aagtggtaca ggggaacctg |
| 181 | gagctcactt acctgcctgc caatgccagc ctgtccttcc tgcaggatat ccaggaggtg |
| 241 | cagggctatg tgctcattgc tcacagccaa gtgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | cagcttcgaa gcctcacaga gatcctgaag ggaggggtct tgattcagcg gagcccgcag |
| 481 | ctctgccacc aggacacgat tttatggaag gacgtcttcc ataagaacaa ccagctggcc |
| 541 | ctcacgctga tagacaccaa ccgcttttcg gcctgcccgc cctgttctcc agtgtgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgagaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttgatgggg acccagcctc caacactgcc cccctacagc ctgagcagct ccaggtgttt |
| 1201 | gaggctctgg aggagatcac aggttacctg tacatctcag cgtggccaga cagcctgcct |
| 1261 | aacctcagtg tcctccagaa cctgcgagta atccggggac gagttctgca tgatggtgcc |
| 1321 | tactcgctga ccctgcaagg gctgggcatc agctggctgg ggctgcgctc gctgcgggaa |
| 1381 | ctgggcagtg ggctggccct catccaccgc aacgcccacc tttgcttcgt gcacacggtg |
| 1441 | ccctaggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggagtgcgtg |
| 1621 | gaggaatgcc gagtactgca ggggctgccc cgagagtatg tgaaggacag gtactgtcta |
| 1681 | ccgtgccact cagagtgtca gccccagaat ggctcagtga cctgtttcgg atcggaggct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | gcctgccagc cgtgccccat caactgcacc cactcccctg tgacatccat cattgccgct |
| 1921 | gtggtgggca ttctgctggc tgtggtcgtg gggctggtcc tcggcatcct gatcaagcga |
| 1981 | aggcggcaga agatccggaa gtacactatg tga |

PCR primers for construction of human ERBB2-delta16, feline ERBB2-delta16
and canine ERBB2-delta16 and derivatives thereof.
SEQ ID NO: 34 - huE2-D16R
5′-GGAGTGGGTGCAGTTGATGG-3′
SEQ ID NO: 35 - huE2-D16F
5′-CCTCTGACGTCCATCATCTC-3′
SEQ ID NO: 36 - feE2-D16R
5′-GGAGTGGGTGCAGTTGATGG-3′
SEQ ID NO: 37 - feE2-D16F
5′-CCTGTGACGTCCATCATTG-3′
SEQ ID NO: 38 - caE2-D16R
5′-GGAGTGGGTGCAGTTGATGG-3′
SEQ ID NO: 39 - caE2-D16F
5′-CCTGTGACATCCATCATTG-3′
HER2 Fragments
SEQ ID NO: 40 - p95 - LPESFDGDPASNTAP
SEQ ID NO: 41 - p93 - KIFGSLAPLPESFDG
SEQ ID NO: 42 - p94 - SLAFLPESFDGDPAS
SEQ ID NO: 43 - p96 - FDGDPASNTAPLQPE
SEQ ID NO: 44 - p97 - PASNTAPLQPEQLQV
SEQ ID NO: 45 - ESFDGDPASNT

PCR primers for construction of human ERBB2-delta16, feline ERBB2-delta16 and canine ERBB2-delta 16 and derivatives thereof.

| Sequences | |
|---|---|
| SEQ ID NO: 1 - Homo sapiens (Human; “E2ectm”) NP_004439.2 (1-687) | |
| 1 | melaalcrwg lllallppga astqvctgtd mklrlpaspe thldmlrhly qgcqvvqgnl |
| 61 | eltylptnas lsflqdiqev qgvvliahnq vrqvplqrlr ivrgtqlfed nvalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytm |
| SEQ ID NO: 2 - h(es)E2ectm (human) (1-687; with substitutions M198V, | |
| Q398R, F4251, H473R, A622T) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcvglgmehl revravtsan |
| 361 | igefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 3 - Pan paniscus (Bonobo) XP_0089590.53.1 (1-145, 170-711) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO:4 - Saimiri boliviensis boliviensis (Bolivian Squirrel Monkey) | |
| XP_010328997.1 (38-701) | |
| 38 | qvctgtdmkl rlpaspethl dmlrhlvqgc qvvqgnlelt ylptnaslsf lqdiqevqgy |
| 98 | vliahnqvrq vplqrlrivr gtqlfednya lavldngdpl dnttpvtgas pgglrelqlr |
| 158 | slteilkggv wiqrnpqlcy qdtilwkdif hknnqlaltl idtnrsrach pcspvckgsr |
| 218 | cwgessedcq sltrtvcagg carckgplpt dccheqcaag ctgpkhsdcl aclhfnhsgi |
| 278 | celhcpalvt yntdtfesmp npegrytfga scvtacpvny lstdvgsctl vcplhnqevt |
| 338 | aedgtqrcek cskpcarvcy glgmehlrev ravtsaniqe fvgckkifgs laflpesfdg |
| 398 | dpasntvplq peqlhvfetl eeitgylyis awpdslpdls vfqnlqvirg rilhngaysl |
| 458 | tlqglaiswl glrslrelgs glalihhnar lcfvhtvpwd nlfrnphqal lhtanrpehe |
| 518 | cvgkdlachp lcarghcwgp gptqcvncsq flrgqecvee crvlqglpre yvnarhclpc |
| 578 | hpecqpqngs vtcsgpeadq cvacahykds pfcvarcpsg vkpdisympi wkfpdeegtc |
| 638 | qpcpincths cvdlddkgcp aeqrasplts iisavvgill vmvlglllgi likrrqqkir |
| 698 | kytm |
| SEQ ID NO: 5 - Pan troglodytes (Chimp) XP_003315512.2 (1-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglamehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdek gtcqpcpinc thscvdlddk gcpaeqrasp ltsiisavvg |
| 661 | illvvvlgvv fgilikrrqq kirkytm |
| SEQ ID NO: 6 - Rhinopithecus roxellana (Gold Snooted No Monkey | |
| XP_010377602.1 (1-687) | |
| 1 | melaawcrwg lllallppga agtqvctgtd mklrlpaspe thldmlrhly qgcqvvqgnl |
| 61 | eltylptdas lsflqdiqev qgyvliahnq vrqvplqrlr ivrgtqlfed nyalavldng |
| 121 | dplnnitpvt gaspgglrel qlrslteilk ggvliqrnpq lcvqdtilwk difhknnqla |
| 181 | ftlidtnrsr achpcspvck gshcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskscar vcyglgmehl revravtsan |
| 361 | iqefagckki fgslaflpes fdgdlasnta plqpeqlrvf etleeitgyl yisawpdslp |
| 421 | dlsilqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 7 - Gorilla gorilla gorilla (Gorilla) XP_004041868.1 (1-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpe lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefaackki fgslaflpes fdgdpasnta plqpeqlqvf etleeitgyl yisawpdslp |
| 421 | dlsifqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytm |
| SEQ ID NO: 8 - Chlorocebus sabaeus (Green Monkey) XP_008011036.1 (1-687) | |
| 1 | melaawcswg lilallppga agtqvctgtd mklrlpaspe thldmlrhly qgcqvvqgnl |
| 61 | eltylptnas lsflqdiqev qgyvliahnq vrqvplqrlr ivrgtqlfed nvalavldng |
| 121 | dplnnttpvt gaspgglrel qlrslteilk ggvliqrnpq lcyqdtilwk difhknnqla |
| 181 | ltlidtnrsr achpcspack gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | vnylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgeala chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 9 - Papio anubis (Olive Baboon) XP_003912981.1 (1-687) | |
| 1 | melaawcrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynvlstdvgs ctlvcplhnq evtaedgtqr cekcskpcar vcyglgmehl revravtsan |
| 361 | iqefaackki fgslaflpes fdgdpasnta plqpeqlqvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 10 - Macaca mulatta (Rhesus) XP_001090430.1 (1-687) | |
| 1 | melaawyrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dllnnttpvt gaspgglrel qlrslteilk ggvliqrnpq lcyqdtilwk difhknnqla |
| 181 | ltlidtnrsr achpcspvck gsrcwgesse dcqsltrtvc aagcarckgp yptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqscpinc thscvdlddk gcpaeqrasp ltsiisavvg |
| 661 | illvvvlgvv fgilikrrqq kirkytm |
| SEQ ID NO: 11 - Macaca mulatta (Rhesus-HER2prot2) US7282365 B2 (#41) | |
| (1-687) | |
| 1 | melaawyrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcvqdtilwk difhknnqla |
| 181 | ltlidtnrsr achpcspvck gsrcwgesse dcqsitrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcaxghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqxasp ltsiisavvg |
| 661 | illvvvlgvv fgilikrrqq kirkytm |
| SEQ ID NO: 12 - Pongo abelii (Sumatran Orangutan) XP_009250137.1 | |
| (1-221, 229-666) | |
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliernpq lcyqdtilwk difhknnqla vtlidtnrls gxhpcffrcv raprcwgess |
| 181 | edcqsltrtv caggcarckg plptdccheq clpsxhgpqa psalpclhfn hsgicelhcp |
| 241 | alvtyntdtf esmpnpegry tfgascvtac pynylstdvg sctlvcplhn qevtaedgtq |
| 301 | rcekcskpca rvcyglgmeh lrevravtsa niqefagckk ifgslaflpe sfdgdpasnt |
| 361 | aplqpeqlrv fetleeitgy lyisawpdsl pdlsvfqnlq virgrilhng aysltlqglg |
| 421 | iswlglrslr elgsglalih hntrlcfvht vpwdqlfrnp hqallhtanr pedecvgegl |
| 481 | achqlcargh cwgpgptqcv ncsqflrgqe cveecrvlqg lpreyvnary clpchpecqp |
| 541 | qngsvtcfgp eadqcvacah vkdppfcvar cpsgvkpdls ympiwkfpde egtcqpcpin |
| 601 | cthscvdldd kgcpaeqras pltsiisavv gillvvvlgv vfgilikrrq qkirkytm |
| SEQ ID NO: 13 - Nomascus leucogenys (WhChGibbon) XP_003278275.1 (37-699) | |
| 37 | vctgtdmklr lpaspethld mlrhlyqgcq vvqgnlelty lptnaslsfl qdiqevqgyv |
| 97 | liahnqvrqv plqrlrivrg tqlfednyal avldngdpln nttlvtgasp gglrelqlrs |
| 157 | iteilkggvl iqrnpqlcyq dtilwkdifh knnqlaltli dtnrsracqp cspvckgsrc |
| 217 | wgessedcqs ltrtvcaggc arckgpiptd ccheqcaagc tapkhsdcla clhfnhsgic |
| 277 | elhcpalvty ntdtfesmpn pegrytfgas cvtacpynyl stdvgsctlv cplhnqevta |
| 337 | edgtqrcekc skpcarvcyg lgmehlrevr avtsaniqef agckkifgsl aflpesfdgd |
| 397 | pasntaplqp eqlqvfetle eitgylyisa wpdslsdlsv fqnlqvirgr ilhngayslt |
| 457 | iqglgiswlg lrslrelgsg lalihhnnrl cfvhtvpwdq ifrnphqall htanrpedec |
| 517 | vaeglachql carghcwgpg ptqcvncsqf lrgqecveec rvlqglprey vnarhclpch |
| 577 | pecqpqngsv tcfgpeadqc vscahykdpp fcvarcpsgv kpdlsvmpiw kfpdeegtcq |
| 637 | pcpincthsc vdlddkgcpa eqraspltsi isavvgillv vvlgavfgil ikrrqqkirk |
| 697 | ytm |
| SEQ ID NO: 14 - Callithrix jacchus (WhTufEarMarmoset) XP_002806904.1 | |
| (1-687) | |
| 1 | melaawcrwg llfallppga agtqvctatd mklrlpaspe thldmlrhly qgcqvvqgnl |
| 61 | eltylpanas lsflqdiqev qgyvliahnq vrqvplqrlr ivrgtqlfed nyalavldng |
| 121 | dpldnttpvt gaspgglrel qlrslteilk ggvwiqrnpq lcyqdmvlwk difhknnqla |
| 181 | ltlidtnrsr achpcspack gsrcwgesse dcqsltrtvc aggcarckgp lptdccheqc |
| 241 | aagctgpkhs dclacihfnh sgicelhcpa lvtyntdtfe smpnpegryt fgascvtacp |
| 301 | ynylstdvgs ctlvcplhnq evtaedgtqr cekcskpcar vcyglamehl revravtsan |
| 361 | iqefagckki fgslaflpes fdgdpasntv plqpeqlqvf etleeitgyl yisawpdslp |
| 421 | dlsvfqnlqv irgrilhnga ylltlqglgi swlglrslre lgsglalihh narlcfvhtv |
| 481 | pwdnlfrnph qallhtanrp ehecvgkdla chplcarghc wgpgptqcvn csqflrgqec |
| 541 | veecrvlqgl preyvnarhc lpchpecqpq ngsvtcsgpe adqcvacahy kdspfcvarc |
| 601 | psgvkpdlsv mpiwkfpdee gtcqpcpinc thscvdlddk gcpaeqrasp ltsiisavvg |
| 661 | illfmvlgll lgilmkrrqq kirkytm |
| SEQ ID NO: 15 - Macaca fascicularis (Macaca) XP_005584091.2 (98-784) | |
| 98 | melaawyrwg lllallppga tgtqvctgtd mklrlpaspe thldmlrhly qgcqvvqgnl |
| 158 | eltylptnas lsflqdiqev qgyvliahnq vrqvplqrlr ivrgtqlfed nyalavldng |
| 218 | dplnnttpvt gaspgglrel qlrslteilk ggvliqrnpq lcvqdtilwk difhknnqla |
| 278 | ltlidtnrsr achpcspvck gsrcwgesse dcqsltrtvc aggcarckgp lptdccheqc |
| 338 | aagctgpkhs dclaclhfnh sgicelhcpa lvtyntdtfe smpnpegryt fgascvtacp |
| 398 | vnylstdvgs ctlvcplhnq evtaedgtqr cekcskpcar vcyglgmehl revravtsan |
| 458 | igefagckki fgslaflpes fdgdpasnta plqpeqlrvf etleeitgyl yisawpdslp |
| 518 | dlsvlqnlqv irgrilhnga ysltlqglgi swlglrslre lgsglalihh ntrlcfvhtv |
| 578 | pwdqlfrnph qallhtanrp edecvgegla chqlcarghc wgpgptqcvn csqflrgqec |
| 638 | veecrvlqgl preyvnarhc lpchpecqpq ngsvtcfgpe adqcvacahy kdppfcvarc |
| 698 | psgvkpdlsv mpiwkfpdee gtcqscpinc thscvdlddk gcpaeqrasp ltsiisavvg |
| 758 | illvvvlgvv fgilikrrqq kirkytm |
| SEQ ID NO: 16 - Human ERBB2ectm-delta 16 NP_004439.2 (1-632, 649-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq icyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegrvt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thspltsiis avvgillvvv lgvvfgilik |
| 661 | rrqqkirkyt m |
| SEQ ID NO: 17 - Human (es)E2ectm-delta 16 (1-632, 649-687; with | |
| substitutions M198V, Q398R, F425L, H473R, A622T) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcvglamehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thspltsiis avvgillvvv lgvvfgilik |
| 661 | rrqqkirkyt m |
| SEQ ID NO: 18 - Feline E2ectm (wt) Translate of JN990983 (1-687) | |
| 1 | melaawcrwg lllallpsga tgtqvctgtd mklrlpaspe thldmlrhly qgcqvvqgnl |
| 61 | eltylhanas lsflqdiqev qgyvliahnq vkqvplqrlr ivrgtqlfed nyalavldng |
| 121 | dpldsgtpat gaalgglrel qlrslteilk ggvliqrnpq lchqdtilwk difhknnqla |
| 131 | lmlidtnrsr acqpcspack dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlrvf ealeeitgyl yisawpdslp |
| 421 | nlsvfqnlrv irgrvlhdga ysltlqglgi swlglrslre lgsglalihr nsrlcfvhtv |
| 481 | pwdqlfrnph qallhsanrp edecagegla cvplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gtcqpcpinc thscadldek gcpaeqrasp vtsiiaavvg |
| 661 | illvvvvglv lgilikrrrq kirkytm |
| SEQ ID NO: 19 - Feline (es)E2ectm (1-687; with substitutions A198V, | |
| R398Q, F425L, R473H, T622A) | |
| 1 | melaawcrwg lllallpsga tgtqvctgtd mklrlpaspe thldmlrhly qgcqvvqgnl |
| 61 | eltylhanas lsflqdiqev qgyvliahnq vkqvplqrlr ivrgtqlfed nyalavldng |
| 121 | dpldsgtpat gaalgglrel qlrslteilk ggvliqrnpq lchqdtilwk difhknnqla |
| 181 | lmlidtnrsr acqpcspvck dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlqvf ealeeitgyl yisawpdslp |
| 421 | nlsvlqnlrv irgrvlhdga ysltlqglgi swlglrslre lgsglalihr nshlcfvhtv |
| 481 | pwdqlfrnph qallhsanrp edecagegla cyplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gacqpcpinc thscadldek gcpaeqrasp vtsiiaavvg |
| 661 | illvvvvglv lgilikrrrq kirkytm |
| SEQ ID NO: 20 - Feline (es)E2ectm-delta16 Feline (es)E2ectm (1-632, | |
| 649-687) | |
| 1 | melaawcrwg lllallpsga tgtqvctgtd mklrlpaspe thldmlrhly qgcqvvqgnl |
| 61 | eltylhanas lsflqdiqev qgvvliahnq vkqvplqrlr ivrgtqlfed nvalavldng |
| 121 | dpldsgtpat gaalgglrel qlrslteilk ggvliqrnpq lchqdtilwk difhknnqla |
| 181 | lmlidtnrsr acqpcspvck dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlqvf ealeeitgyl yisawpdslp |
| 421 | nlsvlqnlrv irgrvlhdga ysltlqglgi swlglrslre lgsglalihr nshlcfvhtv |
| 431 | pwdqlfrnph qallhsanrp edecagegla cyplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gacqpcpinc thspvtsiia avvgillvvv vglvlgilik |
| 661 | rrrqkirkvt m |
| SEQ ID NO: 21 - Dog E2ectm(wt) NP_001003217 (1-687) | |
| 1 | melaawcrwg lllallpsga agtavctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dpleggipap gaapgglrel qlrslteilk ggvliqrspq lchqdtilwk dvfhknnqla |
| 131 | ltlidtnrsr acppcspack dahcwgassg dcqsltrtvc aggcarckgp qptdccheqc |
| 241 | aagctgpkhs dclaclhfnh sgicelhcpa lvtyntdtfe smpnpegryt fgascvtscp |
| 301 | ynylstdvgs ctlvcplnnq evtaedgtqr cekcskpcar vcvglgmehl revravtsan |
| 361 | igefagckki fgslaflpes fegdpasnta plqpeqlrvf ealeeitgyl yisawpdslp |
| 421 | nlsvfqnlrv irgrvlhdga ysltlqglgi swlglrslre lgsglalihr narlcfvhtv |
| 431 | pwdqlfrnph qallhsanrp eeecvgegla cyplcahghc wgpgptqcvn csqflrgqec |
| 541 | veecrvlqgl preyvkdryc lpchsecqpq nasvtcfgse adqcvacahy kdppfcvarc |
| 601 | psgvkpdlsf mpiwkfadee gtcqpcpinc thscadldek gcpaearasp vtsiiaavvg |
| 661 | illavvvglv lgilikrrrq kirkvtm |
| SEQ ID NO: 22 - Dog (es)E2ectm NP_001003217 (1-687; with substitutions | |
| A198V, R398Q, F425L, R473H, T622A) | |
| 1 | melaawcrwg lllallpsga agtqvctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dplegaipap gaapgglrel qlrslteilk ggvliqrspq lchqdtilwk dvfhknnqla |
| 181 | ltlidtnrsr acppcspvck dahcwgassg dcqsltrtvc aggcarckgp qptdccheqc |
| 241 | aagctgpkhs dclaclhfnh sgicelhcpa lvtvntdtfe smpnpegryt fgascvtscp |
| 301 | ynylstdvgs ctlvcplnnq evtaedgtqr cekcskpcar vcyglgmehl revravtsan |
| 361 | iqefagckki fgslaflpes fegdpasnta plqpeqlqvf ealeeitgyl yisawpdslp |
| 421 | nlsvlqnlrv irgrvlhdga ysltlqglgi swlglrslre lgsglalihr nahlcfvhtv |
| 481 | pwdqlfrnph qallhsanrp eeecvgegla cyplcahghc wgpgptqcvn csqflrgqec |
| 541 | veecrvlqgl preyvkdryc lpchsecqpq ngsvtcfgse adqcvacahy kdppfcvarc |
| 601 | psgvkpdlsf mpiwkfadee gacqpcpinc thscadldek gcpaeqrasp vtsiiaavvg |
| 661 | illavvvglv lgilikrrrq kirkvtm |
| SEQ ID NO: 23 - Dog (es)E2ectm-delta16 Dog (es)E2ectm (1-632, 649-687) | |
| 1 | melaawcrwg lllallpsga agtqvctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dpleggipap gaapgglrel qlrslteilk ggvliqrspq lchqdtilwk dvfhknnqla |
| 181 | ltlidtnrsr acppcspvck dahcwgassg dcqsltrtvc aggcarckgp qptdccheqc |
| 241 | aagctgpkhs dclaclhfnh sgicelhcpa lvtyntdtfe smpnpegryt fgascvtscp |
| 301 | ynylstdvgs ctlvcpinnq evtaedgtqr cekcskpcar vcyglgmehl revravtsan |
| 361 | iqefagckki fgslaflpes fegdpasnta plqpeqlqvf ealeeitgvl yisawpdslp |
| 421 | nlsvlqnlrv irgrvlhdga ysltlqglgi swlglrslre lgsglalihr nahlcfvhtv |
| 481 | pwdqlfrnph qallhsanrp eeecvgegla cyplcahghc wgpgptqcvn csqflrgqec |
| 541 | veecrvlqgl pryvvkdryc lpchsecqpq ngsvtcfgse adqcvacahy kdppfcvarc |
| 601 | psgvkpdlsf mpiwkfadee gacqpcpinc thspvtsiia avvgillavv vglvlgilik |
| 661 | rrrqkirkyt m |
| ERBB2 cDNAs for human, cat, dog; wt, (es), (es)d16 | |
| Technical note: The feline ERBB2 cDNAs, for example, are derived from | |
| accession #JN990983 (which inventors cloned). This has a 135 nt 5′ UTR, | |
| where the orf begins at nt 136. The numbering of the feline ERBB2 cDNA | |
| derivatives is based on this. In the case of (es) sequences, the sub- | |
| stitutions are indicated. Finally, since the ectm vaccines include only the | |
| first ~687 codons, a stop codon was added, as indicated. | |
| SEQ ID NO: 24 - Homo sapiens (Human; “E2ectm”) NM_004448 (1-2061, +TGA) | |
| 1 | atggagctgg cggccttgtg ccgctggggg ctcctcctcg ccctcttgcc ccccggagcc |
| 61 | gcgaacaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | aacccgctaa acaataccac ccctgtcaca ggggcctccc caggaggcct gcgggagcta |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc gatgtgtaag |
| 601 | ggctcccgct gctggggaga gagttctgag gattgtcaga gcctgacgcg cactgtctgt |
| 661 | gccgatggct gtgcccgcta caagggacca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccatgccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | aaggtgacag cagagaatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatgggg acccagcctc caacactgcc ccgctccagc cagagcagct ccaagtgttt |
| 1201 | gagactctgg aagagatcac aggttaccta tacatctcag catggccgga cagcctgcct |
| 1261 | gacctcagcg tcttccagaa cctgcaagta atccggggac aaattctgca caatgacgcc |
| 1321 | tactcgctga ccctgcaagg gctgggcatc agctggctgg ggctgcgctc actgagggaa |
| 1381 | ctgggcagtg gactggccct catccaccat aacacccacc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcgcatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctgg tcgtggtctt gggggtagtc tttaggatcc tcatcaagcg acggcagcag |
| 2041 | aagatccgga agtacacgat gtga |
| SEQ ID NO: 25 -h(es)E2tm (human) | |
| 1 | atggagctgg cggccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | aaactcacct acctgcccac caatgccagc ctgtccttcc tgcaagatat ccaggaggta |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctactacc aagacacgat tttgtgaaag gacatcttcc acaagaacaa ccagctggct |
| 541 | ctcacactga tagacaccaa ccgctctcgg gcctgccacc cctgttctcc ggtgtgtaag |
| 601 | ggctcccgct gctggggaga gagttctgag gattgtcaga gcctgacgcg cactgtctgt |
| 661 | gccggtggct gtgcccgctg caaggggcca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgaa |
| 841 | tccatgccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | gaggtgacag cagaggatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggcta caagaaaatc tttgggagcc tggcatttct gccggagagc |
| 1141 | tttgatgggg acccagcctc caacactgcc ccgctccagc cagagcagct cagagtgttt |
| 1201 | gagactctgg aagagatcac aggttaccta tacatctcag catggccgga cagcctgcct |
| 1261 | gacctcagcg tcctccagaa cctgcaagta atccggggac gaattctgca caatggcgcc |
| 1321 | tactcgctga ccctgcaagg gctgggcatc agctggctgg ggctgcgctc actgagggaa |
| 1381 | ctgggcagtg gactggccct catccaccat aacacccgcc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagta tcagccccag aatagctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcacatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctag tcgtgatctt ggaggtggtc tttgggatcc tcatcaagcg acggcagcaa |
| 2041 | aagatccgga agtacacgat gtaa |
| SEQ ID NO:26 - Human E2ectm-delta 16 NM_004448 (1-1898, 1947-2061, +TAA) | |
| 1 | atggagctgg cggccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctcca ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc gatgtgtaag |
| 601 | ggctcccgct gctgggaaga gagttctgag gattgtcaga gcctgacgcg cactgtctgt |
| 661 | gccgatggct gtgcccgcta caaggggcca ctgcccacta actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccataccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | aaggtgacag cagagaatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatggga acccagcctc caacactgcc ccgctccagc cagagcagct ccaagtgttt |
| 1201 | gagactctgg aagagatcac aggttaccta tacatctcag catggccgga cagcctgcct |
| 1261 | gacctcagcg tcttccagaa cctgcaagta atccggggac gaattctgca caatggcgcc |
| 1321 | tactcgctga ccctgcaagg gctgggcatc agctggctgg ggctgcgctc actgagggaa |
| 1381 | ctgggcagtg gactggccct catccaccat aacacccacc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcgcatgcc aaccttgccc catcaactgc acccactccc ctctgacgtc catcatctct |
| 1921 | gcggtggttg gcattctgct ggtcgtggtc ttgggggtgg tctttgggat cctcatcaag |
| 1981 | cgacggcagc agaagatccg gaagtacacg atgtaa |
| SEQ ID NO: 27 - Human (es)E2ectm-delta 16 | |
| 1 | atggaactgg cgaccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc ggtgtgtaag |
| 601 | ggctcccgct gctggggaga gagttctgag gattgtcaga gcctgacgcg cactgtctgt |
| 661 | gccggtggct gtgcccgctg caaggggcca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccatgccca atcccgaggg ccggtataca ttcggcgcca gctgtgtgac tgcctgtccc |
| 901 | tacaactacc tttctacgga cgtaggatcc tgcaccctcg tctgccccct gcacaaccaa |
| 961 | gaggtgacag cagaggatgg aacacagcgg tgtgagaagt gcagcaagcc ctatgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatgggg acccagcctc caacactgcc ccgctccagc cagagcagct cagagtgttt |
| 1201 | gagactctgg aaaagatcac aggttaccta tacatctcag catggccgga cagcctgcct |
| 1261 | gacctcagcg tcctccagaa cctgcaaata atccggggac gaattctgca caatggcgcc |
| 1321 | tactcgctga ccctgcaagg gctgggcatc agctggctgg ggctgcgctc actgaggaaa |
| 1381 | ctgggcagtg gactggccct catccaccat aacacccgcc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaagggctc cccagggagt atgtgaatgc caagcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcacatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctgg tcgtggtctt gggggtggtc tttgggatcc tcatcaagcg acggcagcag |
| 2041 | aagatccgga agtacacgat gtaa |
| SEQ ID NO: 28 - Feline E2ectm (wt) JN990983 (136-2196, +TGA) | |
| 1 | atggagctgg cggcctggtg ccgctggggg ctcctcctcg ccctcctgcc ctccggagcc |
| 61 | acgggcaccc aagtgtacac cggcacagac atgaagctgc ggctcccagc cagtcccgag |
| 121 | acccacctgg acatgctccg ccacctctac cagggctgtc aagtgataca gggcaacctg |
| 181 | gagctcacct acctgcatgc caatgccagc ctctccttcc tgcaggatat ccaggaggtg |
| 241 | caaggctatg tgctcattgc ccacaaccaa gtgaaacagg tcccactgca gaggctacga |
| 301 | atcgtgcgag gcacccagct ctttgaggac aactacgccc tggccgtgct ggacaacgga |
| 361 | gacccactgg acagtggcac ccctgctaca ggggctgccc taggagggct gcgggagctg |
| 421 | cagctccgaa gcctcacaga gatcctgaag ggaggggtcc tcattcagcg gaacccgcag |
| 481 | ctctgccacc aggacacgat tctgtggaag gacatcttcc acaagaacaa ccagctggcc |
| 541 | ctcatgctga tagacaccaa ccgctctcgg gcctgccaac cctgttctcc agcttgtaaa |
| 601 | gactcccact gctggggagc aagttccggg gactgtcaga gcttgactcg aactgtctgt |
| 661 | gctggcggct gtgcccgctg caagggcccg cagcccaccg actgctgcca cgagcaatgt |
| 721 | gctgctggct gcacgggccc caagcattct gactgcctgg cctgcctcca cttcaaccac |
| 781 | agtggcatct gtgagctgca ctgcccagcc ctggtcacct acaacacgga caccttcgaa |
| 841 | tccatgccca accctgaggg ccgttatacc ttcggtgcca gctgtgtgac tgcctgtccc |
| 901 | tacaactacc tgtctacgga cgtgggatcc tgcaccctgg tctgtcccct gaacaaccaa |
| 961 | gaggtaacag ctaaggatgg aacacagcgg tgtgagaaat gcagcaagcc ctgtgcccga |
| 1021 | gtgtgctacg gcctaggcat ggagcacctg cgggaggcga gggcagtcac cagtgccaac |
| 1081 | atccaagaat ttgtcggctg caagaagatc tttgggagcc tggcgtttct gccagagagc |
| 1141 | tttgaggggg acccagcctc caacactgcc cccctgcagc ctgagcagct cagagtgttt |
| 1201 | gaggctctgg aggagattac aggttacctg tacatctcag cgtggccaga cagcttgcct |
| 1261 | aacctcagtg tcttccagaa cctcagagtg atccggggcc gagttctgca tgacggtgct |
| 1321 | tactcgctga cccttcaagg gctgggcatc agctggctgg ggctgcgctc gctgggggag |
| 1381 | ctggacagtg gactggccct catccaccgc aactcccgcc tctgcttcgt acacacggtg |
| 1441 | ccctgggacc agctcttccg gaacccccac caggccctgc tccacagcgc caaccggcca |
| 1501 | gaggacgagt gcgcgggtga gggcctggcc tgctatccgc tgtgtgccca cgggcactgc |
| 1561 | tggggtccgg gacccaccca gtgtgtcaac tgcagccagt tccttcgggg ccaggagtgc |
| 1621 | gtggaggaat gccgagtatt gcaggggctt ccccgggagt atgtgaagga taggttctgt |
| 1681 | ctgccatgcc acccgaagtg tcagccccag aatggctcag tgacctgctt gggctcggaa |
| 1741 | gctgaccagt gtgtggcctg tgcccactac aaggaccctc ctttctgtgt ggctcgctgc |
| 1801 | cccagtgggg tgaaacctga cctctccttc atgcccatct ggaagttcgc agatgaggag |
| 1861 | ggcacgtgcc agccatgccc catcaactgc acccactcct gtgcgaacct ggacgagaag |
| 1921 | ggctgccccg ccgagcagag agccagccct atgacgtcca tcattgctgc tgtggtgggc |
| 1981 | attctgctgg tcgtggttgt ggggctggtc cttggcatcc taatcaagcg aaggcggcag |
| 2041 | aagatccgga agtacacgat gtga |
| SEQ ID NO: 29 - Feline (es)ERBB2ectm JN990983 (136-2196; with substitutions | |
| c728t, t729g, a1327c, g1328a, a1329g, t1408c, g1553a, a1999g, g2001c; taa) | |
| 1 | gtacaagaat gaagttgtgg agctgagagt cccctgcgtc gtgccccgag agccgaacag |
| 61 | agctcccagg cagccgcccg gcccttcgca gcccggtcca gcccgagcca tggggccgga |
| 121 | gccgcagtga gcaccatgga gctggcggcc tggtgccgct gggggctcct cctcgccctc |
| 181 | ctgccctccg gagccacggg cacccaagtg tgcaccggca cagacatgaa gctgcggctc |
| 241 | ccagccagtc ccgagaccca cctggacatg ctccgccacc tctaccaggg ctgtcaagtg |
| 301 | gtacagggca acctggagct cacctacctg catgccaatg ccagcctctc cttcctgcag |
| 361 | gatatccagg aggtgcaagg ctatgtgctc attgcccaca accaagtgaa acaggtccca |
| 421 | ctgcagaggc tacgaatcgt gcgaggcacc cagctctttg aggacaacta cgccctggcc |
| 481 | gtgctggaca acggagaccc actggacagt ggcacccctg ctacaggggc tgccctagga |
| 541 | gggctgcggg agctgcagct ccgaagcctc acagagatcc tgaagggagg ggtcctcatt |
| 601 | cagcggaacc cgcagctctg ccaccaggac acgattctgt ggaaggacat cttccacaag |
| 661 | aacaaccagc tggccctcat gctgatagac accaaccgct ctcgggcctg ccaaccctgt |
| 721 | tctccagtgt gtaaagactc ccactgctgg ggagcaagtt ccggggactg tcagagcttg |
| 781 | actcgaactg tctgtgctgg cggctgtgcc cgctgcaagg gcccgcagcc caccgactgc |
| 841 | tgccacgagc aatgtgctgc tggctgcacg ggccccaagc attctgactg cctggcctgc |
| 901 | ctccacttca accacagtgg catctgtgag ctgcactgcc cagccctggt cacctacaac |
| 961 | acggacacct tcgaatccat gcccaaccct gagggccgtt ataccttcgg tgccagctgt |
| 1021 | gtgactgcct gtccctacaa ctacctgtct acggacgtgg gatcctgcac cctggtctgt |
| 1081 | cccctgaaca accaagaggt gacagctgag gatggaacac agcggtgtga gaaatgcagc |
| 1141 | aagccctgtg cccgagtgtg ctacggccta ggcatggagc acctgcggga ggcgagagca |
| 1201 | gtcaccagtg ccaacatcca agaatttgtc ggctgcaaga agatctttgg gagcctggcg |
| 1261 | tttctgccag agagctttga gggggaccca gcctccaaca ctgcccccct gcagcctgag |
| 1321 | cagctccagg tgtttgaggc tctggaggag attacaggtt acctgtacat ctcagcgtgg |
| 1381 | ccagacagct tgcctaacct cagtgtcctc cagaacctca gagtgatccg ggaccgagtt |
| 1441 | ctgcatgacg gtgcttactc gctaaccctt caagggctgg gcatcagctg gctggggctg |
| 1501 | cgctcgctgc gagagctggg cagtgggctg gccctcatcc accgcaactc ccacctctgc |
| 1561 | ttcgtacaca cggtgccctg ggaccagctc ttccggaacc cccaccaggc cctgctccac |
| 1621 | agcgccaacc ggccagagga cgagtgcgcg ggtgagggcc tggcctgcta tccgctgtgt |
| 1681 | gcccacgggc actgctgggg tccgggaccc acccagtgtg tcaactgcag ccagttcctt |
| 1741 | cggggccagg agtgcgtgga ggaatgccga gtattgcagg gacttccccg ggagtatgtg |
| 1801 | aaggataggt tctgtctgcc atgccacccg gagtgtcagc cccaaaatgg ctcagtgacc |
| 1861 | tgcttgggct cggaagctga ccagtgtgtg gcctgtgccc actacaagga ccctcctttc |
| 1921 | tgtgtggctc gctgccccag tggggtgaaa cctgacctct ccttcatgcc catctggaag |
| 1981 | ttcgcagatg aggaggacgc ctgccagcca tgccccatca actgcaccca ctcctgtgcg |
| 2041 | gacctggacg agaagggctg ccccgccgag cagagagcca gccctgtgac gtccatcatt |
| 2101 | gctgctgtgg tgggcattct gctggtcgtg gttgtggggc tggtccttgg catcctaatc |
| 2161 | aagcgaaggc ggcagaagat ccggaagtac acgatgtaa |
| SEQ ID NO: 30 - Feline (es)ERBB2ectm-delta16 Feline (es)ERBB2ectm | |
| (136-2033, 2082-2196; taa) | |
| 1 | gtacaagaat gaagttgtgg agctgagagt cccctgcgtc gtgccccgag agccgaacag |
| 61 | agctcccagg cagccgcccg gcccttcgca gcccggtcca gcccaagcca tggggccgga |
| 121 | gccgcagtaa gcaccatgga gctggcggcc tggtgccgct ggggactcct cctcgccctc |
| 181 | ctgccctccg gagccacggg cacccaagtg tgcaccggca cagacatgaa gctgcggctc |
| 241 | ccagccagtc ccgagaccca cctggacatg ctccgccacc tctaccaggg ctgtcaagtg |
| 301 | gtacagggca acctggagct cacctacctg catgccaatg ccagcctctc cttcctgcag |
| 361 | gatatccagg aagtgcaaga ctatgtgctc attgcccaca accaagtgaa acaggtccca |
| 421 | ctgcagaggc tacgaatcgt gcgaggcacc cagctctttg aggacaacta cgccctggcc |
| 481 | gtgctggaca acggagaccc actggacagt ggcacccctg ctacaggggc tgccctagga |
| 541 | gggctgcggg agctgcagct ccgaagcctc acagagatcc tgaagggagg ggtcctcatt |
| 601 | cagcggaacc cgcagctctg ccaccagaac acgattctgt gaaaggacat cttccacaag |
| 661 | aacaaccagc tggccctcat gctgatagac accaaccgct ctcgggcctg ccaaccctgt |
| 721 | tctccagtgt gtaaagactc ccactgctgg ggagcaagtt ccggggactg tcagagcttg |
| 781 | actcgaacta tctgtgctgg cggctgtgcc cgctgcaagg gcccgcagcc caccgactgc |
| 841 | taccacgagc aatgtgctgc tggctgcacg ggccccaagc attctagctg cctggcctgc |
| 901 | ctccacttca accacagtga catctgtgag ctgcactgcc cagccctggt cacctacaac |
| 961 | acggacacct tcgaatccat gcccaaccct gagggccgtt ataccttcgg tgccagctgt |
| 1021 | gtgactgcct gtccctacaa ctacctgtct acggacgtgg gatcctgcac cctggtctgt |
| 1081 | cccctaaaca accaagaggt gacagctgag gatggaacac agcggtgtga gaaatgcagc |
| 1141 | aagccctgtg cccgagtgtg ctacggccta ggcatggagc acctgcggga ggcgagggca |
| 1201 | gtcaccagtg ccaacatcca agaatttgtc ggctgcaaga agatctttgg gagcctggcg |
| 1261 | tttctgccag agagctttga gggggaccca gcctccaaca ctgcccccct gcagcctgag |
| 1321 | cagctccagg tgtttgaggc tctggaggag attacaggtt acctgtacat ctcagcgtgg |
| 1381 | ccagacagct tgcctaacct cagtgtcctc cagaacctca gagtgatccg gggccgagtt |
| 1441 | ctgcatgaca gtgcttactc gctgaccctt caagggctgg gcatcagctg gctggggctg |
| 1501 | cgctcgctgc gggagctggg cagtgggctg gccctcatcc accgcaactc ccacctctgc |
| 1561 | ttcgtacaca cggtgccctg ggaccagctc ttccggaacc cccaccaggc cctgctccac |
| 1621 | agcgccaacc ggccagagga cgagtgcgcg ggtgagggcc tggcctgcta tccgctgtgt |
| 1681 | gcccacgggc actgctgggg tccgggaccc acccagtgtg tcaactgcag ccagttcctt |
| 1741 | cggggccagg agtgcgtgga ggaatgccga gtattgcagg gacttccccg ggagtatgtg |
| 1801 | aaggataggt tctgtctgcc atgccacccg gagtgtcagc cccagaatgg ctcagtgacc |
| 1861 | tgcttgggct cggaagctga ccagtgtgtg gcctgtgccc actacaagga ccctcctttc |
| 1921 | tgtgtggctc gctgccccag tggggtgaaa cctgacctct ccttcatgcc catctggaag |
| 1981 | ttcgcagatg aggaggacgc ctgccagcca tgccccatca actgcaccca ctcccctgtg |
| 2041 | acgtccatca ttgctgctgt ggtgggcatt ctgctggtcg tggttgtggg gctggtcctt |
| 2101 | ggcatcctaa tcaagcgaag gcggcagaag atccggaagt acacgatgta a |
| SEQ ID NO: 31 - Canine E2ectm (wt) NM_001003217 (1-2058, +TGA) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aaatggtaca ggggaacctg |
| 181 | gagctcactt acctgcctgc caatgccagc ctgtccttcc tgcaggatat ccaggaggtg |
| 241 | cagggctatg tgctcattgc tcacagccaa atgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | caacttcgaa gcctcacaga gatcctgaag gaaggggtct tgattcagcg gagcccgcag |
| 481 | ctctgccacc aggacacgat tttatggaag gacgtcttcc ataagaacaa ccaactggcc |
| 541 | ctcacgctga tagacaccaa ccgcttttcg gcctacccgc cctgttctcc agcttgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgagaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttaatgggg acccagcctc caacactgcc cccctacagc ctgagcagct cagagtgttt |
| 1201 | gaggctctgg aggagatcac aggttacctg tacatctcag cgtggccaga cagcctgcct |
| 1261 | aacctcagtg tcttccagaa cctgcgagta atccggggac gagttctgca tgatggtgcc |
| 1321 | tactcgctga ccctgcaagg gctgggcatc agctgactgg ggctgcgctc gctgcggaaa |
| 1381 | ctgggcagtg ggctggccct catccaccgc aacgcccgcc tttgcttcgt gcacacggtg |
| 1441 | ccctgggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggaatgcgtg |
| 1621 | gaagaatgcc gagtactgca ggggctgccc cgagagtatg tgaaggacag gtactgtcta |
| 1681 | ccgtgccact cagagtgtca gccccagaat ggctcagtga cctgtttcgg atcggaagct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | acttgccagc cgtaccccat caactgcacc cactcctgtg cgaacctgga cgagaagggc |
| 1921 | tgtcccgccg agcagagagc cagccctgtg acatccatca ttaccgctgt ggtgggcatt |
| 1981 | ctgctggctg tggtcgtggg gctggtcctc ggcatcctga tcaagcgaag gcggcagaag |
| 2041 | atccggaagt acactatgtg a |
| SEQ ID NO: 32 - Canine (es)E2ectm NM _001003217 (1-2058, +TGA; with | |
| substitutions c593t, t594g, a1192c, g1193a, a1194g, t1273c, g1418a, | |
| a1861g, t1863c)) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aagtggtaca gggaaacctg |
| 181 | gagctcactt acctgcctgc caatgccagc ctgtccttcc tacaggatat ccaggaagtg |
| 241 | cagggctatg tgctcattgc tcacagccaa gtgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | cagcttcgaa gcctcacaga gatcctgaag ggaggggtct tgattcagcg gagcccgcag |
| 481 | ctctgccacc aggacacgat tttatggaag gacgtcttcc ataagaacaa ccagctggcc |
| 541 | ctcacgctga tagacaccaa ccgcttttcg gcctgcccgc cctgttctcc agtgtgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgaaaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttgatgggg acccagcctc caacactgcc cccctacagc ctgagcagct ccaggtgttt |
| 1201 | gaggctctgg aggagatcac aggttacctg tacatctcag cgtggccaga cagcctgcct |
| 1261 | aacctcagtg tcctccagaa cctgcgagta atccggggac gagttctgca tgatggtgcc |
| 1321 | tactcgctga ccctgcaagg gctgggcatc agctggctgg ggctgcgctc gctgcgggaa |
| 1381 | ctgggcagtg ggctggccct catccaccgc aacgcccacc tttgcttcgt gcacacggtg |
| 1441 | ccctgggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggagtgcgtg |
| 1621 | gaggaatgcc gagtactgca ggggctgccc cgagaatatg tgaaggacag gtactgtcta |
| 1681 | ccgtgccact cagagtatca gccccagaat ggctcagtga cctgtttcgg atcggaggct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | gcctgccagc cgtgccccat caactgcacc cactcctgtg cggacctgga cgaaaagggc |
| 1921 | tgtcccgccg agcagagagc cagccctgtg acatccatca ttgccgctgt ggtaggcatt |
| 1981 | ctgctggctg tggtcgtggg gctggtcctc ggcatcctga tcaagcgaag gcggcagaag |
| 2041 | atccggaagt acactatgtg a |
| SEQ ID NO: 33 - Dog (es)E2ectm-deltal6 Dog (es)E2ectm (with 1896-1943 | |
| deleted) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aagtggtaca ggggaacctg |
| 181 | gagctcactt acctgcctgc caatgccagc ctgtccttcc tgcaggatat ccaggaggtg |
| 241 | cagggctatg tgctcattgc tcacagccaa gtgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | cagcttcgaa gcctcacaga gatcctgaag ggaggggtct tgattcagcg gagcccgcag |
| 481 | ctctgccacc aggacacgat tttatggaag gacgtcttcc ataagaacaa ccagctggcc |
| 541 | ctcacgctga tagacaccaa ccgcttttcg gcctgcccgc cctgttctcc agtgtgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgagaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttgatgggg acccagcctc caacactgcc cccctacagc ctgagcagct ccaggtgttt |
| 1201 | gaggctctgg aggagatcac aggttacctg tacatctcag cgtggccaga cagcctgcct |
| 1261 | aacctcagtg tcctccagaa cctgcgagta atccggggac gagttctgca tgatggtgcc |
| 1321 | tactcgctga ccctgcaagg gctgggcatc agctggctgg ggctgcgctc gctgcgggaa |
| 1381 | ctgggcagtg ggctggccct catccaccgc aacgcccacc tttgcttcgt gcacacggtg |
| 1441 | ccctaggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggagtgcgtg |
| 1621 | gaggaatgcc gagtactgca ggggctgccc cgagagtatg tgaaggacag gtactgtcta |
| 1681 | ccgtgccact cagagtgtca gccccagaat ggctcagtga cctgtttcgg atcggaggct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | gcctgccagc cgtgccccat caactgcacc cactcccctg tgacatccat cattgccgct |
| 1921 | gtggtgggca ttctgctggc tgtggtcgtg gggctggtcc tcggcatcct gatcaagcga |
| 1981 | aggcggcaga agatccggaa gtacactatg tga |

PCR primers for construction of human ERBB2-delta16, feline ERBB2-delta16
and canine ERBB2-delta16 and derivatives thereof.
SEQ ID NO: 34 - huE2-D16R
5′-GGAGTGGGTGCAGTTGATGG-3′
SEQ ID NO: 35 - huE2-D16F
5′-CCTCTGACGTCCATCATCTC-3′
SEQ ID NO: 36 - feE2-D16R
5′-GGAGTGGGTGCAGTTGATGG-3′
SEQ ID NO: 37 - feE2-D16F
5′-CCTGTGACGTCCATCATTG-3′
SEQ ID NO: 38 - caE2-D16R
5′-GGAGTGGGTGCAGTTGATGG-3′
SEQ ID NO: 39 - caE2-D16F
5′-CCTGTGACATCCATCATTG-3′
HER2 Fragments
SEQ ID NO: 40 - p95 - LPESFDGDPASNTAP
SEQ ID NO: 41 - p93 - KIFGSLAPLPESFDG
SEQ ID NO: 42 - p94 - SLAFLPESFDGDPAS
SEQ ID NO: 43 - p96 - FDGDPASNTAPLQPE
SEQ ID NO: 44 - p97 - PASNTAPLQPEQLQV
SEQ ID NO: 45 - ESFDGDPASNT

### HER2 Fragments

| Sequences | |
|---|---|
| SEQ ID NO: 1 - Homo sapiens (Human; “E2ectm”) NP_004439.2 (1-687) | |
| 1 | melaalcrwg lllallppga astqvctgtd mklrlpaspe thldmlrhly qgcqvvqgnl |
| 61 | eltylptnas lsflqdiqev qgvvliahnq vrqvplqrlr ivrgtqlfed nvalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytm |
| SEQ ID NO: 2 - h(es)E2ectm (human) (1-687; with substitutions M198V, | |
| Q398R, F4251, H473R, A622T) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcvglgmehl revravtsan |
| 361 | igefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 3 - Pan paniscus (Bonobo) XP_0089590.53.1 (1-145, 170-711) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO:4 - Saimiri boliviensis boliviensis (Bolivian Squirrel Monkey) | |
| XP_010328997.1 (38-701) | |
| 38 | qvctgtdmkl rlpaspethl dmlrhlvqgc qvvqgnlelt ylptnaslsf lqdiqevqgy |
| 98 | vliahnqvrq vplqrlrivr gtqlfednya lavldngdpl dnttpvtgas pgglrelqlr |
| 158 | slteilkggv wiqrnpqlcy qdtilwkdif hknnqlaltl idtnrsrach pcspvckgsr |
| 218 | cwgessedcq sltrtvcagg carckgplpt dccheqcaag ctgpkhsdcl aclhfnhsgi |
| 278 | celhcpalvt yntdtfesmp npegrytfga scvtacpvny lstdvgsctl vcplhnqevt |
| 338 | aedgtqrcek cskpcarvcy glgmehlrev ravtsaniqe fvgckkifgs laflpesfdg |
| 398 | dpasntvplq peqlhvfetl eeitgylyis awpdslpdls vfqnlqvirg rilhngaysl |
| 458 | tlqglaiswl glrslrelgs glalihhnar lcfvhtvpwd nlfrnphqal lhtanrpehe |
| 518 | cvgkdlachp lcarghcwgp gptqcvncsq flrgqecvee crvlqglpre yvnarhclpc |
| 578 | hpecqpqngs vtcsgpeadq cvacahykds pfcvarcpsg vkpdisympi wkfpdeegtc |
| 638 | qpcpincths cvdlddkgcp aeqrasplts iisavvgill vmvlglllgi likrrqqkir |
| 698 | kytm |
| SEQ ID NO: 5 - Pan troglodytes (Chimp) XP_003315512.2 (1-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglamehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdek gtcqpcpinc thscvdlddk gcpaeqrasp ltsiisavvg |
| 661 | illvvvlgvv fgilikrrqq kirkytm |
| SEQ ID NO: 6 - Rhinopithecus roxellana (Gold Snooted No Monkey | |
| XP_010377602.1 (1-687) | |
| 1 | melaawcrwg lllallppga agtqvctgtd mklrlpaspe thldmlrhly qgcqvvqgnl |
| 61 | eltylptdas lsflqdiqev qgyvliahnq vrqvplqrlr ivrgtqlfed nyalavldng |
| 121 | dplnnitpvt gaspgglrel qlrslteilk ggvliqrnpq lcvqdtilwk difhknnqla |
| 181 | ftlidtnrsr achpcspvck gshcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskscar vcyglgmehl revravtsan |
| 361 | iqefagckki fgslaflpes fdgdlasnta plqpeqlrvf etleeitgyl yisawpdslp |
| 421 | dlsilqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 7 - Gorilla gorilla gorilla (Gorilla) XP_004041868.1 (1-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpe lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefaackki fgslaflpes fdgdpasnta plqpeqlqvf etleeitgyl yisawpdslp |
| 421 | dlsifqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytm |
| SEQ ID NO: 8 - Chlorocebus sabaeus (Green Monkey) XP_008011036.1 (1-687) | |
| 1 | melaawcswg lilallppga agtqvctgtd mklrlpaspe thldmlrhly qgcqvvqgnl |
| 61 | eltylptnas lsflqdiqev qgyvliahnq vrqvplqrlr ivrgtqlfed nvalavldng |
| 121 | dplnnttpvt gaspgglrel qlrslteilk ggvliqrnpq lcyqdtilwk difhknnqla |
| 181 | ltlidtnrsr achpcspack gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | vnylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgeala chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 9 - Papio anubis (Olive Baboon) XP_003912981.1 (1-687) | |
| 1 | melaawcrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynvlstdvgs ctlvcplhnq evtaedgtqr cekcskpcar vcyglgmehl revravtsan |
| 361 | iqefaackki fgslaflpes fdgdpasnta plqpeqlqvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqrasp ltsiisavvg |
| 661 | illvvvlgvv fgilikrrqq kirkytm |
| SEQ ID NO: 10 - Macaca mulatta (Rhesus) XP_001090430.1 (1-687) | |
| 1 | melaawyrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dllnnttpvt gaspgglrel qlrslteilk ggvliqrnpq lcyqdtilwk difhknnqla |
| 181 | ltlidtnrsr achpcspvck gsrcwgesse dcqsltrtvc aagcarckgp yptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqscpinc thscvdlddk gcpaeqrasp ltsiisavvg |
| 661 | illvvvlgvv fgilikrrqq kirkytm |
| SEQ ID NO: 11 - Macaca mulatta (Rhesus-HER2prot2) US7282365 B2 (#41) | |
| (1-687) | |
| 1 | melaawyrwg lllallppga agtqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcvqdtilwk difhknnqla |
| 181 | ltlidtnrsr achpcspvck gsrcwgesse dcqsitrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcaxghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thscvdlddk gcpaeqxasp ltsiisavvg |
| 661 | illvvvlgvv fgilikrrqq kirkytm |
| SEQ ID NO: 12 - Pongo abelii (Sumatran Orangutan) XP_009250137.1 | |
| (1-221, 229-666) | |
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliernpq lcyqdtilwk difhknnqla vtlidtnrls gxhpcffrcv raprcwgess |
| 181 | edcqsltrtv caggcarckg plptdccheq clpsxhgpqa psalpclhfn hsgicelhcp |
| 241 | alvtyntdtf esmpnpegry tfgascvtac pynylstdvg sctlvcplhn qevtaedgtq |
| 301 | rcekcskpca rvcyglgmeh lrevravtsa niqefagckk ifgslaflpe sfdgdpasnt |
| 361 | aplqpeqlrv fetleeitgy lyisawpdsl pdlsvfqnlq virgrilhng aysltlqglg |
| 421 | iswlglrslr elgsglalih hntrlcfvht vpwdqlfrnp hqallhtanr pedecvgegl |
| 481 | achqlcargh cwgpgptqcv ncsqflrgqe cveecrvlqg lpreyvnary clpchpecqp |
| 541 | qngsvtcfgp eadqcvacah vkdppfcvar cpsgvkpdls ympiwkfpde egtcqpcpin |
| 601 | cthscvdldd kgcpaeqras pltsiisavv gillvvvlgv vfgilikrrq qkirkytm |
| SEQ ID NO: 13 - Nomascus leucogenys (WhChGibbon) XP_003278275.1 (37-699) | |
| 37 | vctgtdmklr lpaspethld mlrhlyqgcq vvqgnlelty lptnaslsfl qdiqevqgyv |
| 97 | liahnqvrqv plqrlrivrg tqlfednyal avldngdpln nttlvtgasp gglrelqlrs |
| 157 | iteilkggvl iqrnpqlcyq dtilwkdifh knnqlaltli dtnrsracqp cspvckgsrc |
| 217 | wgessedcqs ltrtvcaggc arckgpiptd ccheqcaagc tapkhsdcla clhfnhsgic |
| 277 | elhcpalvty ntdtfesmpn pegrytfgas cvtacpynyl stdvgsctlv cplhnqevta |
| 337 | edgtqrcekc skpcarvcyg lgmehlrevr avtsaniqef agckkifgsl aflpesfdgd |
| 397 | pasntaplqp eqlqvfetle eitgylyisa wpdslsdlsv fqnlqvirgr ilhngayslt |
| 457 | iqglgiswlg lrslrelgsg lalihhnnrl cfvhtvpwdq ifrnphqall htanrpedec |
| 517 | vaeglachql carghcwgpg ptqcvncsqf lrgqecveec rvlqglprey vnarhclpch |
| 577 | pecqpqngsv tcfgpeadqc vscahykdpp fcvarcpsgv kpdlsvmpiw kfpdeegtcq |
| 637 | pcpincthsc vdlddkgcpa eqraspltsi isavvgillv vvlgavfgil ikrrqqkirk |
| 697 | ytm |
| SEQ ID NO: 14 - Callithrix jacchus (WhTufEarMarmoset) XP_002806904.1 | |
| (1-687) | |
| 1 | melaawcrwg llfallppga agtqvctatd mklrlpaspe thldmlrhly qgcqvvqgnl |
| 61 | eltylpanas lsflqdiqev qgyvliahnq vrqvplqrlr ivrgtqlfed nyalavldng |
| 121 | dpldnttpvt gaspgglrel qlrslteilk ggvwiqrnpq lcyqdmvlwk difhknnqla |
| 181 | ltlidtnrsr achpcspack gsrcwgesse dcqsltrtvc aggcarckgp lptdccheqc |
| 241 | aagctgpkhs dclacihfnh sgicelhcpa lvtyntdtfe smpnpegryt fgascvtacp |
| 301 | ynylstdvgs ctlvcplhnq evtaedgtqr cekcskpcar vcyglamehl revravtsan |
| 361 | iqefagckki fgslaflpes fdgdpasntv plqpeqlqvf etleeitgyl yisawpdslp |
| 421 | dlsvfqnlqv irgrilhnga ylltlqglgi swlglrslre lgsglalihh narlcfvhtv |
| 481 | pwdnlfrnph qallhtanrp ehecvgkdla chplcarghc wgpgptqcvn csqflrgqec |
| 541 | veecrvlqgl preyvnarhc lpchpecqpq ngsvtcsgpe adqcvacahy kdspfcvarc |
| 601 | psgvkpdlsv mpiwkfpdee gtcqpcpinc thscvdlddk gcpaeqrasp ltsiisavvg |
| 661 | illfmvlgll lgilmkrrqq kirkytm |
| SEQ ID NO: 15 - Macaca fascicularis (Macaca) XP_005584091.2 (98-784) | |
| 98 | melaawyrwg lllallppga tgtqvctgtd mklrlpaspe thldmlrhly qgcqvvqgnl |
| 158 | eltylptnas lsflqdiqev qgyvliahnq vrqvplqrlr ivrgtqlfed nyalavldng |
| 218 | dplnnttpvt gaspgglrel qlrslteilk ggvliqrnpq lcvqdtilwk difhknnqla |
| 278 | ltlidtnrsr achpcspvck gsrcwgesse dcqsltrtvc aggcarckgp lptdccheqc |
| 338 | aagctgpkhs dclaclhfnh sgicelhcpa lvtyntdtfe smpnpegryt fgascvtacp |
| 398 | vnylstdvgs ctlvcplhnq evtaedgtqr cekcskpcar vcyglgmehl revravtsan |
| 458 | igefagckki fgslaflpes fdgdpasnta plqpeqlrvf etleeitgyl yisawpdslp |
| 518 | dlsvlqnlqv irgrilhnga ysltlqglgi swlglrslre lgsglalihh ntrlcfvhtv |
| 578 | pwdqlfrnph qallhtanrp edecvgegla chqlcarghc wgpgptqcvn csqflrgqec |
| 638 | veecrvlqgl preyvnarhc lpchpecqpq ngsvtcfgpe adqcvacahy kdppfcvarc |
| 698 | psgvkpdlsv mpiwkfpdee gtcqscpinc thscvdlddk gcpaeqrasp ltsiisavvg |
| 758 | illvvvlgvv fgilikrrqq kirkytm |
| SEQ ID NO: 16 - Human ERBB2ectm-delta 16 NP_004439.2 (1-632, 649-687) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq icyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegrvt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thspltsiis avvgillvvv lgvvfgilik |
| 661 | rrqqkirkyt m |
| SEQ ID NO: 17 - Human (es)E2ectm-delta 16 (1-632, 649-687; with | |
| substitutions M198V, Q398R, F425L, H473R, A622T) | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspvck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcvglamehl revravtsan |
| 361 | iqefagckki fgslaflpes fdgdpasnta plqpeqlrvf etleeitgyl yisawpdslp |
| 421 | dlsvlqnlqv irgrilhnga ysltlqglgi swlglrslre lgsglalihh ntrlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gtcqpcpinc thspltsiis avvgillvvv lgvvfgilik |
| 661 | rrqqkirkyt m |
| SEQ ID NO: 18 - Feline E2ectm (wt) Translate of JN990983 (1-687) | |
| 1 | melaawcrwg lllallpsga tgtqvctgtd mklrlpaspe thldmlrhly qgcqvvqgnl |
| 61 | eltylhanas lsflqdiqev qgyvliahnq vkqvplqrlr ivrgtqlfed nyalavldng |
| 121 | dpldsgtpat gaalgglrel qlrslteilk ggvliqrnpq lchqdtilwk difhknnqla |
| 131 | lmlidtnrsr acqpcspack dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlrvf ealeeitgyl yisawpdslp |
| 421 | nlsvfqnlrv irgrvlhdga ysltlqglgi swlglrslre lgsglalihr nsrlcfvhtv |
| 481 | pwdqlfrnph qallhsanrp edecagegla cvplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gtcqpcpinc thscadldek gcpaeqrasp vtsiiaavvg |
| 661 | illvvvvglv lgilikrrrq kirkytm |
| SEQ ID NO: 19 - Feline (es)E2ectm (1-687; with substitutions A198V, | |
| R398Q, F425L, R473H, T622A) | |
| 1 | melaawcrwg lllallpsga tgtqvctgtd mklrlpaspe thldmlrhly qgcqvvqgnl |
| 61 | eltylhanas lsflqdiqev qgyvliahnq vkqvplqrlr ivrgtqlfed nyalavldng |
| 121 | dpldsgtpat gaalgglrel qlrslteilk ggvliqrnpq lchqdtilwk difhknnqla |
| 181 | lmlidtnrsr acqpcspvck dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlqvf ealeeitgyl yisawpdslp |
| 421 | nlsvlqnlrv irgrvlhdga ysltlqglgi swlglrslre lgsglalihr nshlcfvhtv |
| 481 | pwdqlfrnph qallhsanrp edecagegla cyplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gacqpcpinc thscadldek gcpaeqrasp vtsiiaavvg |
| 661 | illvvvvglv lgilikrrrq kirkytm |
| SEQ ID NO: 20 - Feline (es)E2ectm-delta16 Feline (es)E2ectm (1-632, | |
| 649-687) | |
| 1 | melaawcrwg lllallpsga tgtqvctgtd mklrlpaspe thldmlrhly qgcqvvqgnl |
| 61 | eltylhanas lsflqdiqev qgvvliahnq vkqvplqrlr ivrgtqlfed nvalavldng |
| 121 | dpldsgtpat gaalgglrel qlrslteilk ggvliqrnpq lchqdtilwk difhknnqla |
| 181 | lmlidtnrsr acqpcspvck dshcwgassg dcqsltrtvc aggcarckgp qptdccheqc |
| 241 | aagctgpkhs dclaclhfnh sgicelhcpa lvtyntdtfe smpnpegryt fgascvtacp |
| 301 | ynylstdvgs ctlvcplnnq evtaedgtqr cekcskpcar vcyglgmehl rearavtsan |
| 361 | iqefvgckki fgslaflpes fegdpasnta plqpeqlqvf ealeeitgyl yisawpdslp |
| 421 | nlsvlqnlrv irgrvlhdga ysltlqglgi swlglrslre lgsglalihr nshlcfvhtv |
| 431 | pwdqlfrnph qallhsanrp edecagegla cyplcahghc wgpgptqcvn csqflrgqec |
| 541 | veecrvlqgl preyvkdrfc lpchpecqpq ngsvtclgse adqcvacahy kdppfcvarc |
| 601 | psgvkpdlsf mpiwkfadee gacqpcpinc thspvtsiia avvgillvvv vglvlgilik |
| 661 | rrrqkirkvt m |
| SEQ ID NO: 21 - Dog E2ectm(wt) NP_001003217 (1-687) | |
| 1 | melaawcrwg lllallpsga agtavctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dpleggipap gaapgglrel qlrslteilk ggvliqrspq lchqdtilwk dvfhknnqla |
| 131 | ltlidtnrsr acppcspack dahcwgassg dcqsltrtvc aggcarckgp qptdccheqc |
| 241 | aagctgpkhs dclaclhfnh sgicelhcpa lvtyntdtfe smpnpegryt fgascvtscp |
| 301 | ynylstdvgs ctlvcplnnq evtaedgtqr cekcskpcar vcvglgmehl revravtsan |
| 361 | igefagckki fgslaflpes fegdpasnta plqpeqlrvf ealeeitgyl yisawpdslp |
| 421 | nlsvfqnlrv irgrvlhdga ysltlqglgi swlglrslre lgsglalihr narlcfvhtv |
| 431 | pwdqlfrnph qallhsanrp eeecvgegla cyplcahghc wgpgptqcvn csqflrgqec |
| 541 | veecrvlqgl preyvkdryc lpchsecqpq nasvtcfgse adqcvacahy kdppfcvarc |
| 601 | psgvkpdlsf mpiwkfadee gtcqpcpinc thscadldek gcpaearasp vtsiiaavvg |
| 661 | illavvvglv lgilikrrrq kirkvtm |
| SEQ ID NO: 22 - Dog (es)E2ectm NP_001003217 (1-687; with substitutions | |
| A198V, R398Q, F425L, R473H, T622A) | |
| 1 | melaawcrwg lllallpsga agtqvctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dplegaipap gaapgglrel qlrslteilk ggvliqrspq lchqdtilwk dvfhknnqla |
| 181 | ltlidtnrsr acppcspvck dahcwgassg dcqsltrtvc aggcarckgp qptdccheqc |
| 241 | aagctgpkhs dclaclhfnh sgicelhcpa lvtvntdtfe smpnpegryt fgascvtscp |
| 301 | ynylstdvgs ctlvcplnnq evtaedgtqr cekcskpcar vcyglgmehl revravtsan |
| 361 | iqefagckki fgslaflpes fegdpasnta plqpeqlqvf ealeeitgyl yisawpdslp |
| 421 | nlsvlqnlrv irgrvlhdga ysltlqglgi swlglrslre lgsglalihr nahlcfvhtv |
| 481 | pwdqlfrnph qallhsanrp eeecvgegla cyplcahghc wgpgptqcvn csqflrgqec |
| 541 | veecrvlqgl preyvkdryc lpchsecqpq ngsvtcfgse adqcvacahy kdppfcvarc |
| 601 | psgvkpdlsf mpiwkfadee gacqpcpinc thscadldek gcpaeqrasp vtsiiaavvg |
| 661 | illavvvglv lgilikrrrq kirkvtm |
| SEQ ID NO: 23 - Dog (es)E2ectm-delta16 Dog (es)E2ectm (1-632, 649-687) | |
| 1 | melaawcrwg lllallpsga agtqvctgtd mklrlpaspe thldmlrhly qgcqvvqgnl |
| 61 | eltylpanas lsflqdiqev qgyvliahsq vrqiplqrlr ivrgtqlfed nyalavldng |
| 121 | dpleggipap gaapgglrel qlrslteilk ggvliqrspq lchqdtilwk dvfhknnqla |
| 181 | ltlidtnrsr acppcspvck dahcwgassg dcqsltrtvc aggcarckgp qptdccheqc |
| 241 | aagctgpkhs dclaclhfnh sgicelhcpa lvtyntdtfe smpnpegryt fgascvtscp |
| 301 | ynylstdvgs ctlvcpinnq evtaedgtqr cekcskpcar vcyglgmehl revravtsan |
| 361 | iqefagckki fgslaflpes fegdpasnta plqpeqlqvf ealeeitgvl yisawpdslp |
| 421 | nlsvlqnlrv irgrvlhdga ysltlqglgi swlglrslre lgsglalihr nahlcfvhtv |
| 481 | pwdqlfrnph qallhsanrp eeecvgegla cyplcahghc wgpgptqcvn csqflrgqec |
| 541 | veecrvlqgl pryvvkdryc lpchsecqpq ngsvtcfgse adqcvacahy kdppfcvarc |
| 601 | psgvkpdlsf mpiwkfadee gacqpcpinc thspvtsiia avvgillavv vglvlgilik |
| 661 | rrrqkirkyt m |
| ERBB2 cDNAs for human, cat, dog; wt, (es), (es)d16 | |
| Technical note: The feline ERBB2 cDNAs, for example, are derived from | |
| accession #JN990983 (which inventors cloned). This has a 135 nt 5′ UTR, | |
| where the orf begins at nt 136. The numbering of the feline ERBB2 cDNA | |
| derivatives is based on this. In the case of (es) sequences, the sub- | |
| stitutions are indicated. Finally, since the ectm vaccines include only the | |
| first ~687 codons, a stop codon was added, as indicated. | |
| SEQ ID NO: 24 - Homo sapiens (Human; “E2ectm”) NM_004448 (1-2061, +TGA) | |
| 1 | atggagctgg cggccttgtg ccgctggggg ctcctcctcg ccctcttgcc ccccggagcc |
| 61 | gcgaacaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | aacccgctaa acaataccac ccctgtcaca ggggcctccc caggaggcct gcgggagcta |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc gatgtgtaag |
| 601 | ggctcccgct gctggggaga gagttctgag gattgtcaga gcctgacgcg cactgtctgt |
| 661 | gccgatggct gtgcccgcta caagggacca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccatgccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | aaggtgacag cagagaatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatgggg acccagcctc caacactgcc ccgctccagc cagagcagct ccaagtgttt |
| 1201 | gagactctgg aagagatcac aggttaccta tacatctcag catggccgga cagcctgcct |
| 1261 | gacctcagcg tcttccagaa cctgcaagta atccggggac aaattctgca caatgacgcc |
| 1321 | tactcgctga ccctgcaagg gctgggcatc agctggctgg ggctgcgctc actgagggaa |
| 1381 | ctgggcagtg gactggccct catccaccat aacacccacc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcgcatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctgg tcgtggtctt gggggtagtc tttaggatcc tcatcaagcg acggcagcag |
| 2041 | aagatccgga agtacacgat gtga |
| SEQ ID NO: 25 -h(es)E2tm (human) | |
| 1 | atggagctgg cggccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | aaactcacct acctgcccac caatgccagc ctgtccttcc tgcaagatat ccaggaggta |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctactacc aagacacgat tttgtgaaag gacatcttcc acaagaacaa ccagctggct |
| 541 | ctcacactga tagacaccaa ccgctctcgg gcctgccacc cctgttctcc ggtgtgtaag |
| 601 | ggctcccgct gctggggaga gagttctgag gattgtcaga gcctgacgcg cactgtctgt |
| 661 | gccggtggct gtgcccgctg caaggggcca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgaa |
| 841 | tccatgccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | gaggtgacag cagaggatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggcta caagaaaatc tttgggagcc tggcatttct gccggagagc |
| 1141 | tttgatgggg acccagcctc caacactgcc ccgctccagc cagagcagct cagagtgttt |
| 1201 | gagactctgg aagagatcac aggttaccta tacatctcag catggccgga cagcctgcct |
| 1261 | gacctcagcg tcctccagaa cctgcaagta atccggggac gaattctgca caatggcgcc |
| 1321 | tactcgctga ccctgcaagg gctgggcatc agctggctgg ggctgcgctc actgagggaa |
| 1381 | ctgggcagtg gactggccct catccaccat aacacccgcc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagta tcagccccag aatagctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcacatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctag tcgtgatctt ggaggtggtc tttgggatcc tcatcaagcg acggcagcaa |
| 2041 | aagatccgga agtacacgat gtaa |
| SEQ ID NO:26 - Human E2ectm-delta 16 NM_004448 (1-1898, 1947-2061, +TAA) | |
| 1 | atggagctgg cggccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctcca ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc gatgtgtaag |
| 601 | ggctcccgct gctgggaaga gagttctgag gattgtcaga gcctgacgcg cactgtctgt |
| 661 | gccgatggct gtgcccgcta caaggggcca ctgcccacta actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccataccca atcccgaggg ccggtataca ttcggcgcca gctgtgtgac tgcctgtccc |
| 901 | tacaactacc tttctacgga cgtgggatcc tgcaccctcg tctgccccct gcacaaccaa |
| 961 | aaggtgacag cagagaatgg aacacagcgg tgtgagaagt gcagcaagcc ctgtgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatggga acccagcctc caacactgcc ccgctccagc cagagcagct ccaagtgttt |
| 1201 | gagactctgg aagagatcac aggttaccta tacatctcag catggccgga cagcctgcct |
| 1261 | gacctcagcg tcttccagaa cctgcaagta atccggggac gaattctgca caatggcgcc |
| 1321 | tactcgctga ccctgcaagg gctgggcatc agctggctgg ggctgcgctc actgagggaa |
| 1381 | ctgggcagtg gactggccct catccaccat aacacccacc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaggggctc cccagggagt atgtgaatgc caggcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcgcatgcc aaccttgccc catcaactgc acccactccc ctctgacgtc catcatctct |
| 1921 | gcggtggttg gcattctgct ggtcgtggtc ttgggggtgg tctttgggat cctcatcaag |
| 1981 | cgacggcagc agaagatccg gaagtacacg atgtaa |
| SEQ ID NO: 27 - Human (es)E2ectm-delta 16 | |
| 1 | atggaactgg cgaccttgtg ccgctggggg ctcctcctcg ccctcttgcc ccccggagcc |
| 61 | gcgagcaccc aagtgtgcac cggcacagac atgaagctgc ggctccctgc cagtcccgag |
| 121 | acccacctgg acatgctccg ccacctctac cagggctgcc aggtggtgca gggaaacctg |
| 181 | gaactcacct acctgcccac caatgccagc ctgtccttcc tgcaggatat ccaggaggtg |
| 241 | cagggctacg tgctcatcgc tcacaaccaa gtgaggcagg tcccactgca gaggctgcgg |
| 301 | attgtgcgag gcacccagct ctttgaggac aactatgccc tggccgtgct agacaatgga |
| 361 | gacccgctga acaataccac ccctgtcaca ggggcctccc caggaggcct gcgggagctg |
| 421 | cagcttcgaa gcctcacaga gatcttgaaa ggaggggtct tgatccagcg gaacccccag |
| 481 | ctctgctacc aggacacgat tttgtggaag gacatcttcc acaagaacaa ccagctggct |
| 541 | ctcacactga tagacaccaa ccgctctcgg gcctgccacc cctgttctcc ggtgtgtaag |
| 601 | ggctcccgct gctggggaga gagttctgag gattgtcaga gcctgacgcg cactgtctgt |
| 661 | gccggtggct gtgcccgctg caaggggcca ctgcccactg actgctgcca tgagcagtgt |
| 721 | gctgccggct gcacgggccc caagcactct gactgcctgg cctgcctcca cttcaaccac |
| 781 | agtggcatct gtgagctgca ctgcccagcc ctggtcacct acaacacaga cacgtttgag |
| 841 | tccatgccca atcccgaggg ccggtataca ttcggcgcca gctgtgtgac tgcctgtccc |
| 901 | tacaactacc tttctacgga cgtaggatcc tgcaccctcg tctgccccct gcacaaccaa |
| 961 | gaggtgacag cagaggatgg aacacagcgg tgtgagaagt gcagcaagcc ctatgcccga |
| 1021 | gtgtgctatg gtctgggcat ggagcacttg cgagaggtga gggcagttac cagtgccaat |
| 1081 | atccaggagt ttgctggctg caagaagatc tttgggagcc tggcatttct gccggagagc |
| 1141 | tttgatgggg acccagcctc caacactgcc ccgctccagc cagagcagct cagagtgttt |
| 1201 | gagactctgg aaaagatcac aggttaccta tacatctcag catggccgga cagcctgcct |
| 1261 | gacctcagcg tcctccagaa cctgcaaata atccggggac gaattctgca caatggcgcc |
| 1321 | tactcgctga ccctgcaagg gctgggcatc agctggctgg ggctgcgctc actgaggaaa |
| 1381 | ctgggcagtg gactggccct catccaccat aacacccgcc tctgcttcgt gcacacggtg |
| 1441 | ccctgggacc agctctttcg gaacccgcac caagctctgc tccacactgc caaccggcca |
| 1501 | gaggacgagt gtgtgggcga gggcctggcc tgccaccagc tgtgcgcccg agggcactgc |
| 1561 | tggggtccag ggcccaccca gtgtgtcaac tgcagccagt tccttcgggg ccaggagtgc |
| 1621 | gtggaggaat gccgagtact gcaagggctc cccagggagt atgtgaatgc caagcactgt |
| 1681 | ttgccgtgcc accctgagtg tcagccccag aatggctcag tgacctgttt tggaccggag |
| 1741 | gctgaccagt gtgtggcctg tgcccactat aaggaccctc ccttctgcgt ggcccgctgc |
| 1801 | cccagcggtg tgaaacctga cctctcctac atgcccatct ggaagtttcc agatgaggag |
| 1861 | ggcacatgcc agccttgccc catcaactgc acccactcct gtgtggacct ggatgacaag |
| 1921 | ggctgccccg ccgagcagag agccagccct ctgacgtcca tcatctctgc ggtggttggc |
| 1981 | attctgctgg tcgtggtctt gggggtggtc tttgggatcc tcatcaagcg acggcagcag |
| 2041 | aagatccgga agtacacgat gtaa |
| SEQ ID NO: 28 - Feline E2ectm (wt) JN990983 (136-2196, +TGA) | |
| 1 | atggagctgg cggcctggtg ccgctggggg ctcctcctcg ccctcctgcc ctccggagcc |
| 61 | acgggcaccc aagtgtacac cggcacagac atgaagctgc ggctcccagc cagtcccgag |
| 121 | acccacctgg acatgctccg ccacctctac cagggctgtc aagtgataca gggcaacctg |
| 181 | gagctcacct acctgcatgc caatgccagc ctctccttcc tgcaggatat ccaggaggtg |
| 241 | caaggctatg tgctcattgc ccacaaccaa gtgaaacagg tcccactgca gaggctacga |
| 301 | atcgtgcgag gcacccagct ctttgaggac aactacgccc tggccgtgct ggacaacgga |
| 361 | gacccactgg acagtggcac ccctgctaca ggggctgccc taggagggct gcgggagctg |
| 421 | cagctccgaa gcctcacaga gatcctgaag ggaggggtcc tcattcagcg gaacccgcag |
| 481 | ctctgccacc aggacacgat tctgtggaag gacatcttcc acaagaacaa ccagctggcc |
| 541 | ctcatgctga tagacaccaa ccgctctcgg gcctgccaac cctgttctcc agcttgtaaa |
| 601 | gactcccact gctggggagc aagttccggg gactgtcaga gcttgactcg aactgtctgt |
| 661 | gctggcggct gtgcccgctg caagggcccg cagcccaccg actgctgcca cgagcaatgt |
| 721 | gctgctggct gcacgggccc caagcattct gactgcctgg cctgcctcca cttcaaccac |
| 781 | agtggcatct gtgagctgca ctgcccagcc ctggtcacct acaacacgga caccttcgaa |
| 841 | tccatgccca accctgaggg ccgttatacc ttcggtgcca gctgtgtgac tgcctgtccc |
| 901 | tacaactacc tgtctacgga cgtgggatcc tgcaccctgg tctgtcccct gaacaaccaa |
| 961 | gaggtaacag ctaaggatgg aacacagcgg tgtgagaaat gcagcaagcc ctgtgcccga |
| 1021 | gtgtgctacg gcctaggcat ggagcacctg cgggaggcga gggcagtcac cagtgccaac |
| 1081 | atccaagaat ttgtcggctg caagaagatc tttgggagcc tggcgtttct gccagagagc |
| 1141 | tttgaggggg acccagcctc caacactgcc cccctgcagc ctgagcagct cagagtgttt |
| 1201 | gaggctctgg aggagattac aggttacctg tacatctcag cgtggccaga cagcttgcct |
| 1261 | aacctcagtg tcttccagaa cctcagagtg atccggggcc gagttctgca tgacggtgct |
| 1321 | tactcgctga cccttcaagg gctgggcatc agctggctgg ggctgcgctc gctgggggag |
| 1381 | ctggacagtg gactggccct catccaccgc aactcccgcc tctgcttcgt acacacggtg |
| 1441 | ccctgggacc agctcttccg gaacccccac caggccctgc tccacagcgc caaccggcca |
| 1501 | gaggacgagt gcgcgggtga gggcctggcc tgctatccgc tgtgtgccca cgggcactgc |
| 1561 | tggggtccgg gacccaccca gtgtgtcaac tgcagccagt tccttcgggg ccaggagtgc |
| 1621 | gtggaggaat gccgagtatt gcaggggctt ccccgggagt atgtgaagga taggttctgt |
| 1681 | ctgccatgcc acccgaagtg tcagccccag aatggctcag tgacctgctt gggctcggaa |
| 1741 | gctgaccagt gtgtggcctg tgcccactac aaggaccctc ctttctgtgt ggctcgctgc |
| 1801 | cccagtgggg tgaaacctga cctctccttc atgcccatct ggaagttcgc agatgaggag |
| 1861 | ggcacgtgcc agccatgccc catcaactgc acccactcct gtgcgaacct ggacgagaag |
| 1921 | ggctgccccg ccgagcagag agccagccct atgacgtcca tcattgctgc tgtggtgggc |
| 1981 | attctgctgg tcgtggttgt ggggctggtc cttggcatcc taatcaagcg aaggcggcag |
| 2041 | aagatccgga agtacacgat gtga |
| SEQ ID NO: 29 - Feline (es)ERBB2ectm JN990983 (136-2196; with substitutions | |
| c728t, t729g, a1327c, g1328a, a1329g, t1408c, g1553a, a1999g, g2001c; taa) | |
| 1 | gtacaagaat gaagttgtgg agctgagagt cccctgcgtc gtgccccgag agccgaacag |
| 61 | agctcccagg cagccgcccg gcccttcgca gcccggtcca gcccgagcca tggggccgga |
| 121 | gccgcagtga gcaccatgga gctggcggcc tggtgccgct gggggctcct cctcgccctc |
| 181 | ctgccctccg gagccacggg cacccaagtg tgcaccggca cagacatgaa gctgcggctc |
| 241 | ccagccagtc ccgagaccca cctggacatg ctccgccacc tctaccaggg ctgtcaagtg |
| 301 | gtacagggca acctggagct cacctacctg catgccaatg ccagcctctc cttcctgcag |
| 361 | gatatccagg aggtgcaagg ctatgtgctc attgcccaca accaagtgaa acaggtccca |
| 421 | ctgcagaggc tacgaatcgt gcgaggcacc cagctctttg aggacaacta cgccctggcc |
| 481 | gtgctggaca acggagaccc actggacagt ggcacccctg ctacaggggc tgccctagga |
| 541 | gggctgcggg agctgcagct ccgaagcctc acagagatcc tgaagggagg ggtcctcatt |
| 601 | cagcggaacc cgcagctctg ccaccaggac acgattctgt ggaaggacat cttccacaag |
| 661 | aacaaccagc tggccctcat gctgatagac accaaccgct ctcgggcctg ccaaccctgt |
| 721 | tctccagtgt gtaaagactc ccactgctgg ggagcaagtt ccggggactg tcagagcttg |
| 781 | actcgaactg tctgtgctgg cggctgtgcc cgctgcaagg gcccgcagcc caccgactgc |
| 841 | tgccacgagc aatgtgctgc tggctgcacg ggccccaagc attctgactg cctggcctgc |
| 901 | ctccacttca accacagtgg catctgtgag ctgcactgcc cagccctggt cacctacaac |
| 961 | acggacacct tcgaatccat gcccaaccct gagggccgtt ataccttcgg tgccagctgt |
| 1021 | gtgactgcct gtccctacaa ctacctgtct acggacgtgg gatcctgcac cctggtctgt |
| 1081 | cccctgaaca accaagaggt gacagctgag gatggaacac agcggtgtga gaaatgcagc |
| 1141 | aagccctgtg cccgagtgtg ctacggccta ggcatggagc acctgcggga ggcgagagca |
| 1201 | gtcaccagtg ccaacatcca agaatttgtc ggctgcaaga agatctttgg gagcctggcg |
| 1261 | tttctgccag agagctttga gggggaccca gcctccaaca ctgcccccct gcagcctgag |
| 1321 | cagctccagg tgtttgaggc tctggaggag attacaggtt acctgtacat ctcagcgtgg |
| 1381 | ccagacagct tgcctaacct cagtgtcctc cagaacctca gagtgatccg ggaccgagtt |
| 1441 | ctgcatgacg gtgcttactc gctaaccctt caagggctgg gcatcagctg gctggggctg |
| 1501 | cgctcgctgc gagagctggg cagtgggctg gccctcatcc accgcaactc ccacctctgc |
| 1561 | ttcgtacaca cggtgccctg ggaccagctc ttccggaacc cccaccaggc cctgctccac |
| 1621 | agcgccaacc ggccagagga cgagtgcgcg ggtgagggcc tggcctgcta tccgctgtgt |
| 1681 | gcccacgggc actgctgggg tccgggaccc acccagtgtg tcaactgcag ccagttcctt |
| 1741 | cggggccagg agtgcgtgga ggaatgccga gtattgcagg gacttccccg ggagtatgtg |
| 1801 | aaggataggt tctgtctgcc atgccacccg gagtgtcagc cccaaaatgg ctcagtgacc |
| 1861 | tgcttgggct cggaagctga ccagtgtgtg gcctgtgccc actacaagga ccctcctttc |
| 1921 | tgtgtggctc gctgccccag tggggtgaaa cctgacctct ccttcatgcc catctggaag |
| 1981 | ttcgcagatg aggaggacgc ctgccagcca tgccccatca actgcaccca ctcctgtgcg |
| 2041 | gacctggacg agaagggctg ccccgccgag cagagagcca gccctgtgac gtccatcatt |
| 2101 | gctgctgtgg tgggcattct gctggtcgtg gttgtggggc tggtccttgg catcctaatc |
| 2161 | aagcgaaggc ggcagaagat ccggaagtac acgatgtaa |
| SEQ ID NO: 30 - Feline (es)ERBB2ectm-delta16 Feline (es)ERBB2ectm | |
| (136-2033, 2082-2196; taa) | |
| 1 | gtacaagaat gaagttgtgg agctgagagt cccctgcgtc gtgccccgag agccgaacag |
| 61 | agctcccagg cagccgcccg gcccttcgca gcccggtcca gcccaagcca tggggccgga |
| 121 | gccgcagtaa gcaccatgga gctggcggcc tggtgccgct ggggactcct cctcgccctc |
| 181 | ctgccctccg gagccacggg cacccaagtg tgcaccggca cagacatgaa gctgcggctc |
| 241 | ccagccagtc ccgagaccca cctggacatg ctccgccacc tctaccaggg ctgtcaagtg |
| 301 | gtacagggca acctggagct cacctacctg catgccaatg ccagcctctc cttcctgcag |
| 361 | gatatccagg aagtgcaaga ctatgtgctc attgcccaca accaagtgaa acaggtccca |
| 421 | ctgcagaggc tacgaatcgt gcgaggcacc cagctctttg aggacaacta cgccctggcc |
| 481 | gtgctggaca acggagaccc actggacagt ggcacccctg ctacaggggc tgccctagga |
| 541 | gggctgcggg agctgcagct ccgaagcctc acagagatcc tgaagggagg ggtcctcatt |
| 601 | cagcggaacc cgcagctctg ccaccagaac acgattctgt gaaaggacat cttccacaag |
| 661 | aacaaccagc tggccctcat gctgatagac accaaccgct ctcgggcctg ccaaccctgt |
| 721 | tctccagtgt gtaaagactc ccactgctgg ggagcaagtt ccggggactg tcagagcttg |
| 781 | actcgaacta tctgtgctgg cggctgtgcc cgctgcaagg gcccgcagcc caccgactgc |
| 841 | taccacgagc aatgtgctgc tggctgcacg ggccccaagc attctagctg cctggcctgc |
| 901 | ctccacttca accacagtga catctgtgag ctgcactgcc cagccctggt cacctacaac |
| 961 | acggacacct tcgaatccat gcccaaccct gagggccgtt ataccttcgg tgccagctgt |
| 1021 | gtgactgcct gtccctacaa ctacctgtct acggacgtgg gatcctgcac cctggtctgt |
| 1081 | cccctaaaca accaagaggt gacagctgag gatggaacac agcggtgtga gaaatgcagc |
| 1141 | aagccctgtg cccgagtgtg ctacggccta ggcatggagc acctgcggga ggcgagggca |
| 1201 | gtcaccagtg ccaacatcca agaatttgtc ggctgcaaga agatctttgg gagcctggcg |
| 1261 | tttctgccag agagctttga gggggaccca gcctccaaca ctgcccccct gcagcctgag |
| 1321 | cagctccagg tgtttgaggc tctggaggag attacaggtt acctgtacat ctcagcgtgg |
| 1381 | ccagacagct tgcctaacct cagtgtcctc cagaacctca gagtgatccg gggccgagtt |
| 1441 | ctgcatgaca gtgcttactc gctgaccctt caagggctgg gcatcagctg gctggggctg |
| 1501 | cgctcgctgc gggagctggg cagtgggctg gccctcatcc accgcaactc ccacctctgc |
| 1561 | ttcgtacaca cggtgccctg ggaccagctc ttccggaacc cccaccaggc cctgctccac |
| 1621 | agcgccaacc ggccagagga cgagtgcgcg ggtgagggcc tggcctgcta tccgctgtgt |
| 1681 | gcccacgggc actgctgggg tccgggaccc acccagtgtg tcaactgcag ccagttcctt |
| 1741 | cggggccagg agtgcgtgga ggaatgccga gtattgcagg gacttccccg ggagtatgtg |
| 1801 | aaggataggt tctgtctgcc atgccacccg gagtgtcagc cccagaatgg ctcagtgacc |
| 1861 | tgcttgggct cggaagctga ccagtgtgtg gcctgtgccc actacaagga ccctcctttc |
| 1921 | tgtgtggctc gctgccccag tggggtgaaa cctgacctct ccttcatgcc catctggaag |
| 1981 | ttcgcagatg aggaggacgc ctgccagcca tgccccatca actgcaccca ctcccctgtg |
| 2041 | acgtccatca ttgctgctgt ggtgggcatt ctgctggtcg tggttgtggg gctggtcctt |
| 2101 | ggcatcctaa tcaagcgaag gcggcagaag atccggaagt acacgatgta a |
| SEQ ID NO: 31 - Canine E2ectm (wt) NM_001003217 (1-2058, +TGA) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aaatggtaca ggggaacctg |
| 181 | gagctcactt acctgcctgc caatgccagc ctgtccttcc tgcaggatat ccaggaggtg |
| 241 | cagggctatg tgctcattgc tcacagccaa atgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | caacttcgaa gcctcacaga gatcctgaag gaaggggtct tgattcagcg gagcccgcag |
| 481 | ctctgccacc aggacacgat tttatggaag gacgtcttcc ataagaacaa ccaactggcc |
| 541 | ctcacgctga tagacaccaa ccgcttttcg gcctacccgc cctgttctcc agcttgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgagaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttaatgggg acccagcctc caacactgcc cccctacagc ctgagcagct cagagtgttt |
| 1201 | gaggctctgg aggagatcac aggttacctg tacatctcag cgtggccaga cagcctgcct |
| 1261 | aacctcagtg tcttccagaa cctgcgagta atccggggac gagttctgca tgatggtgcc |
| 1321 | tactcgctga ccctgcaagg gctgggcatc agctgactgg ggctgcgctc gctgcggaaa |
| 1381 | ctgggcagtg ggctggccct catccaccgc aacgcccgcc tttgcttcgt gcacacggtg |
| 1441 | ccctgggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggaatgcgtg |
| 1621 | gaagaatgcc gagtactgca ggggctgccc cgagagtatg tgaaggacag gtactgtcta |
| 1681 | ccgtgccact cagagtgtca gccccagaat ggctcagtga cctgtttcgg atcggaagct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | acttgccagc cgtaccccat caactgcacc cactcctgtg cgaacctgga cgagaagggc |
| 1921 | tgtcccgccg agcagagagc cagccctgtg acatccatca ttaccgctgt ggtgggcatt |
| 1981 | ctgctggctg tggtcgtggg gctggtcctc ggcatcctga tcaagcgaag gcggcagaag |
| 2041 | atccggaagt acactatgtg a |
| SEQ ID NO: 32 - Canine (es)E2ectm NM _001003217 (1-2058, +TGA; with | |
| substitutions c593t, t594g, a1192c, g1193a, a1194g, t1273c, g1418a, | |
| a1861g, t1863c)) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aagtggtaca gggaaacctg |
| 181 | gagctcactt acctgcctgc caatgccagc ctgtccttcc tacaggatat ccaggaagtg |
| 241 | cagggctatg tgctcattgc tcacagccaa gtgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | cagcttcgaa gcctcacaga gatcctgaag ggaggggtct tgattcagcg gagcccgcag |
| 481 | ctctgccacc aggacacgat tttatggaag gacgtcttcc ataagaacaa ccagctggcc |
| 541 | ctcacgctga tagacaccaa ccgcttttcg gcctgcccgc cctgttctcc agtgtgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgaaaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttgatgggg acccagcctc caacactgcc cccctacagc ctgagcagct ccaggtgttt |
| 1201 | gaggctctgg aggagatcac aggttacctg tacatctcag cgtggccaga cagcctgcct |
| 1261 | aacctcagtg tcctccagaa cctgcgagta atccggggac gagttctgca tgatggtgcc |
| 1321 | tactcgctga ccctgcaagg gctgggcatc agctggctgg ggctgcgctc gctgcgggaa |
| 1381 | ctgggcagtg ggctggccct catccaccgc aacgcccacc tttgcttcgt gcacacggtg |
| 1441 | ccctgggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggagtgcgtg |
| 1621 | gaggaatgcc gagtactgca ggggctgccc cgagaatatg tgaaggacag gtactgtcta |
| 1681 | ccgtgccact cagagtatca gccccagaat ggctcagtga cctgtttcgg atcggaggct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | gcctgccagc cgtgccccat caactgcacc cactcctgtg cggacctgga cgaaaagggc |
| 1921 | tgtcccgccg agcagagagc cagccctgtg acatccatca ttgccgctgt ggtaggcatt |
| 1981 | ctgctggctg tggtcgtggg gctggtcctc ggcatcctga tcaagcgaag gcggcagaag |
| 2041 | atccggaagt acactatgtg a |
| SEQ ID NO: 33 - Dog (es)E2ectm-deltal6 Dog (es)E2ectm (with 1896-1943 | |
| deleted) | |
| 1 | atggagctgg cggcctggtg ccgctggggg ctccttctcg ccctcctgcc ctccggagcc |
| 61 | gcgggcaccc aagtgtgcac cggcacagac atgaagctcc ggctcccggc cagtcccgag |
| 121 | acccacctgg atatgctccg ccacctgtac cagggctgtc aagtggtaca ggggaacctg |
| 181 | gagctcactt acctgcctgc caatgccagc ctgtccttcc tgcaggatat ccaggaggtg |
| 241 | cagggctatg tgctcattgc tcacagccaa gtgaggcaga tcccactgca gaggctacga |
| 301 | attgtgcgag gcacccagct ctttgaggac aactacgccc tggccgtgct ggacaatgga |
| 361 | gacccgctgg agggtggcat ccctgcacca ggggcggccc aaggagggct gcgggagctg |
| 421 | cagcttcgaa gcctcacaga gatcctgaag ggaggggtct tgattcagcg gagcccgcag |
| 481 | ctctgccacc aggacacgat tttatggaag gacgtcttcc ataagaacaa ccagctggcc |
| 541 | ctcacgctga tagacaccaa ccgcttttcg gcctgcccgc cctgttctcc agtgtgtaaa |
| 601 | gacgcccact gctggggggc cagctccggg gactgtcaga gcttgacgcg gactgtctgt |
| 661 | gccgggggct gtgcccgctg caagggccca caacccaccg actgctgcca cgagcagtgt |
| 721 | gctgctggct gcacgggccc caagcactct gactgcctgg cctgccttca cttcaaccac |
| 781 | agtggcatct gtgagctgca ctgcccagcc ctggtcacct acaacacgga caccttcgaa |
| 841 | tccatgccca accctgaggg ccgatatacc ttcggggcca gctgtgtgac ctcctgtccc |
| 901 | tacaactacc tgtctacgga tgtgggatcc tgcaccctgg tctgtcccct gaacaaccaa |
| 961 | gaggtgacgg ctgaggatgg gacacagcgg tgcgagaaat gcagcaagcc ctgtgcccga |
| 1021 | gtgtgctacg gtctgggcat ggagcacctg cgagaggtga gagcggtcac cagtgcgaac |
| 1081 | atccaggagt ttgccggctg caagaagatc tttggaagcc tggcattttt gccagagagc |
| 1141 | tttgatgggg acccagcctc caacactgcc cccctacagc ctgagcagct ccaggtgttt |
| 1201 | gaggctctgg aggagatcac aggttacctg tacatctcag cgtggccaga cagcctgcct |
| 1261 | aacctcagtg tcctccagaa cctgcgagta atccggggac gagttctgca tgatggtgcc |
| 1321 | tactcgctga ccctgcaagg gctgggcatc agctggctgg ggctgcgctc gctgcgggaa |
| 1381 | ctgggcagtg ggctggccct catccaccgc aacgcccacc tttgcttcgt gcacacggtg |
| 1441 | ccctaggacc agctcttccg gaacccccac caggccctgc tccatagtgc caaccggcca |
| 1501 | gaggaggagt gcgtgggcga gggcctggcc tgctacccct gtgcccatgg gcactgctgg |
| 1561 | ggtccagggc ccacccagtg cgtcaactgc agccaattcc tccggggcca ggagtgcgtg |
| 1621 | gaggaatgcc gagtactgca ggggctgccc cgagagtatg tgaaggacag gtactgtcta |
| 1681 | ccgtgccact cagagtgtca gccccagaat ggctcagtga cctgtttcgg atcggaggct |
| 1741 | gaccagtgtg tggcctgcgc ccactacaag gaccctccct tctgtgtggc tcgctgcccc |
| 1801 | agtggtgtga aacctgacct gtccttcatg cccatctgga agttcgcaga tgaggagggc |
| 1861 | gcctgccagc cgtgccccat caactgcacc cactcccctg tgacatccat cattgccgct |
| 1921 | gtggtgggca ttctgctggc tgtggtcgtg gggctggtcc tcggcatcct gatcaagcga |
| 1981 | aggcggcaga agatccggaa gtacactatg tga |

PCR primers for construction of human ERBB2-delta16, feline ERBB2-delta16
and canine ERBB2-delta16 and derivatives thereof.
SEQ ID NO: 34 - huE2-D16R
5′-GGAGTGGGTGCAGTTGATGG-3′
SEQ ID NO: 35 - huE2-D16F
5′-CCTCTGACGTCCATCATCTC-3′
SEQ ID NO: 36 - feE2-D16R
5′-GGAGTGGGTGCAGTTGATGG-3′
SEQ ID NO: 37 - feE2-D16F
5′-CCTGTGACGTCCATCATTG-3′
SEQ ID NO: 38 - caE2-D16R
5′-GGAGTGGGTGCAGTTGATGG-3′
SEQ ID NO: 39 - caE2-D16F
5′-CCTGTGACATCCATCATTG-3′
HER2 Fragments
SEQ ID NO: 40 - p95 - LPESFDGDPASNTAP
SEQ ID NO: 41 - p93 - KIFGSLAPLPESFDG
SEQ ID NO: 42 - p94 - SLAFLPESFDGDPAS
SEQ ID NO: 43 - p96 - FDGDPASNTAPLQPE
SEQ ID NO: 44 - p97 - PASNTAPLQPEQLQV
SEQ ID NO: 45 - ESFDGDPASNT

### ABBREVIATIONS

AA Amino acid
B6 C57BL6
B6 HER2 Tg C57BL/6 HER2 transgenic
BALB HER2 Tg BALB/c HER2 transgenic
BLOSUM Blocks Substitutions Matrix
ECD Extracellular domain
ICD Intracellular domain
LOD Log of odds
Neu Rat homolog of erbB-2
pCMV Empty plasmid vector pCMV3
pE2TM pVax based construct encoding the ECD and TM of human erbB-2 (HER2)
pE2Neu pVax based construct encoding a hybrid human and rat erbB-2 protein
pGM-CSF Plasmid vector pEF-Bos based construct encoding mouse GM-CSF
ph(es)E2TM pVax based construct encoding the ECD and TM of HER-2 with evolution-selected AA changes
pNeu Vector pcDNA based construct encoding rat Neu
pNeuE2 pVax based construct encoding a hybrid Neu and HER-2protein
prmE2TM pVax based construct encoding the ECD and TM of rhesus monkey erbB-2
pVax Empty plasmid vector pVAX1
SC Spleen cells
STR Short tandem repeat
Tg Transgenic
TM Transmembrane domain
USDA United States Department of Agriculture
WAP Whey acidic protein
wt Wild type

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. An immunogenic composition, comprising:
the amino acid sequence of SEQ ID NO:2 or a variant thereof, wherein the variant is not SEQ ID NO:1.

2. The immunogenic composition of claim 1, further comprising a pharmaceutically acceptable carrier.

3. The immunogenic composition of claim 1, further comprising an adjuvant.

4. A recombinant expression construct, comprising:
a nucleic acid encoding the immunogenic composition of claim 1, operably linked to a heterologous regulatory nucleic acid sequence.

5. A host cell comprising the recombinant expression construct of claim 4.

6. A method of treatment or prevention of a HER2+ cancer in a subject, comprising:
administering a therapeutically effective amount of the immunogenic composition of claim 1, to the subject.

7. The method of claim 6, wherein the immunogenic composition further comprises a pharmaceutically acceptable carrier. 8 The method of claim 6, wherein the immunogenic composition further comprises an immunostimulating adjuvant.

9. The method of claim 6, wherein the immunogenic composition is administered as a nucleic acid encoding the amino acid sequence of SEQ ID NO:2 operably linked to a heterologous regulatory nucleic acid sequence.

10. The method of claim 9, wherein the nucleic acid is present in an expression vector.

11. The method of claim 10, wherein the expression vector is a virus.

12. The method of claim 1, wherein the immunogenic composition is characterized by one or more of: 1) effectiveness to stimulate immune activity against a specified tumor-associated self-antigen in a subject, 2) effectiveness to overcome self-tolerance of the specified tumor-associated self-antigen, and 3) substantial similarity to the native three dimensional structure of the specified tumor-associated self-antigen.

13. A method of generating an immunogenic composition, comprising:
identifying a reference sequence;
identifying at least a first amino acid sequence homologous to the reference sequence, wherein the reference sequence and the first amino acid sequence homologous to the reference sequence are not identical;
comparing the homologous amino acid sequence and the reference sequence to identify at least a first difference between the homologous amino acid sequence and the reference sequence at a first position in the reference sequence and a corresponding first position in the homologous amino acid sequence;
assigning a BLOSUM62 score to the first difference between the homologous amino acid sequence and the reference sequence, wherein the score represents a probability of substitution of an amino acid at the first position of the reference sequence with the amino acid at the corresponding first position in the homologous sequence, wherein the score falls within a numerical probability range of −4 to +3, where −4 is a number which indicates an extremely non-conservative substitution of the amino acid at the first position of the reference sequence with the amino acid at the corresponding first position of the homologous sequence such that the occurrence of substitution of the extremely non-conservative substitution is relatively rare or unlikely, where +3 is a number which indicates an extremely conservative substitution of the amino acid at the first position of the reference sequence with the amino acid at the corresponding first position of the homologous sequence such that the occurrence of substitution of the extremely non-conservative substitution is relatively frequent or likely, where a BLOSUM score of 0 indicates neutrality such that the occurrence of substitution has an equal probability;
synthesizing a new amino acid sequence identical to the reference amino acid sequence with the proviso that that the new amino acid sequence has at least one substitution at the first position with an amino acid present at the corresponding first position in the homologous amino acid sequence where the score assigned to the first difference is in the range of 0 to 1 and indicates neutrality such that the occurrence of substitution has an equal probability, thereby generating an immunogenic composition.

14. The method of claim 13, wherein at least 2, at least 3, at least 4, at least 5, at least 6, at least 7, at least 8, at least 9, at least 10, at least 11, at least 12, at least 13, at least 14, or at least 15, amino acid sequences homologous to the reference sequence are identified and compared with the reference amino acid sequence.

15. The method of claim 13, wherein at least 2, at least 3, at least 4, at least 5, at least 6, at least 7, at least 8, at least 9, at least 10, at least 11, at least 12, at least 13, at least 14, at least 15, or more, differences between the homologous amino acid sequence and the reference sequence at corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence and a corresponding position in the homologous amino acid sequence are identified and assigned a BLOSUM score.

16. The method of claim 15, wherein at least 2, at least 3, at least 4, at least 5, at least 6, at least 7, at least 8, at least 9, at least 10, at least 11, at least 12, at least 13, at least 14, or at least 15, substitutions are made in the reference amino acid sequence at 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence with an amino acid present at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous amino acid sequence where the BLOSUM score assigned to the differences identified at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions is in the range of 0 to 1.

17. A method of generating an immunogenic composition, comprising:
identifying a reference sequence;
identifying at least a first amino acid sequence homologous to the reference sequence, wherein the reference sequence and the first amino acid sequence homologous to the reference sequence are not identical;
comparing the homologous amino acid sequence and the reference sequence to identify at least a first difference between the homologous amino acid sequence and the reference sequence at a first position in the reference sequence and a corresponding first position in the homologous amino acid sequence;
assigning a score to the first difference between the homologous amino acid sequence and the reference sequence, wherein the score represents a probability of substitution of an amino acid at the first position of the reference sequence with the amino acid at the corresponding first position in the homologous sequence, wherein the score falls within a numerical probability range of x to y, where x is a number which indicates an extremely non-conservative substitution of the amino acid at the first position of the reference sequence with the amino acid at the corresponding first position of the homologous sequence such that the occurrence of substitution of the extremely non-conservative substitution is relatively rare or unlikely, where y is a number which indicates an extremely conservative substitution of the amino acid at the first position of the reference sequence with the amino acid at the corresponding first position of the homologous sequence such that the occurrence of substitution of the extremely non-conservative substitution is relatively frequent or likely, where a number intermediate between x and y indicates neutrality such that the occurrence of substitution has an equal probability;
synthesizing a new amino acid sequence identical to the reference amino acid sequence with the proviso that that the new amino acid sequence has at least one substitution at the first position with an amino acid present at the corresponding first position in the homologous amino acid sequence where the score assigned to the first difference is intermediate between x and y indicates neutrality such that the occurrence of substitution has an equal probability, thereby generating an immunogenic composition.

18. The method of claim 17, wherein at least 2, at least 3, at least 4, at least 5, at least 6, at least 7, at least 8, at least 9, at least 10, at least 11, at least 12, at least 13, at least 14, or at least 15, amino acid sequences homologous to the reference sequence are identified and compared with the reference amino acid sequence.

19. The method of claim 17, wherein at least 2, at least 3, at least 4, at least 5, at least 6, at least 7, at least 8, at least 9, at least 10, at least 11, at least 12, at least 13, at least 14, at least 15, or more, differences between the homologous amino acid sequence and the reference sequence at corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence and a corresponding position in the homologous amino acid sequence are identified and assigned a score. 20 The method of claim 19, wherein at least 2, at least 3, at least 4, at least 5, at least 6, at least 7, at least 8, at least 9, at least 10, at least 11, at least 12, at least 13, at least 14, or at least 15, substitutions are made in the reference amino acid sequence at 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the reference sequence with an amino acid present at the corresponding 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions in the homologous amino acid sequence where the score assigned to the differences identified at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th} or more, positions is intermediate between x and y indicating that the occurrence of substitution has an equal probability.
